SEQUENCE LISTING (10 sequences)

SEQ ID NO: 1 - protein, 119 aa, synthetic construct
  source 1..119: note = An artificially synthesized polypeptide sequence
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSS

SEQ ID NO: 2 - protein, 107 aa, synthetic construct
  source 1..107: note = An artificially synthesized polypeptide sequence
DIQMTQSPSSLSASVGDSVTITCQASTDISSHLNWYQQKPGKAPELLIYYGSHLLSGVPSRFSGSGSGTDFTFTISSLEAEDAATYYCGQGNRLPYTFGQGTKVEIE

SEQ ID NO: 3 - protein, 443 aa, synthetic construct
  source 1..443: note = An artificially synthesized polypeptide sequence
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHAHYTQKSLSLSP

SEQ ID NO: 4 - protein, 214 aa, synthetic construct
  source 1..214: note = An artificially synthesized polypeptide sequence
DIQMTQSPSSLSASVGDSVTITCQASTDISSHLNWYQQKPGKAPELLIYYGSHLLSGVPSRFSGSGSGTDFTFTISSLEAEDAATYYCGQGNRLPYTFGQGTKVEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 6 aa, synthetic construct
  source 1..6: note = An artificially synthesized sequence
HDHAWS

SEQ ID NO: 6 - protein, 16 aa, synthetic construct
  source 1..16: note = An artificially synthesized sequence
FISYSGITNYNPSLQG

SEQ ID NO: 7 - protein, 10 aa, synthetic construct
  source 1..10: note = An artificially synthesized sequence
SLARTTAMDY

SEQ ID NO: 8 - protein, 11 aa, synthetic construct
  source 1..11: note = An artificially synthesized sequence
QASTDISSHLN

SEQ ID NO: 9 - protein, 7 aa, synthetic construct
  source 1..7: note = An artificially synthesized sequence
YGSHLLS

SEQ ID NO: 10 - protein, 9 aa, synthetic construct
  source 1..9: note = An artificially synthesized sequence
GQGNRLPYT

(19) United States
(12) Patent Application Publication
(10) Pub. No.: US 2026/0297203 A1
(43) Pub. Date: Oct. 1, 2026

(54) TREATMENT OF THYROID EYE DISEASE WITH SATRALIZUMAB

(71) Applicants: Chugai Seiyaku Kabushiki Kaisha, Tokyo (JP); Genentech, Inc., South San Francisco, CA (US); Hoffmann-La Roche, Inc., Little Falls, NJ (US)

(72) Inventors: Takatoshi Ozawa, Tokyo (JP); Hiroaki Ida, Tokyo (JP); Shunsuke Yoshida, Tokyo (JP); Oluwatobi Idowu, South San Francisco, CA (US); Sian Lennon-Chrimes, Welwyn Garden City (GB); Zdenka Haskova, South San Francisco, CA (US); Christopher Brittain, South San Francisco, CA (US)

(73) Assignees: Chugai Seiyaku Kabushiki Kaisha, Tokyo (JP); Genentech, Inc., South San Francisco, CA (US); Hoffmann-La Roche, Inc., Little Falls, NJ (US)

(21) Appl. No.: 19/477,393

(22) PCT Filed: Apr. 26, 2024
(86) PCT No.: PCT/JP2024/016379

§ 371 Date: Oct. 21, 2025

Related U.S. Application Data
(60) Provisional application No. 63/498,890, filed on Apr. 28, 2023.

(51) Int. Cl. C07K 16/28; A61P 5/14

(52) U.S. Cl. CPC C07K 16/2866; A61P 5/14; C07K 2317/24

(57) ABSTRACT

All currently available treatment options for thyroid eye disease (TED) carry substantial potential safety risks. The present disclosure provides a means for treating or preventing TED comprising an IL-6 inhibitor such as anti-IL-6 receptor antibody or antigen binding fragment thereof such as satralizumab.

### TECHNICAL FIELD

The present disclosure relates to a method of treating or preventing thyroid eye disease (abbreviated as TED) by administering an anti-IL-6 receptor antibody or antigen binding fragment thereof to a subject in need thereof. The present disclosure also relates to a medicament or a pharmaceutical composition for treating or preventing TED comprising an anti-IL-6 receptor antibody or antigen binding fragment thereof.

### BACKGROUND ART

TED, also referred to as Graves ophthalmopathy or orbitopathy (GO) or thyroid-associated orbitopathy, is a chronic inflammatory disorder predominantly associated with Graves disease (Bartley 1994 [NPL 1]). While 90% of TED is associated with Graves disease, 5% occurs in hypothyroid state and 5% in euthyroid individuals. The clinical presentation of TED is highly variable in severity, ranging from mild disease which may be self-limiting, to severe sight-threatening disease in 3%-7% of cases (Wiersinga and Bartalena 2002 [NPL 2]; Barrio-Barrio et al. 2015 [NPL 3]). Moderate-to-severe disease can be associated with permanent visual impairment, as well as morphologic disfiguring changes to the face, related to proptosis, lid lag, and gaze disturbances, all of which impact patient quality of life (Gerding et al. 1997 [NPL 4]; Kahaly et al. 2002 [NPL 5]; Wiersinga 2012 [NPL 6]).

TED is an autoimmune disease and although the underlying pathophysiological mechanism is not completely understood, it is presumed to be mediated through the activation of orbital fibroblasts by autoantibodies directed against thyroid antigens, such as thyrotropin receptor (TSH receptor; TSHR) autoantibodies and insulin-like growth factor-1 receptor autoantibodies (Ezra et al. 2012 [NPL 7]). Typically, TED has a progressive (active) inflammatory phase, which eventually leads to the emergence of fibrosis and a mixed inflammatory or fibrotic phase followed by a stable (inactive) post-inflammatory phase. In the early, active inflammatory stages of TED, which are reported to typically last from up to 1 year (Bahn 2010 [NPL 8]) to 3 years (Douglas et al. 2022a [NPL 9]), studies have found both focal and diffuse inflammatory infiltration of the orbital tissues. Extraocular and levator muscles, and adipose tissues are infiltrated by mononuclear cells such as CD4+ and CD8+T lymphocytes, B cells and macrophages (Weetman et al. 1989 [NPL 10]; Gupta and Douglas 2011 [NPL 11]; Khong et al. 2016 [NPL 12]). T cells produce inflammatory cytokines such as IL-2 and IL-6, interferon-gamma and the tumor necrosis factors, activating the orbital fibroblasts which proliferate and produce collagen and glycosaminoglycans responsible for the tissue expansion seen in TED (Dik et al. 2016 [NPL 13]). In addition, orbital fibroblasts differentiate into new mature fat cells further increasing orbital tissue volume. In a disease of longer duration, the production of inflammatory mediators expands, as type 2 helper T cells predominate and produce IL-5 and IL-10. Other inflammatory mediators such as IL-1, IL-6, IL-16, and transforming growth factor beta [TGF-beta]) are produced with the involvement of macrophages, fibroblasts, and adipocytes in the orbit as well (Bahn 2012 [NPL 14]).

Historically, the mainstay of treatment for active, moderate-to-severe TED consists of off-label use of oral or IV pulses of corticosteroids (CS) (Kahaly et al. 2005 [NPL 15]; Bartalena et al. 2012 [NPL 16]). However, up to 40% of patients fail to respond to CS treatment, (Terwee et al. 2005 [NPL 17]; Zang et al. 2011 [NPL 18]). Many patients experience significant side effects including hyperglycemia, hypertension, gastritis and weight gain, or suffer relapse of disease after the CS treatment stops (Allen et al. 2021 [NPL 19]). Alternative therapies consist of orbital radiotherapy, and immunomodulatory therapy (rituximab, tocilizumab and azathioprine) used off-label in clinical practice.

Teprotumumab is currently approved for the treatment of TED in the United States. While it provides clinical improvement in some patients, adverse events such as elevated blood glucose, alopecia, nausea, fatigue, hearing impairment, muscle spasm, diarrhea and change in menstrual cycle present a substantial burden. Additionally, relapse rates after cessation of teprotumumab in patients with active TED are quite high with rebound of proptosis and diplopia in approximately 40% and 50%, respectively (Allen et al. 2021 [NPL 19]; Douglas et al. 2022b [NPL 20]). Due to the multiple side effects, limited efficacy to only a subset of patients, and relapse rates associated with the CS therapy as well as with teprotumumab, an unmet medical need remains in patients with moderate-to-severe TED for an alternative treatment option through disease modification accompanied by reduced risk of relapse and with an improved safety profile.

Furthermore, there is no established medical treatment for chronic inactive TED and surgical intervention remains the mainstay of treating patients who continue to suffer from visual impairment, diplopia or disfiguring proptosis and keratopathy, with patients needing to wait at least 6 months or longer after reaching the inactive phase of their disease before having any reconstructive or rehabilitative procedures (Bartalena et al. 2016 [NPL 21]). These surgeries are often performed 2 to 6 months apart (Bartalena 2013b [NPL 22]), suggesting that patients may suffer the physical, psychological, and financial burden of multiple surgeries over a number of years (Ponto et al. 2013 [NPL 23]). Surgical procedures are often unsuccessful, leaving the patient with residual visual impairments such as diplopia (Barrio-Barrio et al. 2015 [NPL 3]). Furthermore, up to 11% of patients undergoing orbital decompression require repeat procedures (Zhang Nunes et al. 2015 [NPL 24]).

Therefore, a new treatment option with a potentially disease modifying mechanism of action with long-term efficacy and with tolerable side effects administered via a convenient route of administration, is needed to address the needs of the TED patient population.

Recently, clinical trial results from academic investigator led studies and series with systemic administration of anti-IL6R antibody, tocilizumab, support an important role of IL-6 and its receptor in TED (Perez-Moreiras et al. 2014 [NPL 25]; Perez Moreiras et al. 2018 [NPL 26]; Sanchez-Bilbao et al. 2020 [NPL 27]).

Humanized antibodies like tocilizumab are first-generation antibody drugs. By improving first-generation antibody drugs, second-generation antibody drugs with improved efficacy, convenience, and cost are being developed [PTL 1 and PTL 2]. Among the second-generation antibody drugs is satralizumab (SA237), which is a novel anti-IL-6 receptor antibody to which improvement technologies such as enhancement of antigen-binding ability, pharmacokinetics, and stability, and reduction of immunogenicity risk, have been applied [PTL 2 and PTL 3].

Satralizumab is a humanized anti-IL-6 receptor monoclonal antibody with pH-dependent antigen binding. It specifically targets the human IL-6 receptor (IL-6R) and suppresses IL-6 signaling by inhibiting the binding of IL-6 to membrane-bound IL-6R and soluble IL-6R. Satralizumab was constructed by modifying the amino acid sequence of tocilizumab to prolong its plasma half-life. Satralizumab also shows a decreased antibody molecule isoelectric point and stronger binding to FcRn compared to tocilizumab. Moreover, its Fc region has been modified to minimize the antibody-dependent cellular cytotoxicity and complement-dependent cytotoxic effector activity compared to tocilizumab.

Prior-art literature information related to the disclosure of the present application is shown below.

Satralizumab has demonstrated efficacy and safety and is indicated for another autoantibody-mediated disease (i.e., NMOSD) as monotherapy or as an add-on to immunosuppressive therapy (IST; i.e., oral corticosteroid [OCS], azathioprine, or mycophenolate mofetil). The double-blind period of the Phase III studies in NMOSD included a total of 178 participants. Of the 178 participants, 104 participants were treated with a satralizumab dose of 120 mg subcutaneously every 4 weeks (Q4W) and 74 participants with placebo. Overall, satralizumab as monotherapy or in combination with IST was well tolerated by participants with NMOSD.

### CITATION LIST

### Patent Literature

[PTL 1] WO2009/041621
[PTL 2] WO2010/035769
[PTL 3] WO2016/136933

### Non-Patent Literature

[NPL 1] Bartley G B, Trans Am Ophthalmol Soc. 1994; 92:477-588.
[NPL 2] Wiersinga W M, Bartalena L, Thyroid. 2002; 12(10):855-60.
[NPL 3] Barrio-Barrio J, Sabater A L, Bonet-Farriol E, et al., J Ophthalmol. 2015; 2015:249125.
[NPL 4] Gerding M N, Terwee C B, Dekker F W, et al., Thyroid. 1997; 7(6):885-9.
[NPL 5] Kahaly G J, Hardt J, Petrak F, et al., Thyroid. 2002; 12(3):237-9.
[NPL 6] Wiersinga W M, Best Pract Res Clin Endocrinol Metab. 2012; 26:359-70.
[NPL 7] Ezra D G, Krell J, Rose G E, et al., J Clin Pathol. 2012; 65(7):608-13.
[NPL 8] Bahn R S, N Engl J Med. 2010; 362(8):726-38.
[NPL 9] Douglas R S, Kossler A L, Abrams J, et al., J Neuroophthalmol. 2022a; 42(3):334-9.
[NPL 10] Weetman A P, Cohen S, Gatter K C, et al., Clin Exp Immunol. 1989; 75(2):222-7.
[NPL 11] Gupta S, Douglas R S, Curr Opin Ophthalmol 2011; 22:385-90.
[NPL 12] Khong J J, McNab A A, Ebeling P R, et al., Br J Ophthalmol. 2016; 100(1):142-50.
[NPL 13] Dik W A, Virakul S, van Steensel L, Exp Eye Res. 2016; 142:83-91.
[NPL 14] Bahn R S. Autoimmunity and Graves' disease. Clin Pharmacol Ther. 2012; 91(4):577-9.
[NPL 15] Kahaly G J, Pitz S, Hommel G, et al., J Clin Endocrinol Metab. 2005; 90(9):5234-40.
[NPL 16] Bartalena L, Krassas G E, Wiersinga W, et al., J Clin Endocrinol Metab. 2012; 97(12):4454-63.
[NPL 17] Terwee C B, Prummel M F, Gerding M N, et al., Clin Endocrinol (Oxf). 2005; 62(2):145-55.
[NPL 18] Zang S, Ponto K A, Kahaly G J, J Clin Endocrinol Metab. 2011; 96(2):320-32.
[NPL 19] Allen R C, Bradley E A, Fante, R G, et al., Ophthalmology. 2021; 128(8):1125-8.
[NPL 20] Douglas R S, Dailey R, Subramanian P S, et al., JAMA Ophthalmol. 2022b; 140(4):328-35.
[NPL 21] Bartalena L, Baldeschi L, Boboridis K, et al., Eur Thyroid J. 2016; 5(1):9-26.
[NPL 22] Bartalena L, Eur Thyroid J. 2013b; 2(4):259-69.
[NPL 23] Ponto K A, Merkesdal S, Hommel G, et al., J Clin Endocrinol Metab. 2013; 98(1):145-52.
[NPL 24] Zhang-Nunes S X, Dang S, Gameau H C, et al., Orbit. 2015; 34(2):57-65.
[NPL 25] Perez-Moreiras J V, Alvarez-Lopez A, Gomez E C, Ophthalmic Plast Reconstr Surg. 2014; 30(2):162-167.
[NPL 26] Perez-Moreiras J V, Gomez-Reino J J, Maneiro J R, et al., Am J Ophthalmol. 2018; 195:181-90.
[NPL 27] Sanchez-Bilbao L, Martinez-Lopez D, Revenga M, et al., J Clin Med. 2020; 9(9):2816.

### SUMMARY OF INVENTION

### Technical Problem

The current main treatment for active TED is off-label use of corticosteroids, orbital radiotherapy and immunomodulatory therapy; chronic inactive TED is largely managed by surgical intervention. Teprotumumab, a monoclonal IGF-1R inhibitor, is approved for treatment of TED in the United States; however, it has been associated with several side effects and a high risk of relapse. There remains an unmet need for a treatment option for a disease modification therapy, with an improved safety profile and reduced risk of relapse.

### Solution to Problem

To solve the above-mentioned problem, the inventors designed Phase III, randomized, double-masked, placebo-controlled, multicenter study to evaluate the efficacy, safety, pharmacokinetics, and pharmacodynamics of satralizumab compared with placebo for the treatment of patients with moderate-to-severe active and chronic inactive TED. The study demonstrates that satralizumab addresses the above noted unmet needs, and also establishes the effectiveness of satralizumab in patients with TED for treating or preventing TED.

The present disclosure includes but not limited to the embodiments as exemplarily described below.

[A 1.1] A medicament for treating or preventing thyroid eye disease (TED) in a subject, comprising an IL-6 inhibitor as an active ingredient.

[A 1.2] The medicament of A1.1, wherein the IL-6 inhibitor is an anti-IL-6 antibody or antigen-binding fragment thereof, or an anti-IL-6 receptor antibody or antigen binding fragment thereof.

[A 1.3] The medicament of A1.1 or A1.2, wherein the IL-6 inhibitor is an anti-IL-6 receptor antibody or antigen binding fragment thereof.

[A1.4] The medicament of any one of A1.1-A1.3, wherein the IL-6 inhibitor is a humanized antibody.

[A1.5] The medicament of any one of A1.1-A1.4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.

[A1.6] The medicament of A1.5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.

[A1.7] The medicament of A1.5 or A1.6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.

[A1.8] The medicament of any one of A1.5-A1.7, wherein the IL-6 inhibitor is satralizumab.

[A1.9] The medicament of any one of A1.1-A1.8, preventing TED comprises reducing a risk of relapse of, delaying relapse of, reducing a frequency of relapse of, reducing severity of relapse of, or reducing the need for rescue therapy for relapse of TED in the subject.

[A1.10] The medicament of any one of A1.1-A1.9, wherein the subject has moderate-to-severe TED.

[A1.11] The medicament of any one of A1.1-A1.10, wherein the subject has active TED.

[A1.12] The medicament of any one of A1.1-A1.10, wherein the subject has chronic inactive TED.

[A1.13] The medicament of any one of A1.1-A1.12, wherein the subject is a current smoker.

[A1.14] The medicament of any one of A1.1-A1.12, wherein the subject is a former smoker.

[A1.15] The medicament of any one of A1.1-A1.12, wherein the subject has never smoked.

[A1.16] The medicament of any one of A1.1-A1.15, wherein the subject is positive for thyrotropin receptor (TSH receptor; TSHR) autoantibodies or insulin-like growth factor-1 receptor (IGF-1 receptor: IGF-1R) autoantibodies.

[A1.17] The medicament of any one of A1.1-A1.16, wherein the subject has experienced disease progression or relapse of the disease after the steroid treatment.

[A1.18] The medicament of any one of A1.1-A1.17, wherein the medicament is administered to the subject subcutaneously.

[A1.19] The medicament of any one of A1.5-A1.18, wherein the medicament is used such that 60 mg or 120 mg, 120 mg or 180 mg, and 180 mg or 240 mg of the anti-IL-6 receptor antibody or antigen binding fragment thereof is administered to the subject with body weight of less than 40 kg, between 40 and 100 kg, and over 100 kg respectively for each administration.

[A1.20] The medicament of any one of A1.5-A1.18, wherein the medicament is used such that 60 mg of the anti-IL-6 receptor antibody or antigen binding fragment thereof is administered to the subject with body weight of less than 40 kg for each administration.

[A1.21] The medicament of any one of A1.5-A1.18, wherein the medicament is used such that 120 mg of the anti-IL-6 receptor antibody or antigen binding fragment thereof is administered to the subject with body weight of less than 40 kg for each administration.

[A1.22] The medicament of any one of A1.5-A1.18, wherein the medicament is used such that 120 mg of the anti-IL-6 receptor antibody or antigen binding fragment thereof is administered to the subject with body weight of between 40 and 100 kg for each administration.

[A1.23] The medicament of any one of A1.5-A1.18, wherein the medicament is used such that 180 mg of the anti-IL-6 receptor antibody or antigen binding fragment thereof is administered to the subject with body weight of between 40 and 100 kg for each administration.

[A1.24] The medicament of any one of A1.5-A1.18, wherein the medicament is used such that 180 mg of the anti-IL-6 receptor antibody or antigen binding fragment thereof is administered to the subject with body weight of over 100 kg for each administration.

[A1.25] The medicament of any one of A1.5-A1.18, wherein the medicament is used such that 240 mg of the anti-IL-6 receptor antibody or antigen binding fragment thereof is administered to the subject with body weight of over 100 kg for each administration.

[A1.26] The medicament of any one of A1.5-A1.25, wherein the medicament is used such that the anti-IL-6 receptor antibody or antigen binding fragment thereof is administered to the subject two weeks after the medicament is previously administered to the subject.

[A1.27] The medicament of any one of A1.5-A1.25, wherein the medicament is used such that the anti-IL-6 receptor antibody or antigen binding fragment thereof is administered to the subject four weeks after the medicament is previously administered to the subject.

[A1.28] The medicament of any one of A1.5-A1.27, wherein the medicament is used such that the anti-IL-6 receptor antibody or antigen binding fragment thereof is administered to the subject every two weeks (Q2W) for three times, and thereafter every four weeks (Q4W).

[A1.29] The medicament of any one of A1.5-A1.28, wherein the medicament is administered to the subject at least seven times.

[A1.30] The medicament of any one of A1.1-A1.29, which
reduces or improves proptosis;
reduces or improves diplopia;
reduces or improves the Clinical Activity score (CAS);
improves extraocular motility;
reduces or improves motility-induced orbital pain;
reduces or improves spontaneous orbital pain;
improves the Visual Functioning and Appearance sub-scale scores of the Graves' Ophthalmopathy Quality of Life (GO-QoL);
improves the Ocular Surace Disease Index (registered trademark) (OSDI (registered trademark)) overall scores;
improves the OSDI Ocular Symptoms;
improves the OSDI Vision-related Function subscale scores; and/or
improves Oxford corneal staining scores in the subject.

[A1.31] The medicament of any one of A1.1-A1.30, which results in one or more of the followings in the subject:
2-mm or more reduction or improvement in proptosis;
1-grade or more reduction or improvement in diplopia;
2-point or more reduction or improvement in the CAS score;
an improvement in extraocular motility;
a reduction or improvement in motility-induced orbital pain;
a reduction or improvement in spontaneous orbital pain;
6-point or more improvement in the Visual Functioning and Appearance sub-scale scores of the GO-QoL;
10-point or more improvement in the OSDI overall scores;
an improvement in the OSDI Ocular Symptoms;
an improvement in the OSDI Vision-related Function subscale scores; or
an improvement in Oxford corneal staining scores.

[A2.1] A pharmaceutical composition for treating or preventing thyroid eye disease (TED) in a subject, comprising an IL-6 inhibitor as an active ingredient.

[A2.2] The pharmaceutical composition of A2.1, wherein the IL-6 inhibitor is an anti-IL-6 antibody or antigen-binding fragment thereof, or an anti-IL-6 receptor antibody or antigen binding fragment thereof.

[A2.3] The pharmaceutical composition of A2.1 or A2.2, wherein the IL-6 inhibitor is an anti-IL-6 receptor antibody or antigen binding fragment thereof.

[A2.4] The pharmaceutical composition of any one of A2.1-A2.3, wherein the IL-6 inhibitor is a humanized antibody.

[A2.5] The pharmaceutical composition of any one of A2.1-A2.4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.

[A2.6] The pharmaceutical composition of A2.5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.

[A2.7] The pharmaceutical composition of A2.5 or A2.6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.

[A2.8] The pharmaceutical composition of any one of A2.5-A2.7, wherein the IL-6 inhibitor is satralizumab.

[A2.9] The pharmaceutical composition of any one of A2.1-A2.8, preventing TED comprises reducing a risk of relapse of, delaying relapse of, reducing a frequency of relapse of, reducing severity of relapse of, or reducing the need for rescue therapy for relapse of TED in the subject.

[A2.10] The pharmaceutical composition of any one of A2.1-A2.9, wherein the subject has moderate-to-severe TED.

[A2.11] The pharmaceutical composition of any one of A2.1-A2.10, wherein the subject has active TED.

[A2.12] The pharmaceutical composition of any one of A2.1-A2.10, wherein the subject has chronic inactive TED.

[A2.13] The pharmaceutical composition of any one of A2.1-A2.12, wherein the subject is a current smoker.

[A2.14] The pharmaceutical composition of any one of A2.1-A2.12, wherein the subject is a former smoker.

[A2.15] The pharmaceutical composition of any one of A2.1-A2.12, wherein the subject has never smoked.

[A2.16] The pharmaceutical composition of any one of A2.1-A2.15, wherein the subject is positive for thyrotropin receptor (TSH receptor; TSHR) autoantibodies or insulin-like growth factor-1 receptor (IGF-1 receptor; IGF-1R) autoantibodies.

[A2.17] The pharmaceutical composition of any one of A2.1-A2.16, wherein the subject has experienced disease progression or relapse of the disease after the steroid treatment.

[A2.18] The pharmaceutical composition of any one of A2.1-A2.17, wherein the pharmaceutical composition is administered to the subject subcutaneously.

[A2.19] The pharmaceutical composition of any one of A2.5-A2.18, wherein the pharmaceutical composition is used such that 60 mg or 120 mg, 120 mg or 180 mg, and 180 mg or 240 mg of the anti-IL-6 receptor antibody or antigen binding fragment thereof is administered to the subject with body weight of less than 40 kg, between 40 and 100 kg, and over 100 kg respectively for each administration.

[A2.20] The pharmaceutical composition of any one of A2.5-A2.18, wherein the pharmaceutical composition is used such that 60 mg of the anti-IL-6 receptor antibody or antigen binding fragment thereof is administered to the subject with body weight of less than 40 kg for each administration.

[A2.21] The pharmaceutical composition of any one of A2.5-A2.18, wherein the pharmaceutical composition is used such that 120 mg of the anti-IL-6 receptor antibody or antigen binding fragment thereof is administered to the subject with body weight of less than 40 kg for each administration.

[A2.22] The pharmaceutical composition of any one of A2.5-A2.18, wherein the pharmaceutical composition is used such that 120 mg of the anti-IL-6 receptor antibody or antigen binding fragment thereof is administered to the subject with body weight of between 40 and 100 kg for each administration.

[A2.23] The pharmaceutical composition of any one of A2.5-A2.18, wherein the pharmaceutical composition is used such that 180 mg of the anti-IL-6 receptor antibody or antigen binding fragment thereof is administered to the subject with body weight of between 40 and 100 kg for each administration.

[A2.24] The pharmaceutical composition of any one of A2.5-A2.18, wherein the pharmaceutical composition is used such that 180 mg of the anti-IL-6 receptor antibody or antigen binding fragment thereof is administered to the subject with body weight of over 100 kg for each administration.

[A2.25] The pharmaceutical composition of any one of A2.5-A2.18, wherein the pharmaceutical composition is used such that 240 mg of the anti-IL-6 receptor antibody or antigen binding fragment thereof is administered to the subject with body weight of over 100 kg for each administration.

[A2.26] The pharmaceutical composition of any one of A2.5-A2.25, wherein the pharmaceutical composition is used such that the anti-IL-6 receptor antibody or antigen binding fragment thereof is administered to the subject two weeks after the pharmaceutical composition is previously administered to the subject.

[A2.27] The pharmaceutical composition of any one of A2.5-A2.25, wherein the pharmaceutical composition is used such that the anti-IL-6 receptor antibody or antigen binding fragment thereof is administered to the subject four weeks after the pharmaceutical composition is previously administered to the subject.

[A2.28] The pharmaceutical composition of any one of A2.5-A2.27, wherein the pharmaceutical composition is used such that the anti-IL-6 receptor antibody or antigen binding fragment thereof is administered to the subject every two weeks (Q2W) for three times, and thereafter every four weeks (Q4W).

[A2.29] The pharmaceutical composition of any one of A2.5-A2.28, wherein the pharmaceutical composition is administered to the subject at least seven times.

[A2.30] The pharmaceutical composition of any one of A2.1-A2.29, which
reduces or improves proptosis;
reduces or improves diplopia;
reduces or improves the CAS score;
improves extraocular motility;
reduces or improves motility-induced orbital pain;
reduces or improves spontaneous orbital pain;
improves the Visual Functioning and Appearance sub-scale scores of the GO-QoL;
improves the OSDI overall scores;
improves the OSDI Ocular Symptoms;
improves the OSDI Vision-related Function subscale scores; and/or
improves Oxford corneal staining scores in the subject.

[A2.31] The pharmaceutical composition of any one of A2.1-A2.30, which results in one or more of the followings in the subject:
2-mm or more reduction or improvement in proptosis;
1-grade or more reduction or improvement in diplopia;
2-point or more reduction or improvement in the CAS score;
an improvement in extraocular motility;
a reduction or improvement in motility-induced orbital pain;
a reduction or improvement in spontaneous orbital pain;
6-point or more improvement in the Visual Functioning and Appearance sub-scale scores of the GO-QoL;
10-point or more improvement in the OSDI overall scores;
an improvement in the OSDI Ocular Symptoms;
an improvement in the OSDI Vision-related Function subscale scores; or
an improvement in Oxford corneal staining scores.

[B1] Use of an IL-6 inhibitor in the preparation of a medicament for treating or preventing thyroid eye disease (TED) in a subject.

[B2] The use of B1, wherein the IL-6 inhibitor is an anti-IL-6 antibody or antigen-binding fragment thereof, or an anti-IL-6 receptor antibody or antigen binding fragment thereof.

[B3] The use of B1 or B2, wherein the IL-6 inhibitor is an anti-IL-6 receptor antibody or antigen binding fragment thereof.

[B4] The use of any one of B1-B3, wherein the IL-6 inhibitor is a humanized antibody.

[B5] The use of any one of B1-B4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.

[B6] The use of B5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.

[B7] The use of B5 or B6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.

[B8] The use of any one of B5-B7, wherein the IL-6 inhibitor is satralizumab.

[B9] The use of any one of B1-B8, wherein preventing TED comprises reducing a risk of relapse of, delaying relapse of, reducing a frequency of relapse of, reducing severity of relapse of, or reducing the need for rescue therapy for relapse of TED in the subject.

[B10] The use of any one of B1-B9, wherein the subject has moderate-to-severe TED.

[B11] The use of any one of B1-B10, wherein the subject has active TED.

[B12] The use of any one of B1-B10, wherein the subject has chronic inactive TED.

[B13] The use of any one of B1-B12, wherein the subject is a current smoker.

[B14] The use of any one of B1-B12, wherein the subject is a former smoker.

[B15] The use of any one of B1-B12, wherein the subject has never smoked.

[B16] The use of any one of B1-B15, wherein the subject is positive for thyrotropin receptor (TSH receptor; TSHR) autoantibodies or insulin-like growth factor-1 receptor (IGF-1 receptor; IGF-1R) autoantibodies.

[B17] The use of any one of B1-B16, wherein the subject has experienced disease progression or relapse of the disease after the steroid treatment.

[B18] The use of any one of B1-B17, wherein the medicament is administered to the subject subcutaneously.

[B19] The use of any one of B5-B18, wherein the medicament is used such that 60 mg or 120 mg, 120 mg or 180 mg, and 180 mg or 240 mg of the anti-IL-6 receptor antibody or antigen binding fragment thereof is administered to the subject with body weight of less than 40 kg, between 40 and 100 kg, and over 100 kg respectively for each administration.

[B20] The use of any one of B5-B18, wherein the medicament is used such that 60 mg of the anti-IL-6 receptor antibody or antigen binding fragment thereof is administered to the subject with body weight of less than 40 kg for each administration.

[B21] The use of any one of B5-B18, wherein the medicament is used such that 120 mg of the anti-IL-6 receptor antibody or antigen binding fragment thereof is administered to the subject with body weight of less than 40 kg for each administration.

[B22] The use of any one of B5-B18, wherein the medicament is used such that 120 mg of the anti-IL-6 receptor antibody or antigen binding fragment thereof is administered to the subject with body weight of between 40 and 100 kg for each administration.

[B23] The use of any one of B5-B18, wherein the medicament is used such that 180 mg of the anti-IL-6 receptor antibody or antigen binding fragment thereof is administered to the subject with body weight of between 40 and 100 kg for each administration.

[B24] The use of any one of B5-B18, wherein the medicament is used such that 180 mg of the anti-IL-6 receptor antibody or antigen binding fragment thereof is administered to the subject with body weight of over 100 kg for each administration.

[B25] The use of any one of B5-B18, wherein the medicament is used such that 240 mg of the anti-IL-6 receptor antibody or antigen binding fragment thereof is administered to the subject with body weight of over 100 kg for each administration.

[B26] The use of any one of B5-B25, wherein the medicament is used such that the anti-IL-6 receptor antibody or antigen binding fragment thereof is administered to the subject every two weeks (Q2W) for three times, and thereafter every four weeks (Q4W).

[B27] The use of any one of B5-B26, wherein the medicament is administered to the subject at least seven times.

[B28] The use of any one of B1-B27, wherein the medicament
reduces or improves proptosis;
reduces or improves diplopia;
reduces or improves the CAS score;
improves extraocular motility;
reduces or improves motility-induced orbital pain;
reduces or improves spontaneous orbital pain;
improves the Visual Functioning and Appearance sub-scale scores of the GO-QoL;
improves the OSDI overall scores;
improves the OSDI Ocular Symptoms;
improves the OSDI Vision-related Function subscale scores; and/or
improves Oxford corneal staining scores in the subject. [B29] The use of any one of B1-B28, wherein the medicament results in one or more of the followings in the subject:
2-mm or more reduction or improvement in proptosis;
1-grade or more reduction or improvement in diplopia;
2-point or more reduction or improvement in the CAS score;
an improvement in extraocular motility;
a reduction or improvement in motility-induced orbital pain;
a reduction or improvement in spontaneous orbital pain;
6-point or more improvement in the Visual Functioning and Appearance sub-scale scores of the GO-QoL;
10-point or more improvement in the OSDI overall scores;
an improvement in the OSDI Ocular Symptoms;
an improvement in the OSDI Vision-related Function subscale scores; or
an improvement in Oxford corneal staining scores.

[C1] An IL-6 inhibitor for use in treating or preventing thyroid eye disease (TED) in a subject.

[C2] The IL-6 inhibitor for use of C1, wherein the IL-6 inhibitor is an anti-IL-6 antibody or antigen-binding fragment thereof, or an anti-IL-6 receptor antibody or antigen binding fragment thereof.

[C3] The IL-6 inhibitor for use of C1 or C2, wherein the IL-6 inhibitor is an anti-IL-6 receptor antibody or antigen binding fragment thereof.

[C4] The IL-6 inhibitor for use of any one of C1-C3, wherein the IL-6 inhibitor is a humanized antibody.

[C5] The IL-6 inhibitor for use of any one of C1-C4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.

[C6] The IL-6 inhibitor for use of C5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.

[C7] The IL-6 inhibitor for use of C5 or C6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.

[C8] The IL-6 inhibitor for use of any one of C5-C7, wherein the IL-6 inhibitor is satralizumab.

[C9] The IL-6 inhibitor for use of any one of C1-C8, preventing TED comprises reducing a risk of relapse of, delaying relapse of, reducing a frequency of relapse of, reducing severity of relapse of, or reducing the need for rescue therapy for relapse of TED in the subject.

[C10] The IL-6 inhibitor for use of any one of C1-C9, wherein the subject has moderate-to-severe TED.

[C11] The IL-6 inhibitor for use of any one of C1-C10, wherein the subject has active TED.

[C12] The IL-6 inhibitor for use of any one of C1-C10, wherein the subject has chronic inactive TED.

[C13] The IL-6 inhibitor for use of any one of C1-C12, wherein the subject is a current smoker.

[C14] The IL-6 inhibitor for use of any one of C1-C12, wherein the subject is a former smoker.

[C15] The IL-6 inhibitor for use of any one of C1-C12, wherein the subject has never smoked.

[C16] The IL-6 inhibitor for use of any one of C1-C15, wherein the subject is positive for thyrotropin receptor (TSH receptor; TSHR) autoantibodies or insulin-like growth factor-1 receptor (IGF-1 receptor: IGF-1R) autoantibodies.

[C17] The IL-6 inhibitor for use of any one of C1-C16, wherein the subject has experienced disease progression or relapse of the disease after the steroid treatment.

[C18] The IL-6 inhibitor for use of any one of C1-C17, wherein the IL-6 inhibitor is administered to the subject subcutaneously.

[C19] The IL-6 inhibitor for use of any one of C5-C18, wherein 60 mg or 120 mg, 120 mg or 180 mg, and 180 mg or 240 mg of the anti-IL-6 receptor antibody or antigen binding fragment thereof is administered to the subject with body weight of less than 40 kg, between 40 and 100 kg, and over 100 kg respectively for each administration.

[C20] The IL-6 inhibitor for use of any one of C5-C18, wherein 60 mg of the anti-IL-6 receptor antibody or antigen binding fragment thereof is administered to the subject with body weight of less than 40 kg for each administration.

[C21] The IL-6 inhibitor for use of any one of C5-C18, wherein 120 mg of the anti-IL-6 receptor antibody or antigen binding fragment thereof is administered to the subject with body weight of less than 40 kg for each administration.

[C22] The IL-6 inhibitor for use of any one of C5-C18, wherein 120 mg of the antiIL-6 receptor antibody or antigen binding fragment thereof is administered to the subject with body weight of between 40 and 100 kg for each administration.

[C23] The IL-6 inhibitor for use of any one of C5-C18, wherein 180 mg of the antiIL-6 receptor antibody or antigen binding fragment thereof is administered to the subject with body weight of between 40 and 100 kg for each administration.

[C24] The IL-6 inhibitor for use of any one of C5-C18, wherein 180 mg of the antiIL-6 receptor antibody or antigen binding fragment thereof is administered to the subject with body weight of over 100 kg for each administration.

[C25] The IL-6 inhibitor for use of any one of C5-C18, wherein 240 mg of the antiIL-6 receptor antibody or antigen binding fragment thereof is administered to the subject with body weight of over 100 kg for each administration.

[C26] The IL-6 inhibitor for use any one of C5-C25, wherein the IL-6 inhibitor for use is used such that the anti-IL-6 receptor antibody or antigen binding fragment thereof is administered to the subject two weeks after the IL-6 inhibitor is previously administered to the subject.

[C27] The IL-6 inhibitor for use of any one of C5-C25, wherein the IL-6 inhibitor for use is used such that the anti-IL-6 receptor antibody or antigen binding fragment thereof is administered to the subject four weeks after the IL-6 inhibitor is previously administered to the subject.

[C28] The IL-6 inhibitor for use of any one of C5-C27, wherein the anti-IL-6 receptor antibody or antigen binding fragment thereof is administered to the subject every two weeks (Q2W) for three times, and thereafter every four weeks (Q4W).

[C29] The IL-6 inhibitor for use of any one of C5-C28, wherein the anti-IL-6 receptor antibody or antigen binding fragment thereof is administered to the subject at least seven times.

[C30] The IL-6 inhibitor for use of any one of C1-C29, wherein the IL-6 inhibitor
reduces or improves proptosis;
reduces or improves diplopia;
reduces or improves the CAS score;
improves extraocular motility;
reduces or improves motility-induced orbital pain;
reduces or improves spontaneous orbital pain;
improves the Visual Functioning and Appearance sub-scale scores of the GO-QoL;
improves the OSDI overall scores;
improves the OSDI Ocular Symptoms;
improves the OSDI Vision-related Function subscale scores; and/or
improves Oxford corneal staining scores in the subject.

[C31] The IL-6 inhibitor for use of any one of C1-C30, wherein the IL-6 inhibitor results in one or more of the followings in the subject:
2-mm or more reduction or improvement in proptosis;
1-grade or more reduction or improvement in diplopia;
2-point or more reduction or improvement in the CAS score;
an improvement in extraocular motility;
a reduction or improvement in motility-induced orbital pain;
a reduction or improvement in spontaneous orbital pain;
6-point or more improvement in the Visual Functioning and Appearance sub-scale scores of the GO-QoL;
10-point or more improvement in the OSDI overall scores;
an improvement in the OSDI Ocular Symptoms;
an improvement in the OSDI Vision-related Function subscale scores; or
an improvement in Oxford corneal staining scores.

[C2.1] Satralizumab for use in treating thyroid eye disease (TED) in a subject.

[C2.2] Satralizumab for use in treating thyroid eye disease (TED) in a subject; wherein 60 mg of satralizumab for each administration is administered subcutaneously to the subject with body weight of less than 40 kg every two weeks (Q2W) for three times, and thereafter every four weeks (Q4W).

[C2.3] Satralizumab for use in treating thyroid eye disease (TED) in a subject; wherein 120 mg of satralizumab for each administration is administered subcutaneously to the subject with body weight of less than 40 kg every two weeks (Q2W) for three times, and thereafter every four weeks (Q4W).

[C2.4] Satralizumab for use in treating thyroid eye disease (TED) in a subject; wherein 120 mg of satralizumab for each administration is administered subcutaneously to the subject with body weight of between 40 and 100 kg every two weeks (Q2W) for three times, and thereafter every four weeks (Q4W).

[C2.5] Satralizumab for use in treating thyroid eye disease (TED) in a subject; wherein 180 mg of satralizumab for each administration is administered subcutaneously to the subject with body weight of between 40 and 100 kg every two weeks (Q2W) for three times, and thereafter every four weeks (Q4W).

[C2.6] Satralizumab for use in treating thyroid eye disease (TED) in a subject; wherein 180 mg of satralizumab for each administration is administered subcutaneously to the subject with body weight of over 100 kg every two weeks (Q2W) for three times, and thereafter every four weeks (Q4W).

[C2.7] Satralizumab for use in treating thyroid eye disease (TED) in a subject; wherein 240 mg of satralizumab for each administration is administered subcutaneously to the subject with body weight of over 100 kg every two weeks (Q2W) for three times, and thereafter every four weeks (Q4W).

[C2.8] Satralizumab for use in treating moderate-to-severe active thyroid eye disease (TED) in a subject.

[C2.9] Satralizumab for use in treating moderate-to-severe active thyroid eye disease (TED) in a subject; wherein 60 mg of satralizumab for each administration is administered subcutaneously to the subject with body weight of less than 40 kg every two weeks (Q2W) for three times, and thereafter every four weeks (Q4W).

[C2.10] Satralizumab for use in treating moderate-to-severe active thyroid eye disease (TED) in a subject; wherein 120 mg of satralizumab for each administration is administered subcutaneously to the subject with body weight of less than 40 kg every two weeks (Q2W) for three times, and thereafter every four weeks (Q4W).

[C2.11] Satralizumab for use in treating moderate-to-severe active thyroid eye disease (TED) in a subject; wherein 120 mg of satralizumab for each administration is administered subcutaneously to the subject with body weight of between 40 and 100 kg every two weeks (Q2W) for three times, and thereafter every four weeks (Q4W).

[C2.12] Satralizumab for use in treating moderate-to-severe active thyroid eye disease (TED) in a subject; wherein 180 mg of satralizumab for each administration is administered subcutaneously to the subject with body weight of between 40 and 100 kg every two weeks (Q2W) for three times, and thereafter every four weeks (Q4W).

[C2.13] Satralizumab for use in treating moderate-to-severe active thyroid eye disease (TED) in a subject; wherein 180 mg of satralizumab for each administration is administered subcutaneously to the subject with body weight of over 100 kg every two weeks (Q2W) for three times, and thereafter every four weeks (Q4W).

[C2.14] Satralizumab for use in treating moderate-to-severe active thyroid eye disease (TED) in a subject; wherein 240 mg of satralizumab for each administration is administered subcutaneously to the subject with body weight of over 100 kg every two weeks (Q2W) for three times, and thereafter every four weeks (Q4W).

[C2.15] Satralizumab for use in treating chronic inactive thyroid eye disease (TED) in a subject.

[C2.16] Satralizumab for use in treating chronic inactive thyroid eye disease (TED) in a subject; wherein 60 mg of satralizumab for each administration is administered subcutaneously to the subject with body weight of less than 40 kg every two weeks (Q2W) for three times, and thereafter every four weeks (Q4W).

[C2.17] Satralizumab for use in treating chronic inactive thyroid eye disease (TED) in a subject; wherein 120 mg of satralizumab for each administration is administered subcutaneously to the subject with body weight of less than 40 kg every two weeks (Q2W) for three times, and thereafter every four weeks (Q4W).

[C2.18] Satralizumab for use in treating chronic inactive thyroid eye disease (TED) in a subject; wherein 120 mg of satralizumab for each administration is administered subcutaneously to the subject with body weight of between 40 and 100 kg every two weeks (Q2W) for three times, and thereafter every four weeks (Q4W).

[C2.19] Satralizumab for use in treating chronic inactive thyroid eye disease (TED) in a subject; wherein 180 mg of satralizumab for each administration is administered subcutaneously to the subject with body weight of between 40 and 100 kg every two weeks (Q2W) for three times, and thereafter every four weeks (Q4W).

[C2.20] Satralizumab for use in treating chronic inactive thyroid eye disease (TED) in a subject; wherein 180 mg of satralizumab for each administration is administered subcutaneously to the subject with body weight of over 100 kg every two weeks (Q2W) for three times, and thereafter every four weeks (Q4W).

[C2.21] Satralizumab for use in treating chronic inactive thyroid eye disease (TED) in a subject; wherein 240 mg of satralizumab for each administration is administered subcutaneously to the subject with body weight of over 100 kg every two weeks (Q2W) for three times, and thereafter every four weeks (Q4W).

[C2.22] Satralizumab for use in treating thyroid eye disease (TED) in a subject, wherein the subject is a current smoker.

[C2.23] Satralizumab for use in treating thyroid eye disease (TED) in a subject, wherein the subject is a current smoker; and wherein 60 mg of satralizumab for each administration is administered subcutaneously to the subject with body weight of less than 40 kg every two weeks (Q2W) for three times, and thereafter every four weeks (Q4W).

[C2.24] Satralizumab for use in treating thyroid eye disease (TED) in a subject, wherein the subject is a current smoker; and wherein 120 mg of satralizumab for each administration is administered subcutaneously to the subject with body weight of less than 40 kg every two weeks (Q2W) for three times, and thereafter every four weeks (Q4W).

[C2.25] Satralizumab for use in treating thyroid eye disease (TED) in a subject, wherein the subject is a current smoker; and wherein 120 mg of satralizumab for each administration is administered subcutaneously to the subject with body weight of between 40 and 100 kg every two weeks (Q2W) for three times, and thereafter every four weeks (Q4W).

[C2.26] Satralizumab for use in treating thyroid eye disease (TED) in a subject, wherein the subject is a current smoker; and wherein 180 mg of satralizumab for each administration is administered subcutaneously to the subject with body weight of between 40 and 100 kg every two weeks (Q2W) for three times, and thereafter every four weeks (Q4W).

[C2.27] Satralizumab for use in treating thyroid eye disease (TED) in a subject, wherein the subject is a current smoker; and wherein 180 mg of satralizumab for each administration is administered subcutaneously to the subject with body weight of over 100 kg every two weeks (Q2W) for three times, and thereafter every four weeks (Q4W).

[C2.28] Satralizumab for use in treating thyroid eye disease (TED) in a subject, wherein the subject is a current smoker; and wherein 240 mg of satralizumab for each administration is administered subcutaneously to the subject with body weight of over 100 kg every two weeks (Q2W) for three times, and thereafter every four weeks (Q4W).

[C2.29] Satralizumab for use in treating moderate-to-severe active thyroid eye disease (TED) in a subject, wherein the subject is a current smoker.

[C2.30] Satralizumab for use in treating moderate-to-severe active thyroid eye disease (TED) in a subject, wherein the subject is a current smoker; and wherein 60 mg of satralizumab for each administration is administered subcutaneously to the subject with body weight of less than 40 kg every two weeks (Q2W) for three times, and thereafter every four weeks (Q4W).

[C2.31] Satralizumab for use in treating moderate-to-severe active thyroid eye disease (TED) in a subject, wherein the subject is a current smoker; and wherein 120 mg of satralizumab for each administration is administered subcutaneously to the subject with body weight of less than 40 kg every two weeks (Q2W) for three times, and thereafter every four weeks (Q4W).

[C2.32] Satralizumab for use in treating moderate-to-severe active thyroid eye disease (TED) in a subject, wherein the subject is a current smoker; and wherein 120 mg of satralizumab for each administration is administered subcutaneously to the subject with body weight of between 40 and 100 kg every two weeks (Q2W) for three times, and thereafter every four weeks (Q4W).

[C2.33] Satralizumab for use in treating moderate-to-severe active thyroid eye disease (TED) in a subject, wherein the subject is a current smoker; and wherein 180 mg of satralizumab for each administration is administered subcutaneously to the subject with body weight of between 40 and 100 kg every two weeks (Q2W) for three times, and thereafter every four weeks (Q4W).

[C2.34] Satralizumab for use in treating moderate-to-severe active thyroid eye disease (TED) in a subject, wherein the subject is a current smoker; and wherein 180 mg of satralizumab for each administration is administered subcutaneously to the subject with body weight of over 100 kg every two weeks (Q2W) for three times, and thereafter every four weeks (Q4W).

[C2.35] Satralizumab for use in treating moderate-to-severe active thyroid eye disease (TED) in a subject, wherein the subject is a current smoker; and wherein 240 mg of satralizumab for each administration is administered subcutaneously to the subject with body weight of over 100 kg every two weeks (Q2W) for three times, and thereafter every four weeks (Q4W).

[C2.36] Satralizumab for use in treating chronic inactive thyroid eye disease (TED) in a subject, wherein the subject is a current smoker.

[C2.37] Satralizumab for use in treating chronic inactive thyroid eye disease (TED) in a subject, wherein the subject is a current smoker; and wherein 60 mg of satralizumab for each administration is administered subcutaneously to the subject with body weight of less than 40 kg every two weeks (Q2W) for three times, and thereafter every four weeks (Q4W).

[C2.38] Satralizumab for use in treating chronic inactive thyroid eye disease (TED) in a subject, wherein the subject is a current smoker; and wherein 120 mg of satralizumab for each administration is administered subcutaneously to the subject with body weight of less than 40 kg every two weeks (Q2W) for three times, and thereafter every four weeks (Q4W).

[C2.39] Satralizumab for use in treating chronic inactive thyroid eye disease (TED) in a subject, wherein the subject is a current smoker; and wherein 120 mg of satralizumab for each administration is administered subcutaneously to the subject with body weight of between 40 and 100 kg every two weeks (Q2W) for three times, and thereafter every four weeks (Q4W).

[C2.40] Satralizumab for use in treating chronic inactive thyroid eye disease (TED) in a subject, wherein the subject is a current smoker; and wherein 180 mg of satralizumab for each administration is administered subcutaneously to the subject with body weight of between 40 and 100 kg every two weeks (Q2W) for three times, and thereafter every four weeks (Q4W).

[C2.41] Satralizumab for use in treating chronic inactive thyroid eye disease (TED) in a subject, wherein the subject is a current smoker; and wherein 180 mg of satralizumab for each administration is administered subcutaneously to the subject with body weight of over 100 kg every two weeks (Q2W) for three times, and thereafter every four weeks (Q4W).

[C2.42] Satralizumab for use in treating chronic inactive thyroid eye disease (TED) in a subject, wherein the subject is a current smoker; and wherein 240 mg of satralizumab for each administration is administered subcutaneously to the subject with body weight of over 100 kg every two weeks (Q2W) for three times, and thereafter every four weeks (Q4W).

[C2.43] Satralizumab for use in treating thyroid eye disease (TED) in a subject, wherein the subject is a former smoker.

[C2.44] Satralizumab for use in treating thyroid eye disease (TED) in a subject, wherein the subject is a former smoker; and wherein 60 mg of satralizumab for each administration is administered subcutaneously to the subject with body weight of less than 40 kg every two weeks (Q2W) for three times, and thereafter every four weeks (Q4W).

[C2.45] Satralizumab for use in treating thyroid eye disease (TED) in a subject, wherein the subject is a former smoker; and wherein 120 mg of satralizumab for each administration is administered subcutaneously to the subject with body weight of less than 40 kg every two weeks (Q2W) for three times, and thereafter every four weeks (Q4W).

[C2.46] Satralizumab for use in treating thyroid eye disease (TED) in a subject, wherein the subject is a former smoker; and wherein 120 mg of satralizumab for each administration is administered subcutaneously to the subject with body weight of between 40 and 100 kg every two weeks (Q2W) for three times, and thereafter every four weeks (Q4W).

[C2.47] Satralizumab for use in treating thyroid eye disease (TED) in a subject, wherein the subject is a former smoker; and wherein 180 mg of satralizumab for each administration is administered subcutaneously to the subject with body weight of between 40 and 100 kg every two weeks (Q2W) for three times, and thereafter every four weeks (Q4W).

[C2.48] Satralizumab for use in treating thyroid eye disease (TED) in a subject, wherein the subject is a former smoker; and wherein 180 mg of satralizumab for each administration is administered subcutaneously to the subject with body weight of over 100 kg every two weeks (Q2W) for three times, and thereafter every four weeks (Q4W).

[C2.49] Satralizumab for use in treating thyroid eye disease (TED) in a subject, wherein the subject is a former smoker; and wherein 240 mg of satralizumab for each administration is administered subcutaneously to the subject with body weight of over 100 kg every two weeks (Q2W) for three times, and thereafter every four weeks (Q4W).

[C2.50] Satralizumab for use in treating moderate-to-severe active thyroid eye disease (TED) in a subject, wherein the subject is a former smoker.

[C2.51] Satralizumab for use in treating moderate-to-severe active thyroid eye disease (TED) in a subject, wherein the subject is a former smoker; and wherein 60 mg of satralizumab for each administration is administered subcutaneously to the subject with body weight of less than 40 kg every two weeks (Q2W) for three times, and thereafter every four weeks (Q4W).

[C2.52] Satralizumab for use in treating moderate-to-severe active thyroid eye disease (TED) in a subject, wherein the subject is a former smoker; and wherein 120 mg of satralizumab for each administration is administered subcutaneously to the subject with body weight of less than 40 kg every two weeks (Q2W) for three times, and thereafter every four weeks (Q4W).

[C2.53] Satralizumab for use in treating moderate-to-severe active thyroid eye disease (TED) in a subject, wherein the subject is a former smoker; and wherein 120 mg of satralizumab for each administration is administered subcutaneously to the subject with body weight of between 40 and 100 kg every two weeks (Q2W) for three times, and thereafter every four weeks (Q4W).

[C2.54] Satralizumab for use in treating moderate-to-severe active thyroid eye disease (TED) in a subject, wherein the subject is a former smoker; and wherein 180 mg of satralizumab for each administration is administered subcutaneously to the subject with body weight of between 40 and 100 kg every two weeks (Q2W) for three times, and thereafter every four weeks (Q4W).

[C2.55] Satralizumab for use in treating moderate-to-severe active thyroid eye disease (TED) in a subject, wherein the subject is a former smoker; and wherein 180 mg of satralizumab for each administration is administered subcutaneously to the subject with body weight of over 100 kg every two weeks (Q2W) for three times, and thereafter every four weeks (Q4W).

[C2.56] Satralizumab for use in treating moderate-to-severe active thyroid eye disease (TED) in a subject, wherein the subject is a former smoker; and wherein 240 mg of satralizumab for each administration is administered subcutaneously to the subject with body weight of over 100 kg every two weeks (Q2W) for three times, and thereafter every four weeks (Q4W).

[C2.57] Satralizumab for use in treating chronic inactive thyroid eye disease (TED) in a subject, wherein the subject is a former smoker.

[C2.58] Satralizumab for use in treating chronic inactive thyroid eye disease (TED) in a subject, wherein the subject is a former smoker; and wherein 60 mg of satralizumab for each administration is administered subcutaneously to the subject with body weight of less than 40 kg every two weeks (Q2W) for three times, and thereafter every four weeks (Q4W).

[C2.59] Satralizumab for use in treating chronic inactive thyroid eye disease (TED) in a subject, wherein the subject is a former smoker; and wherein 120 mg of satralizumab for each administration is administered subcutaneously to the subject with body weight of less than 40 kg every two weeks (Q2W) for three times, and thereafter every four weeks (Q4W).

[C2.60] Satralizumab for use in treating chronic inactive thyroid eye disease (TED) in a subject, wherein the subject is a former smoker; and wherein 120 mg of satralizumab for each administration is administered subcutaneously to the subject with body weight of between 40 and 100 kg every two weeks (Q2W) for three times, and thereafter every four weeks (Q4W).

[C2.61] Satralizumab for use in treating chronic inactive thyroid eye disease (TED) in a subject, wherein the subject is a former smoker; and wherein 180 mg of satralizumab for each administration is administered subcutaneously to the subject with body weight of between 40 and 100 kg every two weeks (Q2W) for three times, and thereafter every four weeks (Q4W).

[C2.62] Satralizumab for use in treating chronic inactive thyroid eye disease (TED) in a subject, wherein the subject is a former smoker; and wherein 180 mg of satralizumab for each administration is administered subcutaneously to the subject with body weight of over 100 kg every two weeks (Q2W) for three times, and thereafter every four weeks (Q4W).

[C2.63] Satralizumab for use in treating chronic inactive thyroid eye disease (TED) in a subject, wherein the subject is a former smoker; and wherein 240 mg of satralizumab for each administration is administered subcutaneously to the subject with body weight of over 100 kg every two weeks (Q2W) for three times, and thereafter every four weeks (Q4W).

[C2.64] Satralizumab for use in treating thyroid eye disease (TED) in a subject, wherein the subject has never smoked.

[C2.65] Satralizumab for use in treating thyroid eye disease (TED) in a subject, wherein the subject has never smoked; and wherein 60 mg of satralizumab for each administration is administered subcutaneously to the subject with body weight of less than 40 kg every two weeks (Q2W) for three times, and thereafter every four weeks (Q4W).

[C2.66] Satralizumab for use in treating thyroid eye disease (TED) in a subject, wherein the subject has never smoked; and wherein 120 mg of satralizumab for each administration is administered subcutaneously to the subject with body weight of less than 40 kg every two weeks (Q2W) for three times, and thereafter every four weeks (Q4W).

[C2.67] Satralizumab for use in treating thyroid eye disease (TED) in a subject, wherein the subject has never smoked; and wherein 120 mg of satralizumab for each administration is administered subcutaneously to the subject with body weight of between 40 and 100 kg every two weeks (Q2W) for three times, and thereafter every four weeks (Q4W).

[C2.68] Satralizumab for use in treating thyroid eye disease (TED) in a subject, wherein the subject has never smoked; and wherein 180 mg of satralizumab for each administration is administered subcutaneously to the subject with body weight of between 40 and 100 kg every two weeks (Q2W) for three times, and thereafter every four weeks (Q4W).

[C2.69] Satralizumab for use in treating thyroid eye disease (TED) in a subject, wherein the subject has never smoked; and wherein 180 mg of satralizumab for each administration is administered subcutaneously to the subject with body weight of over 100 kg every two weeks (Q2W) for three times, and thereafter every four weeks (Q4W).

[C2.70] Satralizumab for use in treating thyroid eye disease (TED) in a subject, wherein the subject has never smoked; and wherein 240 mg of satralizumab for each administration is administered subcutaneously to the subject with body weight of over 100 kg every two weeks (Q2W) for three times, and thereafter every four weeks (Q4W).

[C2.71] Satralizumab for use in treating moderate-to-severe active thyroid eye disease (TED) in a subject, wherein the subject has never smoked.

[C2.72] Satralizumab for use in treating moderate-to-severe active thyroid eye disease (TED) in a subject, wherein the subject has never smoked; and wherein 60 mg of satralizumab for each administration is administered subcutaneously to the subject with body weight of less than 40 kg every two weeks (Q2W) for three times, and thereafter every four weeks (Q4W).

[C2.73] Satralizumab for use in treating moderate-to-severe active thyroid eye disease (TED) in a subject, wherein the subject has never smoked; and wherein 120 mg of satralizumab for each administration is administered subcutaneously to the subject with body weight of less than 40 kg every two weeks (Q2W) for three times, and thereafter every four weeks (Q4W).

[C2.74] Satralizumab for use in treating moderate-to-severe active thyroid eye disease (TED) in a subject, wherein the subject has never smoked; and wherein 120 mg of satralizumab for each administration is administered subcutaneously to the subject with body weight of between 40 and 100 kg every two weeks (Q2W) for three times, and thereafter every four weeks (Q4W).

[C2.75] Satralizumab for use in treating moderate-to-severe active thyroid eye disease (TED) in a subject, wherein the subject has never smoked; and wherein 180 mg of satralizumab for each administration is administered subcutaneously to the subject with body weight of between 40 and 100 kg every two weeks (Q2W) for three times, and thereafter every four weeks (Q4W).

[C2.76] Satralizumab for use in treating moderate-to-severe active thyroid eye disease (TED) in a subject, wherein the subject has never smoked; and wherein 180 mg of satralizumab for each administration is administered subcutaneously to the subject with body weight of over 100 kg every two weeks (Q2W) for three times, and thereafter every four weeks (Q4W).

[C2.77] Satralizumab for use in treating moderate-to-severe active thyroid eye disease (TED) in a subject, wherein the subject has never smoked; and wherein 240 mg of satralizumab for each administration is administered subcutaneously to the subject with body weight of over 100 kg every two weeks (Q2W) for three times, and thereafter every four weeks (Q4W).

[C2.78] Satralizumab for use in treating chronic inactive thyroid eye disease (TED) in a subject, wherein the subject has never smoked.

[C2.79] Satralizumab for use in treating chronic inactive thyroid eye disease (TED) in a subject, wherein the subject has never smoked; and wherein 60 mg of satralizumab for each administration is administered subcutaneously to the subject with body weight of less than 40 kg every two weeks (Q2W) for three times, and thereafter every four weeks (Q4W).

[C2.80] Satralizumab for use in treating chronic inactive thyroid eye disease (TED) in a subject, wherein the subject has never smoked; and wherein 120 mg of satralizumab for each administration is administered subcutaneously to the subject with body weight of less than 40 kg every two weeks (Q2W) for three times, and thereafter every four weeks (Q4W).

[C2.81] Satralizumab for use in treating chronic inactive thyroid eye disease (TED) in a subject, wherein the subject has never smoked; and wherein 120 mg of satralizumab for each administration is administered subcutaneously to the subject with body weight of between 40 and 100 kg every two weeks (Q2W) for three times, and thereafter every four weeks (Q4W).

[C2.82] Satralizumab for use in treating chronic inactive thyroid eye disease (TED) in a subject, wherein the subject has never smoked; and wherein 180 mg of satralizumab for each administration is administered subcutaneously to the subject with body weight of between 40 and 100 kg every two weeks (Q2W) for three times, and thereafter every four weeks (Q4W).

[C2.83] Satralizumab for use in treating chronic inactive thyroid eye disease (TED) in a subject, wherein the subject has never smoked; and wherein 180 mg of satralizumab for each administration is administered subcutaneously to the subject with body weight of over 100 kg every two weeks (Q2W) for three times, and thereafter every four weeks (Q4W).

[C2.84] Satralizumab for use in treating chronic inactive thyroid eye disease (TED) in a subject, wherein the subject has never smoked; and wherein 240 mg of satralizumab for each administration is administered subcutaneously to the subject with body weight of over 100 kg every two weeks (Q2W) for three times, and thereafter every four weeks (Q4W).

[C2.85] Satralizumab for use in preventing thyroid eye disease (TED) in a subject.

[C2.86] Satralizumab for use in preventing thyroid eye disease (TED) in a subject; wherein 60 mg of satralizumab for each administration is administered subcutaneously to the subject with body weight of less than 40 kg every two weeks (Q2W) for three times, and thereafter every four weeks (Q4W).

[C2.87] Satralizumab for use in preventing thyroid eye disease (TED) in a subject; wherein 120 mg of satralizumab for each administration is administered subcutaneously to the subject with body weight of less than 40 kg every two weeks (Q2W) for three times, and thereafter every four weeks (Q4W).

[C2.88] Satralizumab for use in preventing thyroid eye disease (TED) in a subject; wherein 120 mg of satralizumab for each administration is administered subcutaneously to the subject with body weight of between 40 and 100 kg every two weeks (Q2W) for three times, and thereafter every four weeks (Q4W).

[C2.89] Satralizumab for use in preventing thyroid eye disease (TED) in a subject; wherein 180 mg of satralizumab for each administration is administered subcutaneously to the subject with body weight of between 40 and 100 kg every two weeks (Q2W) for three times, and thereafter every four weeks (Q4W).

[C2.90] Satralizumab for use in preventing thyroid eye disease (TED) in a subject; wherein 180 mg of satralizumab for each administration is administered subcutaneously to the subject with body weight of over 100 kg every two weeks (Q2W) for three times, and thereafter every four weeks (Q4W).

[C2.91] Satralizumab for use in preventing thyroid eye disease (TED) in a subject; wherein 240 mg of satralizumab for each administration is administered subcutaneously to the subject with body weight of over 100 kg every two weeks (Q2W) for three times, and thereafter every four weeks (Q4W).

[C2.92] Satralizumab for use in preventing moderate-to-severe active thyroid eye disease (TED) in a subject.

[C2.93] Satralizumab for use in preventing moderate-to-severe active thyroid eye disease (TED) in a subject; wherein 60 mg of satralizumab for each administration is administered subcutaneously to the subject with body weight of less than 40 kg every two weeks (Q2W) for three times, and thereafter every four weeks (Q4W).

[C2.94] Satralizumab for use in preventing moderate-to-severe active thyroid eye disease (TED) in a subject; wherein 120 mg of satralizumab for each administration is administered subcutaneously to the subject with body weight of less than 40 kg every two weeks (Q2W) for three times, and thereafter every four weeks (Q4W).

[C2.95] Satralizumab for use in preventing moderate-to-severe active thyroid eye disease (TED) in a subject; wherein 120 mg of satralizumab for each administration is administered subcutaneously to the subject with body weight of between 40 and 100 kg every two weeks (Q2W) for three times, and thereafter every four weeks (Q4W).

[C2.96] Satralizumab for use in preventing moderate-to-severe active thyroid eye disease (TED) in a subject; wherein 180 mg of satralizumab for each administration is administered subcutaneously to the subject with body weight of between 40 and 100 kg every two weeks (Q2W) for three times, and thereafter every four weeks (Q4W).

[C2.97] Satralizumab for use in preventing moderate-to-severe active thyroid eye disease (TED) in a subject; wherein 180 mg of satralizumab for each administration is administered subcutaneously to the subject with body weight of over 100 kg every two weeks (Q2W) for three times, and thereafter every four weeks (Q4W).

[C2.98] Satralizumab for use in preventing moderate-to-severe active thyroid eye disease (TED) in a subject; wherein 240 mg of satralizumab for each administration is administered subcutaneously to the subject with body weight of over 100 kg every two weeks (Q2W) for three times, and thereafter every four weeks (Q4W).

[C2.99] Satralizumab for use in preventing chronic inactive thyroid eye disease (TED) in a subject.

[C2.100] Satralizumab for use in preventing chronic inactive thyroid eye disease (TED) in a subject; wherein 60 mg of satralizumab for each administration is administered subcutaneously to the subject with body weight of less than 40 kg every two weeks (Q2W) for three times, and thereafter every four weeks (Q4W).

[C2.101] Satralizumab for use in preventing chronic inactive thyroid eye disease (TED) in a subject; wherein 120 mg of satralizumab for each administration is administered subcutaneously to the subject with body weight of less than 40 kg every two weeks (Q2W) for three times, and thereafter every four weeks (Q4W).

[C2.102] Satralizumab for use in preventing chronic inactive thyroid eye disease (TED) in a subject; wherein 120 mg of satralizumab for each administration is administered subcutaneously to the subject with body weight of between 40 and 100 kg every two weeks (Q2W) for three times, and thereafter every four weeks (Q4W).

[C2.103] Satralizumab for use in preventing chronic inactive thyroid eye disease (TED) in a subject; wherein 180 mg of satralizumab for each administration is administered subcutaneously to the subject with body weight of between 40 and 100 kg every two weeks (Q2W) for three times, and thereafter every four weeks (Q4W).

[C2.104] Satralizumab for use in preventing chronic inactive thyroid eye disease (TED) in a subject; wherein 180 mg of satralizumab for each administration is administered subcutaneously to the subject with body weight of over 100 kg every two weeks (Q2W) for three times, and thereafter every four weeks (Q4W).

[C2.105] Satralizumab for use in preventing chronic inactive thyroid eye disease (TED) in a subject; wherein 240 mg of satralizumab for each administration is administered subcutaneously to the subject with body weight of over 100 kg every two weeks (Q2W) for three times, and thereafter every four weeks (Q4W).

[C2.106] Satralizumab for use in preventing thyroid eye disease (TED) in a subject, wherein the subject is a current smoker.

[C2.107] Satralizumab for use in preventing thyroid eye disease (TED) in a subject, wherein the subject is a current smoker; and wherein 60 mg of satralizumab for each administration is administered subcutaneously to the subject with body weight of less than 40 kg every two weeks (Q2W) for three times, and thereafter every four weeks (Q4W).

[C2.108] Satralizumab for use in preventing thyroid eye disease (TED) in a subject, wherein the subject is a current smoker; and wherein 120 mg of satralizumab for each administration is administered subcutaneously to the subject with body weight of less than 40 kg every two weeks (Q2W) for three times, and thereafter every four weeks (Q4W).

[C2.109] Satralizumab for use in preventing thyroid eye disease (TED) in a subject, wherein the subject is a current smoker; and wherein 120 mg of satralizumab for each administration is administered subcutaneously to the subject with body weight of between 40 and 100 kg every two weeks (Q2W) for three times, and thereafter every four weeks (Q4W).

[C2.110] Satralizumab for use in preventing thyroid eye disease (TED) in a subject, wherein the subject is a current smoker; and wherein 180 mg of satralizumab for each administration is administered subcutaneously to the subject with body weight of between 40 and 100 kg every two weeks (Q2W) for three times, and thereafter every four weeks (Q4W).

[C2.111] Satralizumab for use in preventing thyroid eye disease (TED) in a subject, wherein the subject is a current smoker; and wherein 180 mg of satralizumab for each administration is administered subcutaneously to the subject with body weight of over 100 kg every two weeks (Q2W) for three times, and thereafter every four weeks (Q4W).

[C2.112] Satralizumab for use in preventing thyroid eye disease (TED) in a subject, wherein the subject is a current smoker; and wherein 240 mg of satralizumab for each administration is administered subcutaneously to the subject with body weight of over 100 kg every two weeks (Q2W) for three times, and thereafter every four weeks (Q4W).

[C2.113] Satralizumab for use in preventing moderate-to-severe active thyroid eye disease (TED) in a subject, wherein the subject is a current smoker.

[C2.114] Satralizumab for use in preventing moderate-to-severe active thyroid eye disease (TED) in a subject, wherein the subject is a current smoker; and wherein 60 mg of satralizumab for each administration is administered subcutaneously to the subject with body weight of less than 40 kg every two weeks (Q2W) for three times, and thereafter every four weeks (Q4W).

[C2.115] Satralizumab for use in preventing moderate-to-severe active thyroid eye disease (TED) in a subject, wherein the subject is a current smoker; and wherein 120 mg of satralizumab for each administration is administered subcutaneously to the subject with body weight of less than 40 kg every two weeks (Q2W) for three times, and thereafter every four weeks (Q4W).

[C2.116] Satralizumab for use in preventing moderate-to-severe active thyroid eye disease (TED) in a subject, wherein the subject is a current smoker; and wherein 120 mg of satralizumab for each administration is administered subcutaneously to the subject with body weight of between 40 and 100 kg every two weeks (Q2W) for three times, and thereafter every four weeks (Q4W).

[C2.117] Satralizumab for use in preventing moderate-to-severe active thyroid eye disease (TED) in a subject, wherein the subject is a current smoker; and wherein 180 mg of satralizumab for each administration is administered subcutaneously to the subject with body weight of between 40 and 100 kg every two weeks (Q2W) for three times, and thereafter every four weeks (Q4W).

[C2.118] Satralizumab for use in preventing moderate-to-severe active thyroid eye disease (TED) in a subject, wherein the subject is a current smoker; and wherein 180 mg of satralizumab for each administration is administered subcutaneously to the subject with body weight of over 100 kg every two weeks (Q2W) for three times, and thereafter every four weeks (Q4W).

[C2.119] Satralizumab for use in preventing moderate-to-severe active thyroid eye disease (TED) in a subject, wherein the subject is a current smoker; and wherein 240 mg of satralizumab for each administration is administered subcutaneously to the subject with body weight of over 100 kg every two weeks (Q2W) for three times, and thereafter every four weeks (Q4W).

[C2.120] Satralizumab for use in preventing chronic inactive thyroid eye disease (TED) in a subject, wherein the subject is a current smoker.

[C2.121] Satralizumab for use in preventing chronic inactive thyroid eye disease (TED) in a subject, wherein the subject is a current smoker; and wherein 60 mg of satralizumab for each administration is administered subcutaneously to the subject with body weight of less than 40 kg every two weeks (Q2W) for three times, and thereafter every four weeks (Q4W).

[C2.122] Satralizumab for use in preventing chronic inactive thyroid eye disease (TED) in a subject, wherein the subject is a current smoker; and wherein 120 mg of satralizumab for each administration is administered subcutaneously to the subject with body weight of less than 40 kg every two weeks (Q2W) for three times, and thereafter every four weeks (Q4W).

[C2.123] Satralizumab for use in preventing chronic inactive thyroid eye disease (TED) in a subject, wherein the subject is a current smoker; and wherein 120 mg of satralizumab for each administration is administered subcutaneously to the subject with body weight of between 40 and 100 kg every two weeks (Q2W) for three times, and thereafter every four weeks (Q4W).

[C2.124] Satralizumab for use in preventing chronic inactive thyroid eye disease (TED) in a subject, wherein the subject is a current smoker; and wherein 180 mg of satralizumab for each administration is administered subcutaneously to the subject with body weight of between 40 and 100 kg every two weeks (Q2W) for three times, and thereafter every four weeks (Q4W).

[C2.125] Satralizumab for use in preventing chronic inactive thyroid eye disease (TED) in a subject, wherein the subject is a current smoker; and wherein 180 mg of satralizumab for each administration is administered subcutaneously to the subject with body weight of over 100 kg every two weeks (Q2W) for three times, and thereafter every four weeks (Q4W).

[C2.126] Satralizumab for use in preventing chronic inactive thyroid eye disease (TED) in a subject, wherein the subject is a current smoker; and wherein 240 mg of satralizumab for each administration is administered subcutaneously to the subject with body weight of over 100 kg every two weeks (Q2W) for three times, and thereafter every four weeks (Q4W).

[C2.127] Satralizumab for use in preventing thyroid eye disease (TED) in a subject, wherein the subject is a former smoker.

[C2.128] Satralizumab for use in preventing thyroid eye disease (TED) in a subject, wherein the subject is a former smoker; and wherein 60 mg of satralizumab for each administration is administered subcutaneously to the subject with body weight of less than 40 kg every two weeks (Q2W) for three times, and thereafter every four weeks (Q4W).

[C2.129] Satralizumab for use in preventing thyroid eye disease (TED) in a subject, wherein the subject is a former smoker; and wherein 120 mg of satralizumab for each administration is administered subcutaneously to the subject with body weight of less than 40 kg every two weeks (Q2W) for three times, and thereafter every four weeks (Q4W).

[C2.130] Satralizumab for use in preventing thyroid eye disease (TED) in a subject, wherein the subject is a former smoker; and wherein 120 mg of satralizumab for each administration is administered subcutaneously to the subject with body weight of between 40 and 100 kg every two weeks (Q2W) for three times, and thereafter every four weeks (Q4W).

[C2.131] Satralizumab for use in preventing thyroid eye disease (TED) in a subject, wherein the subject is a former smoker; and wherein 180 mg of satralizumab for each administration is administered subcutaneously to the subject with body weight of between 40 and 100 kg every two weeks (Q2W) for three times, and thereafter every four weeks (Q4W).

[C2.132] Satralizumab for use in preventing thyroid eye disease (TED) in a subject, wherein the subject is a former smoker; and wherein 180 mg of satralizumab for each administration is administered subcutaneously to the subject with body weight of over 100 kg every two weeks (Q2W) for three times, and thereafter every four weeks (Q4W).

[C2.133] Satralizumab for use in preventing thyroid eye disease (TED) in a subject, wherein the subject is a former smoker; and wherein 240 mg of satralizumab for each administration is administered subcutaneously to the subject with body weight of over 100 kg every two weeks (Q2W) for three times, and thereafter every four weeks (Q4W).

[C2.134] Satralizumab for use in preventing moderate-to-severe active thyroid eye disease (TED) in a subject, wherein the subject is a former smoker.

[C2.135] Satralizumab for use in preventing moderate-to-severe active thyroid eye disease (TED) in a subject, wherein the subject is a former smoker; and wherein 60 mg of satralizumab for each administration is administered subcutaneously to the subject with body weight of less than 40 kg every two weeks (Q2W) for three times, and thereafter every four weeks (Q4W).

[C2.136] Satralizumab for use in preventing moderate-to-severe active thyroid eye disease (TED) in a subject, wherein the subject is a former smoker; and wherein 120 mg of satralizumab for each administration is administered subcutaneously to the subject with body weight of less than 40 kg every two weeks (Q2W) for three times, and thereafter every four weeks (Q4W).

[C2.137] Satralizumab for use in preventing moderate-to-severe active thyroid eye disease (TED) in a subject, wherein the subject is a former smoker; and wherein 120 mg of satralizumab for each administration is administered subcutaneously to the subject with body weight of between 40 and 100 kg every two weeks (Q2W) for three times, and thereafter every four weeks (Q4W).

[C2.138] Satralizumab for use in preventing moderate-to-severe active thyroid eye disease (TED) in a subject, wherein the subject is a former smoker; and wherein 180 mg of satralizumab for each administration is administered subcutaneously to the subject with body weight of between 40 and 100 kg every two weeks (Q2W) for three times, and thereafter every four weeks (Q4W).

[C2.139] Satralizumab for use in preventing moderate-to-severe active thyroid eye disease (TED) in a subject, wherein the subject is a former smoker; and wherein 180 mg of satralizumab for each administration is administered subcutaneously to the subject with body weight of over 100 kg every two weeks (Q2W) for three times, and thereafter every four weeks (Q4W).

[C2.140] Satralizumab for use in preventing moderate-to-severe active thyroid eye disease (TED) in a subject, wherein the subject is a former smoker; and wherein 240 mg of satralizumab for each administration is administered subcutaneously to the subject with body weight of over 100 kg every two weeks (Q2W) for three times, and thereafter every four weeks (Q4W).

[C2.141] Satralizumab for use in preventing chronic inactive thyroid eye disease (TED) in a subject, wherein the subject is a former smoker.

[C2.142] Satralizumab for use in preventing chronic inactive thyroid eye disease (TED) in a subject, wherein the subject is a former smoker; and wherein 60 mg of satralizumab for each administration is administered subcutaneously to the subject with body weight of less than 40 kg every two weeks (Q2W) for three times, and thereafter every four weeks (Q4W).

[C2.143] Satralizumab for use in preventing chronic inactive thyroid eye disease (TED) in a subject, wherein the subject is a former smoker; and wherein 120 mg of satralizumab for each administration is administered subcutaneously to the subject with body weight of less than 40 kg every two weeks (Q2W) for three times, and thereafter every four weeks (Q4W).

[C2.144] Satralizumab for use in preventing chronic inactive thyroid eye disease (TED) in a subject, wherein the subject is a former smoker; and wherein 120 mg of satralizumab for each administration is administered subcutaneously to the subject with body weight of between 40 and 100 kg every two weeks (Q2W) for three times, and thereafter every four weeks (Q4W).

[C2.145] Satralizumab for use in preventing chronic inactive thyroid eye disease (TED) in a subject, wherein the subject is a former smoker; and wherein 180 mg of satralizumab for each administration is administered subcutaneously to the subject with body weight of between 40 and 100 kg every two weeks (Q2W) for three times, and thereafter every four weeks (Q4W).

[C2.146] Satralizumab for use in preventing chronic inactive thyroid eye disease (TED) in a subject, wherein the subject is a former smoker; and wherein 180 mg of satralizumab for each administration is administered subcutaneously to the subject with body weight of over 100 kg every two weeks (Q2W) for three times, and thereafter every four weeks (Q4W).

[C2.147] Satralizumab for use in preventing chronic inactive thyroid eye disease (TED) in a subject, wherein the subject is a former smoker; and wherein 240 mg of satralizumab for each administration is administered subcutaneously to the subject with body weight of over 100 kg every two weeks (Q2W) for three times, and thereafter every four weeks (Q4W).

[C2.148] Satralizumab for use in preventing thyroid eye disease (TED) in a subject, wherein the subject has never smoked.

[C2.149] Satralizumab for use in preventing thyroid eye disease (TED) in a subject, wherein the subject has never smoked; and wherein 60 mg of satralizumab for each administration is administered subcutaneously to the subject with body weight of less than 40 kg every two weeks (Q2W) for three times, and thereafter every four weeks (Q4W).

[C2.150] Satralizumab for use in preventing thyroid eye disease (TED) in a subject, wherein the subject has never smoked; and wherein 120 mg of satralizumab for each administration is administered subcutaneously to the subject with body weight of less than 40 kg every two weeks (Q2W) for three times, and thereafter every four weeks (Q4W).

[C2.151] Satralizumab for use in preventing thyroid eye disease (TED) in a subject, wherein the subject has never smoked; and wherein 120 mg of satralizumab for each administration is administered subcutaneously to the subject with body weight of between 40 and 100 kg every two weeks (Q2W) for three times, and thereafter every four weeks (Q4W).

[C2.152] Satralizumab for use in preventing thyroid eye disease (TED) in a subject, wherein the subject has never smoked; and wherein 180 mg of satralizumab for each administration is administered subcutaneously to the subject with body weight of between 40 and 100 kg every two weeks (Q2W) for three times, and thereafter every four weeks (Q4W).

[C2.153] Satralizumab for use in preventing thyroid eye disease (TED) in a subject, wherein the subject has never smoked; and wherein 180 mg of satralizumab for each administration is administered subcutaneously to the subject with body weight of over 100 kg every two weeks (Q2W) for three times, and thereafter every four weeks (Q4W).

[C2.154] Satralizumab for use in preventing thyroid eye disease (TED) in a subject, wherein the subject has never smoked; and wherein 240 mg of satralizumab for each administration is administered subcutaneously to the subject with body weight of over 100 kg every two weeks (Q2W) for three times, and thereafter every four weeks (Q4W).

[C2.155] Satralizumab for use in preventing moderate-to-severe active thyroid eye disease (TED) in a subject, wherein the subject has never smoked.

[C2.156] Satralizumab for use in preventing moderate-to-severe active thyroid eye disease (TED) in a subject, wherein the subject has never smoked; and wherein 60 mg of satralizumab for each administration is administered subcutaneously to the subject with body weight of less than 40 kg every two weeks (Q2W) for three times, and thereafter every four weeks (Q4W).

[C2.157] Satralizumab for use in preventing moderate-to-severe active thyroid eye disease (TED) in a subject, wherein the subject has never smoked; and wherein 120 mg of satralizumab for each administration is administered subcutaneously to the subject with body weight of less than 40 kg every two weeks (Q2W) for three times, and thereafter every four weeks (Q4W).

[C2.158] Satralizumab for use in preventing moderate-to-severe active thyroid eye disease (TED) in a subject, wherein the subject has never smoked; and wherein 120 mg of satralizumab for each administration is administered subcutaneously to the subject with body weight of between 40 and 100 kg every two weeks (Q2W) for three times, and thereafter every four weeks (Q4W).

[C2.159] Satralizumab for use in preventing moderate-to-severe active thyroid eye disease (TED) in a subject, wherein the subject has never smoked; and wherein 180 mg of satralizumab for each administration is administered subcutaneously to the subject with body weight of between 40 and 100 kg every two weeks (Q2W) for three times, and thereafter every four weeks (Q4W).

[C2.160] Satralizumab for use in preventing moderate-to-severe active thyroid eye disease (TED) in a subject, wherein the subject has never smoked; and wherein 180 mg of satralizumab for each administration is administered subcutaneously to the subject with body weight of over 100 kg every two weeks (Q2W) for three times, and thereafter every four weeks (Q4W).

[C2.161] Satralizumab for use in preventing moderate-to-severe active thyroid eye disease (TED) in a subject, wherein the subject has never smoked; and wherein 240 mg of satralizumab for each administration is administered subcutaneously to the subject with body weight of over 100 kg every two weeks (Q2W) for three times, and thereafter every four weeks (Q4W).

[C2.162] Satralizumab for use in preventing chronic inactive thyroid eye disease (TED) in a subject, wherein the subject has never smoked.

[C2.163] Satralizumab for use in preventing chronic inactive thyroid eye disease (TED) in a subject, wherein the subject has never smoked; and wherein 60 mg of satralizumab for each administration is administered subcutaneously to the subject with body weight of less than 40 kg every two weeks (Q2W) for three times, and thereafter every four weeks (Q4W).

[C2.164] Satralizumab for use in preventing chronic inactive thyroid eye disease (TED) in a subject, wherein the subject has never smoked; and wherein 120 mg of satralizumab for each administration is administered subcutaneously to the subject with body weight of less than 40 kg every two weeks (Q2W) for three times, and thereafter every four weeks (Q4W).

[C2.165] Satralizumab for use in preventing chronic inactive thyroid eye disease (TED) in a subject, wherein the subject has never smoked; and wherein 120 mg of satralizumab for each administration is administered subcutaneously to the subject with body weight of between 40 and 100 kg every two weeks (Q2W) for three times, and thereafter every four weeks (Q4W).

[C2.166] Satralizumab for use in preventing chronic inactive thyroid eye disease (TED) in a subject, wherein the subject has never smoked; and wherein 180 mg of satralizumab for each administration is administered subcutaneously to the subject with body weight of between 40 and 100 kg every two weeks (Q2W) for three times, and thereafter every four weeks (Q4W).

[C2.167] Satralizumab for use in preventing chronic inactive thyroid eye disease (TED) in a subject, wherein the subject has never smoked; and wherein 180 mg of satralizumab for each administration is administered subcutaneously to the subject with body weight of over 100 kg every two weeks (Q2W) for three times, and thereafter every four weeks (Q4W).

[C2.168] Satralizumab for use in preventing chronic inactive thyroid eye disease (TED) in a subject, wherein the subject has never smoked; and wherein 240 mg of satralizumab for each administration is administered subcutaneously to the subject with body weight of over 100 kg every two weeks (Q2W) for three times, and thereafter every four weeks (Q4W).

[D1] A kit for treating or preventing thyroid eye disease (TED) in a subject, comprising:
(1) the pharmaceutical composition of any one of A2.1-A2.29; and
(2) a package insert or label instructing administration of the pharmaceutical composition to a subject.

[E1] A method of treating thyroid eye disease (TED) in a subject, the method comprising administering to the subject an effective amount of an IL-6 inhibitor.

[E2] The method of E1, wherein the IL-6 inhibitor is an anti-IL-6 antibody or antigen-binding fragment thereof, or an anti-IL-6 receptor antibody or antigen binding fragment thereof.

[E3] The method of E1 or E2, wherein the IL-6 inhibitor is an anti-IL-6 receptor antibody or antigen binding fragment thereof.

[E4] The method of any one of E1-E3, wherein the IL-6 inhibitor is a humanized antibody.

[E5] The method of any one of E1-E4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.

[E6] The method of E5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.

[E7] The method of E5 or E6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.

[E8] The method of any one of E5-E7, wherein the IL-6 inhibitor is satralizumab.

[E9] The method of any one of E1-E8, wherein the subject has moderate-to-severe TED.

[E10] The method of any one of E1-E9, wherein the subject has active TED.

[E11] The method of any one of E1-E9, wherein the subject has chronic inactive TED.

[E12] The method of any one of E1-E 11, wherein the subject is a current smoker.

[E13] The method of any one of E1-E 11, wherein the subject is a former smoker.

[E14] The method of any one of E1-E 11, wherein the subject has never smoked.

[E15] The method of any one of E1-E14, wherein the subject is positive for thyrotropin receptor (TSH receptor; TSHR) autoantibodies or insulin-like growth factor-1 receptor (IGF-1 receptor; IGF-1R) autoantibodies.

[E16] The method of any one of E1-E15, wherein the subject has experienced disease progression or relapse of the disease after the steroid treatment.

[E17] The method of any one of E1-E16, wherein the IL-6 inhibitor is administered to the subject subcutaneously.

[E18] The method of any one of E5-E17, wherein the subject is determined to have a body weight of less than 40 kg, and the amount of the anti-IL-6 receptor antibody or antigen binding fragment thereof administered to the subject in each administration is 60 mg or 120 mg; or the subject is determined to have a body weight of between 40 and 100 kg, and the amount of the anti-IL-6 receptor antibody or antigen binding fragment thereof administered to the subject in each administration is 120 mg or 180 mg; or the subject is determined to have a body weight of over 100 kg, and the amount of the anti-IL-6 receptor antibody or antigen binding fragment thereof administered to the subject in each administration is 180 mg or 240 mg.

[E19] The method of any one of E5-E17, wherein the subject is determined to have a body weight of less than 40 kg, and the amount of the anti-IL-6 receptor antibody or antigen binding fragment thereof administered to the subject in each administration is 60 mg.

[E20] The method of any one of E5-E17, wherein the subject is determined to have a body weight of less than 40 kg, and the amount of the anti-IL-6 receptor antibody or antigen binding fragment thereof administered to the subject in each administration is 120 mg.

[E21] The method of any one of E5-E17, wherein the subject is determined to have a body weight of between 40 and 100 kg, and the amount of the anti-IL-6 receptor antibody or antigen binding fragment thereof administered to the subject in each administration is 120 mg.

[E22] The method of any one of E5-E17, wherein the subject is determined to have a body weight of between 40 and 100 kg, and the amount of the anti-IL-6 receptor antibody or antigen binding fragment thereof administered to the subject in each administration is 180 mg.

[E23] The method of any one of E5-E17, wherein the subject is determined to have a body weight of over 100 kg, and the amount of the anti-IL-6 receptor antibody or antigen binding fragment thereof administered to the subject in each administration is 180 mg.

[E24] The method of any one of E5-E17, wherein the subject is determined to have a body weight of over 100 kg, and the amount of the anti-IL-6 receptor antibody or antigen binding fragment thereof administered to the subject in each administration is 240 mg.

[E25] The method of any one of E5-E24, wherein the anti-IL-6 receptor antibody or antigen binding fragment thereof is administered to the subject two weeks after the anti-IL-6 receptor antibody or antigen binding fragment thereof is previously administered to the subject.

[E26] The method of any one of E5-E24, wherein the anti-IL-6 receptor antibody or antigen binding fragment thereof is administered to the subject four weeks after the anti-IL-6 receptor antibody or antigen binding fragment thereof is previously administered to the subject.

[E27] The method of any one of E5-E26, wherein the anti-IL-6 receptor antibody or antigen binding fragment thereof is administered to the subject every two weeks (Q2W) for three times, and thereafter every four weeks (Q4W).

[F28] The method of any one of F5-F27, wherein the anti-IL-6 receptor antibody or antigen binding fragment thereof is administered to the subject at least seven times.

[E29] The method of any one of E1-E28, wherein administering the IL-6 inhibitor to the subject
reduces or improves proptosis;
reduces or improves diplopia;
reduces or improves the CAS score;
improves extraocular motility;
reduces or improves motility-induced orbital pain;
reduces or improves spontaneous orbital pain;
improves the Visual Functioning and Appearance sub-scale scores of the GO-QoL;
improves the OSDI overall scores;
improves the OSDI Ocular Symptoms;
improves the OSDI Vision-related Function subscale scores; and/or
improves Oxford corneal staining scores
   in the subject.

[E30] The method of any one of E1-E29, wherein administering the IL-6 inhibitor to the subject results in one or more of the followings in the subject:
2-mm or more reduction or improvement in proptosis;
1-grade or more reduction or improvement in diplopia;
2-point or more reduction or improvement in the CAS score;
an improvement in extraocular motility;
a reduction or improvement in motility-induced orbital pain;
a reduction or improvement in spontaneous orbital pain;
6-point or more improvement in the Visual Functioning and Appearance sub-scale scores of the GO-QoL;
10-point or more improvement in the OSDI overall scores;
an improvement in the OSDI Ocular Symptoms;
an improvement in the OSDI Vision-related Function subscale scores; or
an improvement in Oxford corneal staining scores.

[F1] A method of preventing thyroid eye disease (TED) in a subject, the method comprising administering to the subject an effective amount of an IL-6 inhibitor.

[F2] The method of F1, wherein the IL-6 inhibitor is an anti-IL-6 antibody or antigen-binding fragment thereof, or an anti-IL-6 receptor antibody or antigen binding fragment thereof.

[F3] The method of F1 or F2, wherein the IL-6 inhibitor is an anti-IL-6 receptor antibody or antigen binding fragment thereof.

[F4] The method of any one of F1-F3, wherein the IL-6 inhibitor is a humanized antibody.

[F5] The method of any one of F1-F4, wherein the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10.

[F6] The method of F5, wherein the anti-IL-6 receptor antibody or antigen binding fragment thereof comprises a VH comprising the amino acid sequence of SEQ ID NO: 1 and a VL comprising the amino acid sequence of SEQ ID NO: 2.

[F7] The method of F5 or F6, wherein the IL-6 inhibitor is an anti-IL-6 receptor antibody comprising a heavy chain comprising the amino acid sequence of SEQ ID NO: 3 and a light chain comprising the amino acid sequence of SEQ ID NO: 4.

[F8] The method of any one of F5-F7, wherein the IL-6 inhibitor is satralizumab.

[F9] The method of any one of F1-F8, wherein preventing TED comprises reducing a risk of relapse of, delaying relapse of, reducing a frequency of relapse of, reducing severity of relapse of, or reducing the need for rescue therapy for relapse of TED in the subject.

[F10] The method of any one of F1-F9, wherein the subject has moderate-to-severe TED.

[F11] The method of any one of F1-F10, wherein the subject has active TED.

[F12] The method of any one of F1-F10, wherein the subject has chronic inactive TED.

[F13] The method of any one of F1-F12, wherein the subject is a current smoker.

[F14] The method of any one of F1-F12, wherein the subject is a former smoker.

[F15] The method of any one of F1-F12, wherein the subject has never smoked.

[F16] The method of any one of F1-F15, wherein the subject is positive for thyrotropin receptor (TSH receptor; TSHR) autoantibodies or insulin-like growth factor-1 receptor (IGF-1 receptor; IGF-1R) autoantibodies.

[F17] The method of any one of F1-F16, wherein the subject has experienced disease progression or relapse of the disease after the steroid treatment.

[F18] The method of any one of F1-F17, wherein the IL-6 inhibitor is administered to the subject subcutaneously.

[F19] The method of any one of F5-F18, wherein the subject is determined to have a body weight of less than 40 kg, and the amount of the anti-IL-6 receptor antibody or antigen binding fragment thereof administered to the subject in each administration is 60 mg or 120 mg; or the subject is determined to have a body weight of between 40 and 100 kg, and the amount of the anti-IL-6 receptor antibody or antigen binding fragment thereof administered to the subject in each administration is 120 mg or 180 mg; or the subject is determined to have a body weight of over 100 kg, and the amount of the anti-IL-6 receptor antibody or antigen binding fragment thereof administered to the subject in each administration is 180 mg or 240 mg.

[F20] The method of any one of F5-F18, wherein the subject is determined to have a body weight of less than 40 kg, and the amount of the anti-IL-6 receptor antibody or antigen binding fragment thereof administered to the subject in each administration is 60 mg.

[F21] The method of any one of F5-F18, wherein the subject is determined to have a body weight of less than 40 kg, and the amount of the anti-IL-6 receptor antibody or antigen binding fragment thereof administered to the subject in each administration is 120 mg.

[F22] The method of any one of F5-F18, wherein the subject is determined to have a body weight of between 40 and 100 kg, and the amount of the anti-IL-6 receptor antibody or antigen binding fragment thereof administered to the subject in each administration is 120 mg.

[F23] The method of any one of F5-F18, wherein the subject is determined to have a body weight of between 40 and 100 kg, and the amount of the anti-IL-6 receptor antibody or antigen binding fragment thereof administered to the subject in each administration is 180 mg.

[F24] The method of any one of F5-F18, wherein the subject is determined to have a body weight of over 100 kg, and the amount of the anti-IL-6 receptor antibody or antigen binding fragment thereof administered to the subject in each administration is 180 mg.

[F25] The method of any one of F5-F18, wherein the subject is determined to have a body weight of over 100 kg, and the amount of the anti-IL-6 receptor antibody or antigen binding fragment thereof administered to the subject in each administration is 240 mg.

[F26] The method of any one of F5-F25, wherein the anti-IL-6 receptor antibody or antigen binding fragment thereof is administered to the subject two weeks after the anti-IL-6 receptor antibody or antigen binding fragment thereof is previously administered to the subject.

[F27] The method of any one of F5-F25, wherein the anti-IL-6 receptor antibody or antigen binding fragment thereof is administered to the subject four weeks after the anti-IL-6 receptor antibody or antigen binding fragment thereof is previously administered to the subject.

[F28] The method of any one of F5-F27, wherein the anti-IL-6 receptor antibody or antigen binding fragment thereof is administered to the subject every two weeks (Q2W) for three times, and thereafter every four weeks (Q4W).

[F29] The method of any one of F5-F28, wherein the anti-IL-6 receptor antibody or antigen binding fragment thereof is administered to the subject at least seven times.

[F30] The method of any one of F1-F29, wherein administering the IL-6 inhibitor to the subject
reduces or improves proptosis;
reduces or improves diplopia;
reduces or improves the CAS score;
improves extraocular motility;
reduces or improves motility-induced orbital pain;
reduces or improves spontaneous orbital pain;
improves the Visual Functioning and Appearance sub-scale scores of the GO-QoL;
improves the OSDI overall scores;
improves the OSDI Ocular Symptoms;
improves the OSDI Vision-related Function subscale scores; and/or
improves Oxford corneal staining scores in the subject.

[F31] The method of any one of F1-F30, wherein administering the IL-6 inhibitor to the subject results in one or more of the followings in the subject:
2-mm or more reduction or improvement in proptosis;
1-grade or more reduction or improvement in diplopia;
2-point or more reduction or improvement in the CAS score;
an improvement in extraocular motility;
a reduction or improvement in motility-induced orbital pain;
a reduction or improvement in spontaneous orbital pain;
6-point or more improvement in the Visual Functioning and Appearance sub-scale scores of the GO-QoL;
10-point or more improvement in the OSDI overall scores;
an improvement in the OSDI Ocular Symptoms;
an improvement in the OSDI Vision-related Function subscale scores; or
an improvement in Oxford corneal staining scores.

### Advantageous Effects of Invention

The present disclosure can provide a medicament (a pharmaceutical composition) comprising satralizumab for treating or preventing TED.

### BRIEF DESCRIPTION OF DRAWINGS

FIG. 1 shows the study schema of Phase III, randomized, double-masked, placebo-controlled, multicenter study to evaluate the efficacy, safety, pharmacokinetics, and pharmacodynamics of subcutaneous satralizumab in patients with moderate-to-severe thyroid eye disease (TED). BL=baseline; Q4W=every 4 weeks; TED=thyroid eye disease. (a) Patients with chronic inactive TED will be capped at 25% of enrollment. (b) Participants randomized 1:1 to receive either satralizumab (body weight-based tiered dosing) or placebo. Dosing on Day 1 and at Weeks 2 and 4, and then at Weeks 8, 12, 16 and 20 (a total of seven doses). (c) Randomization stratified by current smoking status (current smoker, including the use of e-cigarettes vs. former smoker vs. never smoked) and TED disease activity status (active vs. inactive). (d) Participants who received placebo during Part I and commence satralizumab in Part II will receive a loading dose of satralizumab at Week 26; participants who received satralizumab in Part I and continue to receive satralizumab in Part II do not require a loading dose and will receive placebo at Week 26. (e) Rescue intervention is permitted starting from Week 4 based on protocol-defined criteria as assessed by the investigator. After receiving rescue medication, participants will be discontinued from study drug, but will be encouraged to continue in the study and complete all remaining study visit assessments. (f) Based on proptosis response at Week 24 (i.e. the primary endpoint), non-responders will receive satralizumab from Week 24 to Week 44 followed by the final in-person evaluation at Week 48; and responders will be re-randomized to receive placebo or satralizumab with doses starting at Week 24 and continuing through Week 44. After the final non-dosing in-person visit at Week 48, participants will have telephone follow-up every 2 months for 6 months (i.e., at Weeks 56, 64, and 72). (g) Week 24 will be the final visit for Part I and the Day 1 visit for Part II.

FIG. 2 shows the dependencies of steady-state exposure parameters and receptor occupancy on body weight following (i) 60 mg Dosing (30-<40 kg), (ii) 120 mg Q4W (between 40 and 100 kg), and (iii) 180 mg (>100 kg) dosing Assuming Clearance as in NMOSD. ADA=anti-drug antibody; CL=clearance; Cₘₐₓ=maximum concentration observed; C, =steady-state concentration at the end of a dosing interval; NMOSD=neuromyelitis optica spectrum disorder; RO=receptor occupancy; WT=weight. Note: The simulations are based on 2000 individuals. Predictions for Cₘₐₓ am shown on the top, C_{trough} in the middle, and RO on the bottom. Points are simulated data assuming ADA positivity in a similar proportion of participants as was observed in NMOSD studies. Dashed horizontal lines have been added for reference.

### DESCRIPTION OF EMBODIMENTS

In one aspect, the present disclosure relates to a medicament or a pharmaceutical composition for treating or preventing thyroid eye disease (TED) in a subject, comprising an IL-6 inhibitor as an active ingredient. In another aspect, the present disclosure also relates to use of an IL-6 inhibitor in the preparation of a medicament for treating or preventing TED in a subject. In yet another aspect, the present disclosure relates to an IL-6 inhibitor for use in treating or preventing TED in a subject. Furthermore, the present disclosure also relates to a kit for treating or preventing TED in a subject, which comprises a pharmaceutical composition comprising an IL-6 inhibitor, and a package insert or label instructing administration of the pharmaceutical composition to a subject. Moreover, the present disclosure also relates to a method of treating or preventing TED in a subject, the method comprising administering to the subject an effective amount of an IL-6 inhibitor.

An “IL-6 inhibitor” of the present disclosure is a substance that blocks signal transduction by IL-6 and inhibits the biological activities of IL-6. The IL-6 inhibitor is preferably a substance that inhibits binding between IL-6 and IL-6 receptor and/or between the IL-6/IL-6 receptor complex and gp130. Examples of an IL-6 inhibitor of the present disclosure include, but are not particularly limited to, an anti-IL-6 antibody or antigen binding fragment thereof, an anti-IL-6 receptor antibody or antigen binding fragment thereof, an anti-gp130 antibody or antigen binding fragment thereof, an IL-6 variant, a soluble IL-6 receptor variant, or a partial peptide of IL-6 or IL-6 receptor, and a low-molecular-weight substance showing a similar activity. Examples of an IL-6 inhibitor of the present disclosure may be preferably an anti-IL-6 antibody or antigen-binding fragment thereof, or an anti-IL-6 receptor antibody or antigen binding fragment thereof, more preferably an anti-IL-6 receptor antibody or antigen binding fragment thereof, optionally a humanized antibody.

In some embodiments of the present disclosure, the IL-6 inhibitor is an anti-IL-6 receptor antibody or antigen binding fragment thereof comprising a heavy chain variable region (VH) CDR1 comprising the amino acid sequence of SEQ ID NO: 5, a VH CDR2 comprising the amino acid sequence of SEQ ID NO: 6, a VH CDR3 comprising the amino acid sequence of SEQ ID NO: 7, a light chain variable region (VL) CDR1 comprising the amino acid sequence of SEQ ID NO: 8, a VL CDR2 comprising the amino acid sequence of SEQ ID NO: 9, and a VL CDR3 comprising the amino acid sequence of SEQ ID NO: 10. In certain embodiments in the present disclosure, the anti-IL-6 receptor antibody or antigen binding fragment thereof comprises a VH comprising the amino acid sequence of SEQ ID NO: 1 and a VL comprising the amino acid sequence of SEQ ID NO: 2. In certain embodiments in the present disclosure, the IL-6 inhibitor is an anti-IL-6 receptor antibody comprising a heavy chain comprising the amino acid sequence of SEQ ID NO: 3 and a light chain comprising the amino acid sequence of SEQ ID NO: 4. In certain embodiments in the present disclosure, the IL-6 inhibitor is satralizumab. In some embodiments of the present disclosure, the term “satralizumab” is an anti-IL-6 receptor antibody that comprises a heavy-chain CDR1 comprising the amino acid sequence of SEQ ID NO: 5, a heavy-chain CDR2 comprising the amino acid sequence of SEQ ID NO: 6, a heavy-chain CDR3 comprising the amino acid sequence of SEQ ID NO: 7, a light-chain CDR1 comprising the amino acid sequence of SEQ ID NO: 8, a light-chain CDR2 comprising the amino acid sequence of SEQ ID NO: 9, and a light-chain CDR3 comprising the amino acid sequence of SEQ ID NO: 10; preferably comprises a heavy-chain variable region comprising the amino acid sequence of SEQ ID NO: 1 and a light-chain variable region comprising the amino acid sequence of SEQ ID NO: 2; and most preferably comprises a heavy chain comprising the amino acid sequence of SEQ ID NO: 3 and a light chain comprising the amino acid sequence of SEQ ID NO: 4.

Thyroid eye disease (TED) [synonyms: Graves' ophthalmopathy/orbitopathy (GO), thyroid-associated orbitopathy (TAO)] is
a complex orbital inflammatory disease of autoimmune etiology;
common manifestation of Graves' disease (GD); and
sight-threatening and disfiguring.

TED is a disease in which autoantibodies against TSHR (thyroid stimulating hormone receptor), IGF-1R (insulin-like growth factor-1 receptor), etc. triggers inflammation in extraocular muscles and retrobulbar tissues, affecting not only external changes but also visual function. Approx. 25-50% of patients with GD develops TED. No precise epidemiological data of the disease exist in global and also Japan. The estimated number of TED patients in Japan is 32,500-65,000, however, moderate-to-severe TED is believed to affect only a much smaller (approx. 20% of the overall) patient subpopulation. The following symptoms are known to be associated with TED: eyelid retraction, proptosis, periorbital edema and erythema, restrictive strabismus, diplopia, corneal ulceration, optic neuropathy (rare).

### Diagnosis and Assessment of TED

For diagnosis and assessment of TED, cooperation among thyroidologists, ophthalmologists and radiologists is important. Diagnosis of TED requires diagnosis of a thyroid disease (e.g., Graves' disease) and an ophthalmopathy. To assess the severity and activity of ophthalmopathy and to make differential diagnosis, various methods of imaging including MRI and CT of the orbit can be performed. However, imaging is not common in the US and Europe.

### Disease Severity Classification of TED

There are two major similar classifications of TED: (1) the EUGOGO (European Group on GO) classification (Bartalena L, Kahaly G J, et al., European Journal of Endocrinology (2021) 185, G43-G67) and (2) the NOSPECS/Inoue classification (The Japan Thyroid Association and The Japan Endocrine Society, Treatment guide for thyroid-associated ophthalmopathy). In the Japanese guideline, both classifications are quoted.

### Disease Activity Assessment of TED

In the present disclosure, the term “proptosis” refers to the forward projection, displacement, bulging, or protrusion of the eye anteriorly out of the orbit. Owing to the rigid bony structure of the orbit with only anterior opening for expansion, any increase in orbital soft tissue contents taking place from the side or from behind will displace the eyeball forward. Thyroid eye disease (e.g., TED) is currently recognized as the most common cause of proptosis in adults. For example, techniques for measuring and assessing proptosis are known in the art and can be routinely applied in the present disclosure. Measurement of the degree of proptosis can be performed using an exophthalmometer, an instrument used for measuring the degree of forward displacement of the eye. Other techniques including, but not limited to ultrasonography, computerized tomography, and scanning and magnetic resonance imaging (MRI) may also be used in evaluating the degree of proptosis. In some embodiments, the subject administered a treatment provided herein has at 2 mm or more reduction or improvement in proptosis compared to prior to receiving the treatment.

Diplopia, also known as double vision, can be assessed using any technique known in the art. In some embodiments, diplopia is assessed use the Gorman subjective diplopia score (range: 0 to 3 points), which includes: no diplopia (absent, scored as 0), diplopia in the primary position of gaze when the patient is tired or awakening (intermittent, scored as 1), diplopia at extremes of gaze (inconstant, scored as 2), and continuous diplopia in the primary or reading position (constant, scored as 3).

Bartalena et al., Eur. J. Endocrinol. 158:273-85 (2008). A Gorman subjective diplopia score grade of >1 point improvement is considered clinically meaningful. In some embodiments, the subject administered a treatment provided herein has a grade of 1 point or more reduction or improvement in diplopia.

The Clinical Activity Score (CAS) is a validated scoring system for assessing the disease activity of TED based on classical features of inflammation in TED. Each item is represented as 1 point. 4-points or more out of 10-items and 3-points or more out of the first 7-items suggest active TED. In Japanese, MRI may show inflammatory findings even if CAS is scored as 1-2 points. 2 or more points decrease of CAS can be considered as clinically meaningful. In some embodiments, the subject administered a treatment provided herein has at 2-point or more reduction or improvement in CAS score compared to prior to receiving the treatment.

The Ocular Surface Disease Index (OSDI) is a validated 12-item questionnaire that provides a rapid assessment of the symptoms of ocular irritation consistent with ocular surface inflammatory disorders, including TED, and their impact on vision-related functioning. See e.g., Schiffman et al., Arch. Ophthalmol. 118: 615-621 (2000). The OSDI assesses ocular symptoms (the Ocular Symptoms subscale), their impact on patient vision-related functioning (Vision Related Function subscale), and environmental factors triggering the symptoms (Environmental Triggers subscale). The 12 items of the OSDI questionnaire are graded on a scale of 0 to 4. Scores are derived based on responses to provide an OSDI score on a scale of 0 to 100, with higher scores representing greater disability. A negative change from baseline indicates an improvement in vision-related function and the ocular inflammatory disorders. In some embodiments, the subject administered a treatment provided herein has at 10-point or more improvement in OSDI overall score compared to prior to receiving the treatment. The Graves' Ophthalmopathy-specific Quality-of-Life scale (GO-QOL) is designed to determine the improved quality of life after treatment. The GO-QOL has two self-assessment subscales. The first relates to the impact of visual function on daily activities, while the second relates to the impact of self-perceived appearance. Scores on the visual-functioning subscale range from 0 to 100. In some embodiments, a subject administered a treatment provided herein has a 6-point or more improvement in the Visual Functioning and Appearance sub-scale scores of the GO-QoL.

In the present disclosure, “a subject”, which may be referred as “a patient”, has the above-described Thyroid Eye Disease (TED) regardless of Thyroid Eye Disease activity or duration. In one embodiment, the subject has moderate-to-severe TED. In one embodiment, the subject has active TED. In certain embodiments, the subject has 3 or more symptoms on the CAS 7-item scale. In another embodiment, the subject has chronic inactive TED. In certain embodiments, the subject has less than 3 symptoms on the CAS 7-point scale. In one embodiment, the subject is a current smoker. In another embodiment, the subject is a former smoker. In other embodiments, the subject has never smoked. In one embodiment, the subject is positive for thyrotropin receptor (TSH receptor; TSHR) autoantibodies or insulin-like growth factor-1 receptor (IGF-1 receptor; IGF-1R) autoantibodies. In one embodiment, whether or not, the subject is positive for TSHR autoantibodies or IGF-1R autoantibodies is detected in blood samples from the subject. In one embodiment, the subject has experienced disease progression or relapse of the disease after the steroid treatment.

In the present disclosure, the term “treatment” or “treating” is used with the meaning that even if complete remission of TED is not achieved, alleviating or improving symptoms to a level at which minimal manifestations (MM) can be maintained, or maintaining such a state, is also included in the “treatment” of TED.

In the present disclosure, the phrase “preventing TED” comprises reducing a risk of relapse of, delaying relapse of, reducing a frequency of relapse of, reducing severity of relapse of, or reducing the need for rescue therapy for relapse of TED.

In the present disclosure, the efficacy of IL-6 inhibitor such as satralizumab for the treatment of TED may be assessed based on one or more aspects and/or scores as described below.
a reduction or improvement in proptosis;
a reduction or improvement in diplopia;
a reduction or improvement in the CAS score;
an improvement in extraocular motility;
a reduction or improvement in motility-induced orbital pain;
a reduction or improvement in spontaneous orbital pain;
an improvement in the Visual Functioning and Appearance sub-scale scores of the GO-QoL;
an improvement in the OSDI overall scores;
an improvement in the OSDI Ocular Symptoms;
an improvement in the OSDI Vision-related Function subscale scores; or
an improvement in Oxford corneal staining scores.

In certain embodiments, the medicament or the pharmaceutical composition of the present disclosure can result in one or more of the followings in the subject:
2-mm or more reduction or improvement in proptosis;
1-grade or more reduction or improvement in diplopia;
2-point or more reduction or improvement in the CAS score;
an improvement in extraocular motility;
a reduction or improvement in motility-induced orbital pain;
a reduction or improvement in spontaneous orbital pain;
6-point or more improvement in the Visual Functioning and Appearance sub-scale scores of the GO-QoL;
10-point or more improvement in the OSDI overall scores;
an improvement in the OSDI Ocular Symptoms;
an improvement in the OSDI Vision-related Function subscale scores; or
an improvement in Oxford corneal staining scores.

In certain embodiments, the medicament or the pharmaceutical composition of the present disclosure can delay the time from an administration of the IL-6 inhibitor to the first occurrence of a relapse of thyroid eye disease (TED) in a subject including but not limited to anti-TSHR positive subject and anti-IGF-1R positive subject. In certain embodiments, the medicament or the pharmaceutical composition of the present disclosure can further reduce one or more of the followings in the subject:
the rate of relapses of TED;
the proportion of subjects requiring surgical intervention for TED;
the proportion of subjects receiving rescue therapy for TED; or
the rate of inpatient hospitalizations.

The given period of applying the present disclosure (e.g., administering a medicament or a pharmaceutical composition of the present disclosure) for evaluating efficacy is not particularly limited and includes 1 week, 2 weeks, 4 weeks, 8 weeks, 12 weeks, 24 weeks, 48 weeks, 1 year, 2 years, 3 years, 4 years, and 5 years, and the period may be shorter or longer than the exemplified period.

In the disclosure, a subject (e.g., a patient) having thyroid eye disease (TED) may receive a treatment of the present disclosure (e.g., a medicament, a pharmaceutical composition, a method, or the like) two weeks after receiving a previous administration of the treatment. In some embodiments, the subject (e.g., patient) can receive the antiIL-6 receptor antibody (e.g., satralizumab) or antigen binding fragment thereof contained in a medicament or a composition of the present disclosure via subcutaneous administration route.

In the disclosure, a subject (e.g., a patient) having thyroid eye disease (TED) may receive a treatment of the present disclosure (e.g., a medicament, a pharmaceutical composition, a method, or the like) four weeks after receiving a previous administration of the treatment. In some embodiments, the subject (e.g., patient) can receive the anti-IL-6 receptor antibody (e.g., satralizumab) or antigen binding fragment thereof contained in a medicament or a composition of the present disclosure via subcutaneous administration route.

In the disclosure, a subject (e.g., a patient) having thyroid eye disease (TED) may receive a treatment of the present disclosure (e.g., a medicament, a pharmaceutical composition, a method, or the like), e.g., every two weeks (Q2W) for three times (i.e., at time zero and again at 2 weeks and 4 weeks), and thereafter every four weeks (Q4W). In some embodiments, the subject (e.g., patient) can receive the anti-IL-6 receptor antibody (e.g., satralizumab) or antigen binding fragment thereof contained in a medicament or a composition of the present disclosure via subcutaneous administration route.

In addition to a treatment of TED in a subject of the present disclosure, the present disclosure is also used for prevention of TED in the subject. In the present disclosure, prevention of TED includes but is not limited to reducing a risk of relapse of, delaying relapse of, reducing a frequency of relapse of, or reducing severity of relapse of, or reducing the need for rescue therapy for relapse of TED.

An anti-IL-6 receptor antibody or antigen binding fragment thereof used in the present disclosure binds to an IL-6 receptor, inhibits the binding of IL-6 to an IL-6 receptor, blocks signal transduction by IL-6, and inhibits the biological activities of IL-6.

An anti-IL-6 receptor antibody used in the present disclosure can be obtained using known methods. In particular, an anti-IL-6 receptor antibody used in the present disclosure is preferably a monoclonal antibody derived from a mammal. Monoclonal antibodies derived from a mammal include those produced by a hybridoma and those produced by a host that has been transformed with an expression vector containing an antibody gene using genetic engineering methods.

Preferred examples of an “IL-6 receptor antibody” in the present disclosure include humanized anti-IL-6 receptor antibodies produced by modifying the variable and constant regions of tocilizumab, specifically, antibodies that comprise a heavy-chain CDR1 comprising the amino acid sequence of SEQ ID NO: 5, a heavy-chain CDR2 comprising the amino acid sequence of SEQ ID NO: 6, a heavy-chain CDR3 comprising the amino acid sequence of SEQ ID NO: 7, a light-chain CDR1 comprising the amino acid sequence of SEQ ID NO: 8, a light-chain CDR2 comprising the amino acid sequence of SEQ ID NO: 9, and a light-chain CDR3 comprising the amino acid sequence of SEQ ID NO: 10.

More preferred antibodies in the present disclosure include antibodies that comprise a heavy-chain variable region comprising the amino acid sequence of SEQ ID NO: 1 and a light-chain variable region comprising the amino acid sequence of SEQ ID NO: 2. Still more preferred are antibodies that comprise a heavy chain comprising the amino acid sequence of SEQ ID NO: 3 (heavy chain of satralizumab (generic name); SA237 (private name)) and a light chain comprising the amino acid sequence of SEQ ID NO: 4 (light chain of satralizumab). Satralizumab (private name: SA237) is particularly preferred.

Governmental marketing approval of satralizumab has been obtained in many countries including Japan, United States, and Europe based on the indication “prevention of relapses of neuromyelitis optica spectrum disorder (including neuromyelitis optica)”. The safety profiles identified during the international joint phase III clinical trials (SA-307JG/BN40898 study and SA-309JG/BN40900 study) targeting a population of patients with neuromyelitis optica spectrum disorder (NMOSD) and/or neuromyelitis optica (NMO) were mostly favorable. No death case was reported. The percentage of patients who experienced severe adverse events in the satralizumab group was about the same as that in the placebo group. There was no big difference between the two groups on the frequency of adverse events that led to discontinuation of administration of the test drug, or on the frequency of adverse events that led to drug withdrawal. Safety profiles were similar between the SA-309JG study, which was a single-agent test, and the SA-307JG study, which was a combined test with preexisting therapy (oral steroids and/or immunosuppressive agents).

Such antibodies can be obtained according to the methods described in WO2010/035769, WO2010/107108, WO2010/106812, and such. Specifically, antibodies can be produced using genetic recombination techniques known to those skilled in the art, based on the sequence of the above-mentioned IL-6 receptor antibody (see, for example, Borrebaeck C A K and Larrick J W, THERAPEUTIC MONOCLONAL ANTIBODIES, Published in the United Kingdom by MACMILLAN PUBLISHERS LTD, 1990). A recombinant antibody can be obtained by cloning a DNA encoding the antibody from a hybridoma or an antibody-producing cell such as an antibody-producing sensitized lymphocyte, inserting the DNA into an appropriate vector, and introducing the vector into a host (host cell) to produce the antibody.

Such antibodies can be isolated and purified using isolation and purification methods conventionally used for antibody purification, without limitation. For example, the antibodies can be isolated and purified by appropriately selecting and combining column chromatography, filtration, ultrafiltration, salting-out, solvent precipitation, solvent extraction, distillation, immunoprecipitation, SDS-polyacrylamide gel electrophoresis, isoelectric focusing, dialysis, recrystallization, and such.

The antibodies used in the present disclosure may be conjugate antibodies that are bound to various molecules such as polyethylene glycol (PEG), radioactive substances, and toxins. Such conjugate antibodies can be obtained by chemically modifying the obtained antibodies. Methods for antibody modification have been already established in this field. Accordingly, the term “antibody” in the present disclosure encompasses such conjugate antibodies.

The antibodies used in the present disclosure may be antibody fragments (also referred to as an antigen binding fragment of the antibody) or modified products thereof, as long as they can be suitably used in the present disclosure. For example, antibody fragments include Fab, F(ab′)2, Fv, and single chain Fv (scFv) in which the Fvs of the H and L chains are linked via an appropriate linker.

Specifically, the antibody fragments are produced by treating antibodies with enzymes such as papain or pepsin, or alternatively, by constructing genes encoding these antibody fragments and introducing them into expression vectors, and then expressing the vectors in appropriate host cells (see, for example, Co, M. S. et al., J. Immunol. (1994) 152, 2968-2976; Better, M. & Horwitz, A. H., Methods in Enzymology (1989) 178, 476-496; Plueckthun, A. & Skerra, A., Methods in Enzymology (1989) 178, 497-515; Lamoyi, E., Methods in Enzymology (1989) 121, 652-663; Rousseaux, J. et al., Methods in Enzymology (1989) 121, 663-666; and Bird, R. E. et al., TIBTECH (1991) 9, 132-137).

An scFv can be obtained by linking the H-chain V region and the L-chain V region of an antibody. In this scFv, the H-chain V region and the L-chain V region are linked via a linker, preferably via a peptide linker (Huston, J. S. et al., Proc. Natl. Acad. Sci. USA (1988) 85, 5879-5883). The V regions of the H and L chains in an scFv may be derived from any of the antibodies described above. Peptide linkers for linking the V regions include, for example, an arbitrary single chain peptide consisting of 12 to 19 amino acid residues.

A DNA encoding an scFv can be obtained by amplifying a DNA portion that encodes the desired amino acid sequence in template sequences with PCR using a primer pair which defines the termini of the portion, wherein a DNA encoding an H chain or an H-chain V region and a DNA encoding an L chain or an L-chain V region of the aforementioned antibodies are used as the templates, and then further amplifying the amplified DNA portion with a DNA that encodes a peptide linker portion and a primer pair that defines both ends of the linker so that it may be linked to each of the H and L chains.

Once an scFv-encoding DNA has been prepared, an expression vector comprising the DNA and a host transformed with the expression vector can be obtained according to conventional methods. In addition, an scFv can be obtained according to conventional methods by using the host.

Similar to the above, the antibody fragments can be produced by obtaining their genes, expressing them, and then using a host.

In the present disclosure, “as an active ingredient” means that the ingredient is contained in the pharmaceutical composition as a primal active ingredient, and the content thereof is not limited unless specifically indicated, as long as the antibodies or antigen binding fragments thereof used for the present disclosure are included as medicinal ingredients.

The dose of an anti-IL-6 receptor antibody or antigen binding fragment thereof contained in a medicament or a composition of the present disclosure is not particularly limited, and examples include 50 to 800 mg of antibody per administration, preferably 60 to 240 mg of antibody, and more preferably 60 mg, 120 mg, 180 mg, or 240 mg of antibody per administration. In certain embodiments of the present disclosure, a dosage unit of an anti-IL-6 receptor antibody or antigen binding fragment thereof contained in a medicament or a composition of the present disclosure (e.g., satralizumab) is administered to the subject. Each dosage unit can contain a fixed amount (e.g, 60 mg, 120 mg, 180 mg, or 240 mg) of the anti-IL-6 receptor antibody or antigen binding fragment. The dose of an anti-IL-6 receptor antibody or antigen binding fragment thereof contained in a medicament or a composition of the present disclosure may vary depending on the patient's body weight. In certain embodiments of the present disclosure, a suitable dose of the anti-IL-6 receptor antibody or antigen binding fragment for a subject with a body weight of less than 40 kg is 60 mg or 120 mg; a suitable dose for a subject with a body weight between 40 kg and 100 kg is 120 mg or 180 mg; and a suitable dose for a subject with a body weight of over 100 kg is 180 mg or 240 mg. A medicament or a composition comprising an anti-IL-6 receptor antibody or antigen binding fragment thereof of the present disclosure is administered to a subject via any route, including but not limited to subcutaneously, intravenously, intramuscularly, and by infusion. A preferred embodiment is subcutaneous administration.

In certain embodiments of the present disclosure, two or more sequential doses of an anti-IL-6 receptor antibody or antigen binding fragment thereof contained in a medicament or a composition of the present disclosure is administered to a subject during an initial period, wherein the doses administered during the initial period are spaced by a first dosing interval (also referred to the dosing interval that is shorter than the routine dosing interval), for example 20 weeks, 8 weeks, 4 weeks, or two weeks; and after the final dose administration of the initial period, waiting a second dosing interval that is longer than the first dosing interval and then administering a dose of an anti-IL-6 receptor antibody or antigen binding fragment thereof contained in a medicament or a composition of the present disclosure to a human patient, wherein optionally multiple consecutive doses are administered after the final dose administration of the initial period, and are spaced by the second dosing interval (also referred to as a “routine dosing interval”) that is not particularly limited except that it is longer than the first dosing interval. Examples of the second dosing interval include 1 day to 24 weeks, preferably 2 weeks to 8 weeks, more preferably 3 to 5 weeks, and even more preferably 4 weeks).

In certain embodiments of the present disclosure, an anti-IL-6 receptor antibody or antigen binding fragment thereof contained in a medicament or a composition of the present disclosure is administered to a subject every two weeks (Q2W) for three times, and thereafter every four weeks (Q4W).

The preferred administration schedule for an anti-IL-6 receptor antibody or antigen binding fragment thereof contained in a medicament or a composition of the present disclosure can be adjusted, for example, by appropriately extending the administration interval by monitoring the conditions of the disease and changes in the blood test values.

The present disclosure also provides an article of manufacture such as a kit, a device, and the like for use in a method of the present disclosure, which contains a pharmaceutical composition or a medicament of the present disclosure. The pharmaceutical composition or a medicament of the present disclosure comprises an IL-6 inhibitor as described herein. The article of manufacture may be packaged with an additional pharmaceutically acceptable carrier or medium, or instruction manual describing how to use the kits, etc.

In one embodiment, the article of manufacture comprises a container and a label on or a package insert associated with the container. Suitable containers include, for example, bottles, vials, syringes (including a prefilled syringe and an autoinjector), IV solution bags, etc. The containers may be formed from a variety of materials such as glass or plastic. In one embodiment, the container holds a composition which is by itself or combined with another composition effective for treating, preventing and/or diagnosing the condition and may have a sterile access port (for example the container may be a syringe, autoinjector, an intravenous solution bag or a vial having a stopper pierceable by a hypodermic injection needle). At least one active ingredient in the composition is an IL-6 inhibitor, preferably an anti-IL-6 receptor antibody, and more preferably satralizumab as described in the present disclosure.

In one embodiment, a device as the article of manufacture of the present disclosure as described above may be a prefilled syringe (PFS) optionally comprising a needle safety device (PFS-NSD) for injection via any administration route such as intravenously, subcutaneously, or the like, which comprises a fixed dose of an IL-6 inhibitor, preferably an anti-IL-6 receptor antibody, and more preferably satralizumab as described in the present disclosure in a pharmaceutically acceptable excipient. In another embodiment, the device may be an autoinjector (AI) for subcutaneous administration which comprises a fixed dose of an IL-6 inhibitor, preferably an anti-IL-6 receptor antibody, and more preferably satralizumab as described in the present disclosure in a pharmaceutically acceptable excipient. In certain embodiments, the device is a subcutaneous administration device, such as a prefilled syringe (PFS) and autoinjector (AI), which may comprise 60 mg, 120 mg, 180 mg, or 240 mg of satralizumab. In one embodiment, the subcutaneous administration device is a prefilled syringe comprising a needle safety device (PFS-NSD) which comprises 60 mg (for example 60 mg/mL) of satralizumab for delivering 60 mg or 180 mg doses to a subject. In another embodiment, the subcutaneous administration device is a prefilled syringe comprising a needle safety device (PFS-NSD) which comprises 120 mg (for example 60 mg/0.5 mL) of satralizumab for delivering 120 mg, 180 mg, or 240 doses to the subject. In other embodiments, the subcutaneous administration device is an autoinjector (AI) which comprises 120 mg, 180 mg, or 240 mg of satralizumab.

In the present disclosure, the label or package insert indicates that the pharmaceutical composition or medicament is used for treating the condition of choice. Moreover, the article of manufacture may comprise (a) a first container with a composition contained therein, wherein the composition comprises an IL-6 inhibitor, preferably an anti-IL-6 receptor antibody, and more preferably satralizumab as described above; and (b) a second container with a composition contained therein, wherein the composition comprises a further therapeutic agent. The article of manufacture in this embodiment of the disclosure may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Package Insert

The term “package insert” is used to refer to instructions customarily included in commercial packages of therapeutic products, that contain information about the indications, usage, dosage, administration, combination therapy, contraindications and/or warnings concerning the use of such therapeutic products.

A pharmaceutical composition or a medicament of the present disclosure can be formulated to produce freeze-dried formulations or solution formulations by mixing, if necessary, with suitable pharmaceutically acceptable carriers, vehicles, and such. The suitable pharmaceutically acceptable carriers and vehicles include, for example, sterilized water, physiological saline, stabilizers, excipients, antioxidants (such as ascorbic acid), buffers (such as phosphate, citrate, histidine, and other organic acids), antiseptics, surfactants (such as PEG and Tween), chelating agents (such as EDTA), and binders. Other low-molecular-weight polypeptides, proteins such as serum albumin, gelatin, and immunoglobulins, amino acids such as glycine, glutamine, asparagine, glutamic acid, aspartic acid, methionine, arginine, and lysine, sugars and carbohydrates such as polysaccharides and monosaccharides, and sugar alcohols such as mannitol and sorbitol may also be contained in the formulation. When preparing an aqueous solution for injection, physiological saline and isotonic solutions comprising glucose and other adjuvants such as D-sorbitol, D-mannose, D-mannitol, and sodium chloride may be used; and appropriate solubilizers such as alcohol (for example, ethanol), polyalcohols (such as propylene glycol and PEG), and nonionic surfactants (such as polysorbate 80, polysorbate 20, poloxamer 188, and HCO-50) may be used in combination. By mixing hyaluronidase into the formulation, a larger fluid volume can be administered subcutaneously (Expert Opin. Drug Deliv. 2007 July; 4(4): 427-40). Furthermore, syringes may be prefilled with the pharmaceutical composition of the present disclosure. Solution formulations can be prepared according to the method described in WO2011/090088.

If necessary, a pharmaceutical composition or a medicament of the present disclosure may be encapsulated in microcapsules (e.g., those made of hydroxymethylcellulose, gelatin, and poly(methylmethacrylate)), or incorporated into colloidal drug delivery systems (e.g., liposomes, albumin microspheres, microemulsions, nanoparticles, and nanocapsules) (see, for example, “Remington's Pharmaceutical Science 16th edition”, Oslo Ed. (1980)). Methods for preparing the pharmaceutical agents as controlled-release pharmaceutical agents are also known, and such methods may be applied to the pharmaceutical compositions of the present disclosure (Langer et al., J. Biomed. Mater. Res. 15: 267-277 (1981); Langer, Chemtech. 12: 98-105 (1982); U.S. Pat. No. 3,773,919; European Patent Application Publication No. EP 58,481; Sidman et al., Biopolymers 22: 547-556 (1983); and EP 133,988).

An anti-IL-6 receptor antibody or antigen binding fragment thereof contained in a medicament or a composition of the present disclosure can be administered to a patient via any appropriate route. For example, it can be administered to a patient intravenously by bolus injection or by continuous infusion, intramuscularly, intraperitoneally, intracerebrospinally, transdermally, subcutaneously, intraarticularly, sublingually, intrasynovially, orally, by inhalation, locally, or externally, for a certain period of time. Intravenous administration or subcutaneous administration is preferred. In certain embodiments of the present disclosure, an anti-IL-6 receptor antibody or antigen binding fragment thereof contained in a medicament or a composition of the present disclosure is administered to a subject subcutaneously.

All prior art references cited herein are incorporated by reference into the present specification.

### EXAMPLES

Herein below, the present disclosure will be specifically described with reference to the Examples, but it is not to be construed as being limited thereto.

### Example 1: Preparation of Satralizumab (SA237)

An antibody with the generic name satralizumab (and a private name of SA237), which is an IL-6 receptor antibody described in the patent document WO 2010/035769 as comprising a heavy chain having the amino acid sequence of SEQ ID NO: 26 (SEQ ID NO: 3 in the present specification) and a light chain having the amino acid sequence of SEQ ID NO: 29 (SEQ ID NO: 4 in the present specification)), was prepared according to the description of that patent document. The amino acid sequence of the heavy chain variable region is shown in SEQ ID NO: 1, and the amino acid sequence of the light chain variable region is shown in SEQ ID NO: 2. Using the prepared antibody, a subcutaneous administration preparation was prepared by the method described in the patent document WO 2011/090088.

### Example 2: Phase III, Randomized, Double-Masked, Placebo-Controlled, Multicenter Study to Evaluate the Efficacy, Safety, Pharmacokinetics, and Pharmacodynamics of Satralizumab in Participants with Moderate-to-Severe Thyroid Eye Disease

### 1. Introduction

### 1.1 Study Rationale

The purpose of this study is to assess the efficacy, safety, pharmacokinetics and pharmacodynamics of SC satralizumab, a recombinant, humanized anti-interleukin (IL) 6 receptor (IL 6R) monoclonal antibody, in patients with moderate-to-severe active and chronic inactive thyroid eye disease (TED), a complex orbital inflammatory autoimmune disorder. There remains an unmet medical need in patients with active moderate-to-severe TED for whom IV pulses of corticosteroids (CS) and/or teprotumumab (Unites States only) are the mainstay of treatment but are not universally successful or available and are associated with disease relapses and substantial side effects. There also remains an unmet medical need in patients with chronic inactive TED for whom there is no established medical treatment and surgical intervention remains the only option for treatment.

### 1.2 Benefit-Risk Assessment

Inflammation mediates the pathogenesis of TED, during the initial active phase as well as the subsequent chronic mixed inflammatory or fibrotic phase, as the disease continues to evolve to the final quiescent stage. Evidence from a number of in vitro and ex vivo experiments support an important role of IL-6 and its receptor in the disease, suggesting the potential of satralizumab as a novel drug for treatment of TED. Patients with active TED have higher levels of serum IL-6R than those with inactive TED (Slowik et al., 2012). IL-6 also increases the expression of TSHR in orbital fibroblast pre adipocytes (Dik et al., 2016). Polymorphisms in the IL-6 gene are thought to be involved in the predisposition to and development of TED (Anvari et al., 2010). Further, in vitro and ex vivo data have demonstrated a role for IL-6 in TED by showing that IL-6 expression from orbital tissue correlates with orbital tissue expansion (Hiromatsu et al., 2000). IL-6 has the ability to increase TSHR mRNA levels in cultured orbital fibroblasts of patients with TED. In addition, autoantibodies against TSHR increase IL-6 expression in orbital pre-adipocyte fibroblasts and IL-6 secretion by mature adipocytes (Kumar et al., 2010).

Clinical trial results from academic investigator led studies and series with systemic administration of anti-IL6R antibody, tocilizumab, support an important role of IL-6 and its receptor in TED (Perez-Moreiras et al. 2014; Perez Moreiras et al. 2018; Sanchez-Bilbao et al. 2020).

Furthermore, there is no established medical treatment for chronic inactive TED and surgical intervention remains the mainstay of treating patients who continue to suffer from visual impairment, diplopia or disfiguring proptosis and keratopathy, with patients needing to wait at least 6 months or longer after reaching the inactive phase of their disease before having any reconstructive or rehabilitative procedures (Bartalena et al. 2016).

Satralizumab has demonstrated efficacy and safety in its approved indication of NMOSD. In the two Phase III studies in NMOSD, satralizumab every 4 weeks (Q4W) as monotherapy (BN40900) or in combination with immunosuppressive therapies (ISTs) (BN40898) was generally safe and well tolerated by patients with NMOSD. Satralizumab has not yet been tested in patients with TED. However, it is expected that the safety profile of satralizumab in patients with TED would be consistent with its class effect as an IL-6R inhibitor. All necessary measures will be taken to closely monitor the safety of the participants enrolled in this study.

Taking into account the potential for efficacy in patients with moderate-to-severe TED for whom there is a high unmet medical need, the safety profile for satralizumab, and the risk-mitigation measures for this study, the benefit-risk ratio is expected to be acceptable for satralizumab in the treatment of TED.

### 2. Objectives, Estimands, and Endpoints

This study will evaluate the efficacy, safety, pharmacokinetics and pharmacodynamics of satralizumab compared with placebo in participants with moderate-to-severe TED.

Tables 1 and 2 present the primary and key secondary objectives for the study expressed using the estimand framework in accordance with the International Council for Harmonisation (ICH) E9(R1) statistical principles for clinical trials (ICH 2020). Table 3 presents the remaining objectives and corresponding endpoints.

TABLE 1
Primary Objective and Corresponding Estimands

| Primary Objective | Estimand Definition |
|---|---|
| To evaluate the efficacy of | Population: adult participants with TED (active disease for primary |
| subcutaneous | hypothesis) as defined by the study inclusion and exclusion criteria. |
| satralizumab compared | Variable: proportion of participants achieving ≥2 mm reduction in proptosis |
| with placebo at Week 24 | from baseline (Day 1) at Week 24 in the study eye, provided there is no |
| | deterioration of proptosis (≥2 mm increase) in the fellow eye |
| | Treatment (as randomized): |
| | Experimental: satralizumab SC 60 mg, 120 mg, or 180 mg (for |
| | bodyweight <40 kg, ≥40-≤100 kg, >100 kg, respectively), at |
| | Day 1, Week 2, and Week 4 (loading doses) and maintenance |
| | doses Q4W thereafter until Week 20Seven doses in total during |
| | the 24-week Part I period |
| | Control: placebo SC at Day 1, Week 2, and Week 4 (loading |
| | doses), and maintenance doses Q4W thereafter until Week 20. |
| | Seven doses in total during the 24-week Part I period |
| | Intercurrent events and handling strategies: |
| | Early discontinuation of study treatment due to AEs or lack of |
| | efficacy: treatment policy strategy |
| | Use of any prohibited systemic treatment or prohibited therapy for |
| | either eye (study eye or fellow eye): evaluated as non-responder |
| | Administration of rescue treatment in the study eye: evaluated as |
| | non-responder |
| | Orbital or ocular surgery for TED (e.g., orbital decompression, |
| | eyelid or strabismus surgery) in either eye (study eye or fellow |
| | eye): evaluated as non-responder |
| | Population-level summary: difference in proportions of proptosis |
| | responders at Week 24 between the satralizumab and placebo group |

TABLE 2
Key Secondary Objectives and Corresponding Estimands

| Key Secondary Objectives | Estimand Definition |
|---|---|
| To assess the efficacy of | Population: adult participants with TED (active and chronic |
| subcutaneous satralizumab | inactive disease) as defined by the study inclusion and |
| compared with placebo at Week 24 | exclusion criteria |
| based on proptosis response | Variable: proportion of participants achieving ≥2 mm |
| | reduction in proptosis from baseline (Day 1) at Week 24 in the |
| | study eye provided there is no corresponding deterioration in |
| | proptosis (≥2-point/mm increase) in the fellow eye |
| | Treatment: as defined above |
| | Intercurrent events and handling strategies: as in primary |
| | estimand definition |
| | Population-level summary: difference in proportions of |
| | proptosis responders in overall (active and chronic inactive) |
| | population at Week 24 between the satralizumab and |
| | placebo arm |
| To assess the efficacy of | Population: adult participants with TED (active disease) as |
| subcutaneous satralizumab | defined by the study inclusion and exclusion criteria |
| compared with placebo at Week 24 | Variable: Change from baseline (Day 1) in proptosis at |
| based on change in proptosis | Week 24 |
| | Treatment: as defined above |
| | Intercurrent events and handling strategies: intercurrent |
| | events as defined aboveA treatment policy strategy will be |
| | applied to all intercurrent events |
| | Population-level summary: least square mean difference |
| | between the satralizumab and placebo group |
| To assess the efficacy of | Population: adult participants with TED (active and chronic |
| subcutaneous satralizumab | inactive disease) as defined by the study inclusion and |
| compared with placebo at Week 24 | exclusion criteria |
| based on change in proptosis | Variable: change from baseline (Day 1) in proptosis at |
| | Week 24 |
| | Treatment: as defined above |
| | Intercurrent events and handling strategies: intercurrent |
| | events as defined aboveA treatment policy strategy will be |
| | applied to all intercurrent events |
| | Population-level summary: least square mean difference |
| | between the satralizumab and placebo group |
| To assess the efficacy of | Population: in subset of active TED participants as defined by |
| subcutaneous satralizumab compared | the study inclusion and exclusion criteria with baseline (Day 1) |
| with placebo at Week 24 based on | diplopia >0 in the study eye |
| reduction/improvement in diplopia | Variable: proportion of participants achieving ≥1 grade |
| | reduction/improvement in diplopia at Week 24 |
| | Treatment: as defined above |
| | Intercurrent events and handling strategies: as in primary |
| | estimand definition |
| | Population-level summary: difference in proportions of |
| | participants achieving ≥1 grade reduction/improvement in |
| | diplopia at Week 24 between the satralizumab and placebo |
| | group |
| To assess the efficacy of | Population: in subset of overall (active and chronic inactive) TED |
| subcutaneous satralizumab compared | participants as defined by the study inclusion and exclusion |
| with placebo at Week 24 based on | criteria with baseline (Day 1) diplopia >0 |
| reduction/improvement in diplopia | Variable: proportion of participants achieving ≥1 grade |
| | reduction/improvement in diplopia at Week 24 |
| | Treatment: as defined above |
| | Intercurrent events and handling strategies: as in primary |
| | estimand definition |
| | Population-level summary: difference in proportions of |
| | participants achieving ≥1 grade reduction/improvement in |
| | diplopia at Week 24 between the satralizumab and placebo |
| | group |
| To assess the efficacy of | Population: in subset of active TED participants as defined by |
| subcutaneous satralizumab compared | the study inclusion and exclusion criteria with motility-induced |
| with placebo at Week 24 based on | pain at baseline (Day 1) in the study eye |
| improvement in orbital pain | Variable: proportion of participants achieving absence of motility- |
| | induced pain at Week 24 |
| | Treatment: as defined above |
| | Intercurrent events and handling strategies: as in primary |
| | estimand definition |
| | Population-level summary: difference in proportions of |
| | participants with absence of motility-induced pain at Week 24 |
| | between the satralizumab and placebo group |
| To assess the efficacy of | Population: in subset of active TED participants as defined by |
| subcutaneous satralizumab compared | the study inclusion and exclusion criteria with spontaneous pain |
| with placebo at Week 24 based on | at baseline (Day 1) in the study eye |
| improvement in orbital pain | Variable: proportion of participants achieving absence of |
| | spontaneous pain at Week 24 |
| | Treatment: as defined above |
| | Intercurrent events and handling strategies: as in primary |
| | estimand definition |
| | Population-level summary: difference in proportions of |
| | participants with absence of spontaneous pain at Week 24 |
| | between the satralizumab and placebo group |

AE = adverse event; SC = subcutaneous; Q4W = Every 4 weeks; TED = thyroid eye disease.

TABLE 3
Other Secondary and Exploratory Objectives and Endpoints for Part I

Corresponding Endpoints

Other Secondary Objectives and Endpoints

| | |
|---|---|
| Efficacy Objectives | |
| To assess the efficacy of subcutaneous | Proportion of participants with a ≥6 point improvement in the |
| satralizumab compared with placebo at | Visual Functioning and Appearance subscale scores of the |
| Week 24 based on health-related quality | GO-QoL from baseline at Week 24 |
| of life | |
| To further assess the efficacy of | Proportion of participants with active disease achieving |
| subcutaneous satralizumab at Week 24 | overall response at Week 24, defined as a ≥2-point reduction |
| | in CAS, AND a ≥2 mm reduction in proptosis from baseline |
| | (Day 1) in the study eye, provided there is no corresponding |
| | deterioration in CAS or proptosis (≥2 point/mm increase) in |
| | the fellow eye |
| | Proportion of participants with active disease achieving a ≥2- |
| | point reduction in CAS in the study eye from baseline (Day 1) |
| | to Week 24 |
| | Proportion of participants achieving a CAS value of 0 or 1 in |
| | the study eye (indicating no or minimal inflammation, |
| | respectively) at Week 24 ^{a} |
| | Propotion of participants with a ≥10 point improvement in the |
| | OSDI overall scores across all levels of baseline severity at |
| | Week 24 |
| | Change from baseline (Day 1) in the OSDI Ocular |
| | Symptoms, and Vision-related Function subscale scores at |
| | Week 24 |
| | Change in Oxford corneal staining scores from baseline (Day |
| | 1) at Week 24^{b} |
| To assess the efficacy of subcutaneous | Proportion of participants achieving ≥2 mm reduction in |
| satralizumab at Week 48 in those | proptosis at Week 48 in the study eye (provided there is no |
| participants who are proptosis non- | corresponding deterioration in proptosis [≥2 mm increase] in |
| responders at Week 24 | the fellow eye). |
| | Proportion of participants achieving overall response at Week |
| | 48, defined as a ≥2-point reduction in CAS, AND a ≥2 mm |
| | reduction in proptosis from baseline (Day 1) in the study eye, |
| | provided there is no corresponding deterioration in CAS or |
| | proptosis (≥2 point/mm increase) in the fellow eye |
| | Proportion of participants achieving a ≥2-point reduction in |
| | CAS in the study eye from baseline (Day 1) to Week 48 |
| | In the subset of participants with baseline (Day 1) diplopia |
| | (>0): Proportion of participants achieving Grade ≥1 |
| | reduction/improvement in diplopia at Week 48. |
| | Proportion of participants with a ≥6 point improvement in the |
| | Visual Functioning and Appearance subscale scores of the |
| | GO-QoL at Week 48 |
| | Change in proptosis from baseline (Day 1) to Week 48 |
| | Proportion of participants requiring surgical intervention for |
| | TED throughout the course of study |
| To assess the efficacy of subcutaneous | Proportion of participants with worsening of proptosis by ≥2 |
| satralizumab at Week 48 in those | mm from Week 24 to Week 48 |
| participants who are proptosis responders | Proportion of participants with worsening of proptosis by ≥2 |
| at Week 24 | mm from baseline (Day 1) to Week 48 |
| | Change in proptosis from Week 24 to 48 |
| | Change in CAS from Week 24 to 48 |
| | Proportion of participants with a ≥6 point improvement in the |
| | Visual Functioning and Appearance sub-scale scores of the |
| | GO-QoL at Week 48 |
| | Change in proptosis from baseline (Day 1) to Week 48 |
| | Change in CAS from baseline (Day 1) to Week 48 |
| | Proportion of participants requiring surgical intervention for |
| | TED throughout the course of study |
| Safety Objective | |
| To evaluate the safety and tolerability | Incidence, seriousness, and severity of AEs, with |
| of satralizumab | severity determined according to NCI CTCAE V5 |
| | Change from baseline (Day 1) in vital signs, targeted |
| | clinical laboratory test results, ECG parameters, and |
| | body weight |
| Pharmacokinetic Objective | |
| To characterize the satralizumab PK | Serum concentration of satralizumab at specified |
| profile by evaluating plasma exposure | timepoints |
| over 24 weeks | Estimates of primary PK parameters (e.g., CL/F and |
| | V/F) and secondary PK parameters (e.g., AUC) |
| | derived using popPK modeling |

Exploratory Objectives and Endpoints

| | |
|---|---|
| Efficacy Objectives | |
| To further assess the efficacy of | Change in CAS from baseline (Day 1) to Week 24 |
| subcutaneous satralizumab compared | Proportion of participants requiring orbital |
| with placebo at Week 24 | decompression surgery or any form of reconstructive |
| | surgery from baseline (Day 1) to Week 24 |
| | Change in the motility component on the Clinical |
| | Measures of Severity scale (based on EUGOGO |
| | consensus) from baseline (Day 1) to Week 24 |
| | Proportion of participants with improvement for each |
| | component of the Clinical Measures of Severity scale |
| | (based on EUGOGO consensus) from baseline |
| | (Day 1) to Week 24 |
| | Proportion of participants at each overall response |
| | level at Week 24 (high responders, responders, low |
| | responders and non-responders ^{a}) |
| | Evaluation of each individual component of CAS at |
| | Week 24 |
| | In the subset of participants with baseline (Day 1) |
| | orbital MR imaging, change in extraocular muscle and |
| | fat volume from baseline (Day 1) to Weeks 24 |
| | Change in BCVA from baseline at Weeks 24 |
| To assess the efficacy of subcutaneous | Proportion of participants achieving ≥2 mm reduction |
| satralizumab compared with placebo in | in proptosis from baseline (Day 1) at week 24 |
| participants with chronic inactive TED at | Change in proptosis from baseline to week 24 |
| Week 24 | Proportion of participants with a ≥6 point improvement |
| | in the Visual Functioning and Appearance sub-scale |
| | scores of the GO-QoL at Week 24 |
| | Proportion of participants achieving ≥1 grade |
| | reduction/improvement in diplopia at week 24 |
| To further assess the efficacy of | Change in CAS from baseline (Day 1) to Week 48 |
| subcutaneous satralizumab compared | In the subset of participants with active TED at |
| with placebo at Week 48 | baseline (Day 1): Time to first achieving CAS value of |
| | 0 or 1 in the study eye (indicating no or minimal |
| | inflammation, respectively) from baseline (Day 1) to |
| | Week 48 |
| | Proportion of participants requiring orbital |
| | decompression surgery or any form of reconstructive |
| | surgery from baseline (Day 1) at Week 48 |
| | Change in the motility component on the Clinical |
| | Measures of Severity scale (based on EUGOGO |
| | consensus) from baseline (Day 1) to Week 48 |
| | Proportion of participants with improvement for each |
| | component of the Clinical Measures of Severity scale |
| | (based on EUGOGO consensus) from baseline |
| | (Day 1) to Week 48 |
| | Proportion of participants at each overall response |
| | level at Week 48 (high responders, responders, low |
| | responders and non-responders ^{a}) |
| | Evaluation of each individual component of CAS at |
| | Week 48 |
| | In the subset of participants with baseline (Day 1) |
| | orbital MR imaging, change in extraocular muscle and |
| | fat volume from baseline (Day 1) to Weeks 48 |
| | Change in BCVA from baseline at Weeks 48 |
| | Proportion of participants with a ≥10 point |
| | improvement in the OSDI overall scores across all |
| | levels of baseline severity at Week 48 |
| | Change from baseline (Day 1) in the OSDI Ocular |
| | Symptoms, and Vision-related Function subscale |
| | scores at Week 48 |
| | Change in Oxford corneal staining scores from |
| | baseline (Day 1) at Week 48^{b} |
| | Proportion of participants requiring medical therapy, |
| | orbital decompression surgery or any form of |
| | reconstructive surgery for TED from Week 48 through |
| | Week 72 |
| Pharmacodynamic Objective | |
| To evaluate the pharmacodynamics of | Absolute values and change from baseline in serum |
| satralizumab | levels of biomarkers IL-6 and soluble IL-6 receptor |
| Immunogenicity Objective | |
| To evaluate the immune response to | Prevalence of ADAs at baseline (Day 1) and incidence |
| satralizumab | of ADAs during the study |
| To evaluate potential effects of ADAs on | Relationship between ADA status and efficacy, safety, |
| efficacy, biomarker, safety, and PK | or PK endpoints |
| endpoints | |
| Biomarker Objectives | |
| To evaluate potential relationships | Relationship between selected covariates and |
| between drug exposure and the efficacy | exposure to satralizumab |
| and safety of satralizumab | Relationship between serum concentration or PK |
| | parameters for satralizumab and efficacy endpoints, |
| | PD biomarkers, and safety endpoints |
| To identify and/or evaluate biomarkers | Relationship between biomarkers in circulation |
| that are predictive of response to | including thyroid receptor auto-antibodies and efficacy, |
| satralizumab, can provide evidence of | safety, PK, immunogenicity, or other biomarker |
| satralizumab activity, or can increase the | endpoints |
| knowledge and understanding of disease | |
| biology | |

ADA = anti-drug antibody; AE = adverse event; AUC = area under the concentration-time curve; CAS = clinical activity score; EUGOGO = European Group on Graves Orbitopathy; GO-QoL = Graves' Ophthalmopathy Quality of Life; CL = clearance; IL-6 = interluekin-6; NCI CTCAE = National Cancer Institute Common Terminology Criteria for Adverse Events, Version 5.0; OSDI = ocular surface disease index; PK = pharmacokinetic; popPK = population pharmacokinetics; TED = thyroid eye disease; V/F = apparent volume of distribution.
^{a} Participants will be classified into one of four response categories (CAS changes are measured on a 7-item scale) for the exploratory endpoint. Participants that meet criteria for several response categories will be allocated to the highest possible category. The handling of missing values will be discussed in the SAP.
High responders are participants who had a reduction in both proptosis (2 mm or more) and CAS (2 points or more) from baseline (Day 1) in the study eye, and no deterioration in the fellow eye (i.e., increase in CAS 2 points or more or increase in proptosis 2 mm or more).
Responders are participants who had a reduction in either proptosis (2 mm or more) or (but not “and”) CAS (2 points or more) from baseline (Day 1) in the study eye (but no worsening in the other respective endpoint (proptosis or CAS as defined above in the study eye, and no deterioration in the fellow eye [as defined above]).
Low responders are participants who had a reduction in either proptosis (<2 mm) or (but not “and”) CAS (<2 points ) from baseline (Day 1) in the study eye (but no worsening in the other respective endpoint (proptosis or CAS as defined above in the study eye, and no deterioration in the fellow eye [as defined above]).
Non-Responders are participants who did not fit into any of the above categories
^{b}The scale in the Oxford grading scheme chart categorize corneal staining into 6 groups according to severity: 0 = absent; I = minimal; II = mild; III = moderate; IV = marked; and V = severe. The observer/examiner will compare the overall appearance of the participant's corneal staining. The observer/examiner should select the appropriate grade that best represents the state of corneal staining. The staining score will be recorded for the exposed interpalpebral cornea and conjunctiva.

### 3. Study Design

### 3.1 Overall Design

The study includes the following phases:
Screening period (up to 4 weeks long): During the screening period, individuals' eligibility will be evaluated for study participation.
Placebo-controlled treatment periods: this will be split into two parts:
   Part I: Weeks 1-24; with study treatment administered until Week 20, and primary endpoint assessed at Week 24.
   Part II: Weeks 24-48, with study treatment administered until Week 44, and an analysis conducted at Week 48.

The final visit of Part I coincides with the first visit of Part II (Week 24). At this visit, response to treatment will be assessed. Proptosis non-responders from either arm will receive satralizumab treatment in Part II. Proptosis responders from either arm will be re-randomized to satralizumab or placebo in Part II, regardless of their original treatment assignment.
Follow-up period: Weeks 48-72. Participants will be assessed every 2 months through telephone calls.

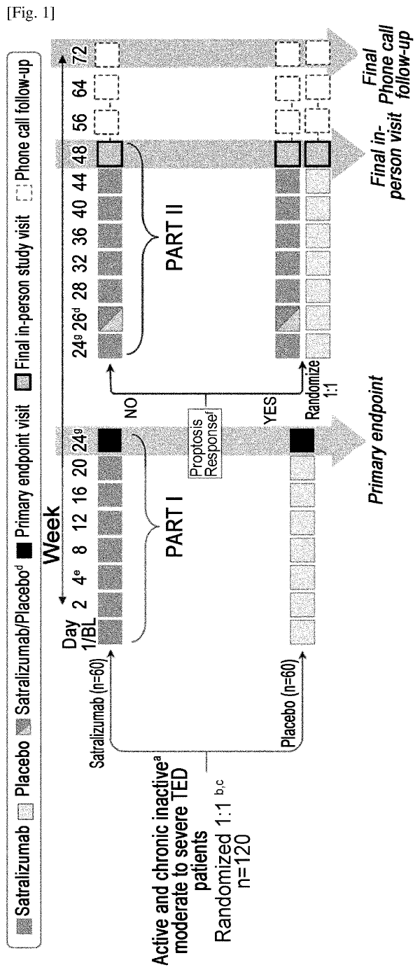

The primary endpoint will be assessed at Week 24, followed by an analysis at Week 48, and a final analysis will be conducted at Week 72. FIG. 1 presents an overview of the study design.

### 3.1.1 Overview of Study Design

Approximately 120 participants who meet the study eligibility criteria will be randomized on Day 1 in a 1:1 ratio (stratified by current smoking status [current smoker, including the use of e-cigarettes vs. former smoker vs. never smoked] and TED disease activity status [active vs. inactive] to receive seven SC doses of either satralizumab (with dosing based on body weight [<40 kg: 60 mg, between 40 and 100 kg: 120 mg, or >100 kg: 180 mg]) or matching placebo. Patients with chronic inactive TED will be capped at 25% of the total enrollment.

Only one eye will be assigned as the study eye. If both eyes are considered eligible, the eye with the worse severity, as assessed at screening by evaluation of proptosis, diplopia, pain and other clinical measures of severity, will be selected as the study eye unless the investigator deems the other eye to be more appropriate for treatment in the study. If both eyes have the same severity of disease, the investigator will chose the most appropriate eye as the study eye.

During the study, efficacy measures are investigator-assessed proptosis (measured using a Hertel exophthalmometer instrument provided by the Sponsor for consistency in measurement), and Clinical Activity Score (CAS; 7-item scale). The same observer should be used at each evaluation (except when strictly unavoidable) for the entire duration of the study. Disease severity will be assessed using clinical measures of severity based on the European Group on Grave's Orbitopathy (EUGOGO) consensus statement (Bartalena et al. 2021). Health-related quality of life will be assessed using the Graves' Ophthalmopathy Quality of Life (GO-QoL) questionnaire. Signs of ocular surface/corneal involvement will be assessed using fluorescein staining of the ocular surface and graded on a slit-lamp biomicroscope using the Oxford Grading Scheme Chart. Symptoms of dry eye affecting the ocular surface and their impact on visual function will be assessed with the Ocular Surface Disease Index (OSDI) questionnaire. In addition, standard ophthalmological examination assessing visual acuity, eyelid position, pupillary reaction and retina will be performed. Safety will be monitored via adverse events, vital signs, laboratory parameters, ECGs, and concomitant medication use.

### 3.1.1.1 Part I Period

During Part I, eligible participants will be randomized to receive either satralizumab (60, 120, or 180 mg based on body weight) or matching placebo on Day 1 and at Weeks 2, 4, 8, 12, 16, and 20, with a final visit of the Part I period at Week 24. The treatment which participants receive will be assigned via interactive voice or webbased response system (IxRS).

### 3.1.1.2 Part II Period

After the 24-week treatment period in Part I, participants will enter a second treatment period called Part II. The treatment which participants receive in Part II of the study will be assigned via IxRS and is determined by their proptosis response (PR) at the end of Part I (Week 24 primary endpoint):
Participants who are proptosis non-responders at Week 24 will receive satralizumab treatment during Part II (Weeks 24-44).
Participants who are proptosis responders at Week 24 will be re-randomized in a 1:1 ratio to receive SC satralizumab (with dosing based on body weight [<40 kg: 60 mg, between 40 and 100 kg: 120 mg, or >100 kg: 180 mg]) or matching placebo (Weeks 24-44), regardless of their original treatment assignment in Part I.

During Part II of the study, the participants and the study staff will remain masked to the treatment the participants received during Part I of the study.

Due to the study design, participants who received placebo during Part I and receive satralizumab in Part II will receive a loading dose of Satralizumab at Week 26; participants who received satralizumab in Part I and receive satralizumab in Part II do not require a loading dose and will receive placebo at Week 26.

The final visit at Week 48 in Part II (4 weeks after the final dose) is the last in-person visit of the study; subsequent follow up visits will be conducted by telephone.

### 3.1.1.3 Follow-Up Period

After Week 48, participants will enter a 6-month telephone follow up period. The patient will be contacted every 2 months (i.e., at Weeks 56, 64 and 72) for a telephone follow up visit, to review adverse events, concomitant treatments, and any treatments or surgery received for TED.

If participants choose to visit the study site in-person during the ‘telephone follow-up period’, the same information that would be collected from telephone conversation will be obtained; no other data will be recorded on the eCRF.

### 3.2 Rationale for Study Design

A multicenter, randomized, double-masked, placebo-controlled design was selected to minimize the bias in the evaluation of SC satralizumab as a treatment for patients with moderate-to-severe TED.

To ensure the safety of all participants during the study, several safety assessments have been included (e.g., regular ophthalmic monitoring, adverse event monitoring [ocular and systemic], and laboratory safety tests).

### [0088]3.2.1 Rationale for Study Population

As mild TED is often self-limiting and can spontaneously improve, the study will enroll patients with moderate-to-severe (active and chronic inactive) disease that will be defined according to the EUGOGO criteria for severity assessment of two or more of the following:
Lid retraction: 2 mm or more,
Moderate or severe soft tissue involvement,
Exophthalmos: 3 mm or more above normal.
Inconstant or constant diplopia.

Eligible participants must be adults, aged 18 years or older at baseline (Day 1). Pediatric population will not be enrolled into this study because TED is a rare condition in children and when it does occur, it is mild in severity and self-limiting. In addition, TED in children is largely amendable to restoration of thyroid function and local supportive therapy.

Study participants will be required to be euthyroid or mildly hyperthyroid or hypothyroid at the baseline visit, based on the measurement of normal free thyroid hormone concentrations. This criterion is meant to eliminate a potential bias due to the impact of systemic hyper- or hypo-thyroidism on the satralizumab versus placebo comparison.

The proposed study will include patients with active and chronic inactive TED, and patients with chronic inactive TED will be capped at 25% of the enrollment. Although IL 6 levels in patients with chronic inactive TED are higher compared with healthy controls, they are lower when compared with patients with active TED. Therefore, it is anticipated that there will be greater efficacy observed in patients with active TED. The study is fully powered for the treatment comparison in the patient population with active TED. If satralizumab demonstrates efficacy in patients with active TED, it is expected to also be beneficial in patients with chronic inactive disease, however, with a smaller difference between satralizumab and placebo group due to lower target expression and change in the character of disease from purely inflammatory to the inflammatory/fibrotic stage. Capping the number of patients with chronic inactive TED at 25% will allow the study to remain powered for assessment of efficacy in the patient population with active TED alone, and also in the overall patient population (active and chronic inactive TED). The totality of data from the proposed design will allow the evaluation of the consistency of treatment benefits between patients with active and chronic inactive TED.

### 3.2.2 Rationale for Control Group

In this study, placebo will be used as the comparator to evaluate the efficacy and safety of SC satralizumab from baseline (Day 1) through Week 24 (Part I); placebo will also be the comparator used in Part II, where proptosis responders at Week 24 will be re randomized to satralizumab or placebo and treated through Week 44.

Globally, systemic CSs are used as an off-label treatment option in TED, and remain the mainstay of management for active, moderate-to-severe TED because of their rapid anti-inflammatory and immunosuppressive effects in some patients at high doses (Gillespie et al. 2012; Bartalena et al. 2016). Even in the United States where teprotumumab has been approved in 2020, off-label oral or IV CS therapy continues to be used as the first line therapy (followed by off-label SC or IV tocilizumab or radiation as a second line option at some clinics), due to less tolerability of teprotumumab treatment, and complexities in coordinating care with infusion centers. The treatment response to CSs is not always consistent, as a large proportion of patients (up to 40%) still experience disease progression (Bartalena et al. 2012) or do not respond to CS therapy at all requiring one or more surgical interventions (Zang et al. 2011). CSs may even mediate adipogenesis, possibly contributing to the expansion of orbital fat in TED and worsening the disease (Draman and Ludgate 2016), and long-term side effects associated with high dose CS treatment can include hepatotoxicity, Cushing syndrome, osteoporosis (of particular concern in women), increased risk of serious infections, glaucoma, diabetes mellitus, and other severe consequences. Given the substantial burden from the use of CSs, the Sponsor considers placebo as a comparator for this study. In addition, the route of administration of CSs would limit masking to treatment assignment if used as a comparator in the study. To address any ethical considerations and mitigate any potential risks to study participants, all enrolled participants will be eligible for rescue intervention from Week 4 based on protocol-defined criteria as described in Section 5.1.2.

Furthermore, in patients with chronic inactive TED, there is currently no established medical treatment. Inactive disease is less amenable to available medical treatments that are used for the active stage. Surgical intervention remains the mainstay of treating patients with chronic inactive TED but is often unsuccessful or has to be repeated in a series of procedures over a long period of time. Therefore, the use of CSs would be less suitable in this population.

This study will be a multicenter, global Phase III study, and as satralizumab is administered subcutaneously, teprotumumab IV infusions would not be an acceptable comparator for multiple reasons, including the different route of delivery and the fact that it is not approved in countries outside of the United States for treatment of TED. Additionally, this study is recruiting patients with both active and chronic inactive TED, and the only robust data available with teprotumumab in TED are in patients with active disease; there is currently only minimal evidence on the drug's effectiveness in the population with chronic inactive TED. Even in patients with active TED, approximately 40% of teprotumumab treated patients who initially responded do experience disease relapse as their disease transitions from active to the chronic inactive phase. Serious and frequent adverse events, some occurring in up to approximately 25% patients treated with teprotumumab, such as hearing impairment, elevated blood glucose levels, alopecia, nausea, muscle spasm, diarrhea and changes in menstruation further limit the use for only a subset of patients who accept these risks. An additional challenge with teprotumumab is that its long-term safety profile has not yet been established (Highland et al. 2021; Yu et al. 2021; Belinsky et al. 2022; Chow and Silkiss 2022; Sears et al. 2022; Ding et al. 2022). Teprotumumab route of delivery would also present with masking related challenges limiting its consideration as a comparator. Overall, the limited efficacy data, the relapse rates, the current safety profile associated with teprotumumab, make this agent unsuitable for use as a comparator because it is not well established as a standard of care, and many questions including its long-term efficacy and safety profile remain unanswered.

Despite treatment with the currently available options, patients with TED do not fully recover, continue to suffer and ultimately have to undergo surgery or a series of surgical procedures to correct the signs and symptoms of TED. Due to the limitations of currently available treatments in TED, placebo is considered as the appropriate comparator in this study (in Part I and re-randomized participants in Part II) to evaluate the efficacy, safety, pharmacokinetics, and pharmacodynamics of SC satralizumab. To ensure patient's safety, all study participants will be eligible for rescue intervention from Week 4 based on protocol defined criteria as described in Section 5.1.2.

### 3.2.3 Rationale for Primary Endpoint Selection

The primary objective of the proposed Phase III study is to evaluate the efficacy of satralizumab compared with placebo and the proposed primary efficacy endpoint is investigator-assessed PR, defined as the proportion of participants achieving a 2 mm or more reduction in proptosis from baseline (Day 1) at Week 24 in the study eye (i.e., the eye with greater proptosis) provided there is no corresponding deterioration in proptosis (2 mm or more increase) in the fellow eye. The changes in PR over time to support the primary endpoint variable will be measured with a Hertel exophthalmometer instrument provided by the Sponsor and the same observer should be used at each evaluation (except when strictly unavoidable) for the entire duration of the study for consistency in measurement.

Some of the clinical symptoms and signs of TED can be explained mechanically by the discrepancy between the increased volume of the orbital tissues and the fixed volume of the bony orbit. The expanded orbital tissues displace the globe forward and impede venous outflow from the orbit. These changes, combined with the local production of cytokines and other mediators of inflammation, result in pain, proptosis, periorbital edema, conjunctival injection, and chemosis among other signs and symptoms.

One published, randomized, double-masked, placebo-controlled trial that investigated the efficacy of anti-IL-6R monoclonal antibody (tocilizumab) in patients with active, moderate-to-severe CS-resistant TED demonstrated a reduction in proptosis at Week 16 in 93% of patients compared with 47% on placebo (Perez-Moreiras et al. 2018). Efficacy was also demonstrated with a change in CAS of at least 2 points from baseline to Week 16, which was achieved in 93% of patients receiving tocilizumab versus 59% receiving placebo. Another study, which was prospective, interventional, and non-randomized investigated the use of tocilizumab 8 mg/kg IV Q4W for the treatment of CS refractory, active (CAS=4 or more) TED in 18 patients. Results demonstrated a reduction in proptosis in 72% of patients, with 83% experiencing improvement in extraocular motility, and resolution of diplopia in 54% of patients. All patients had a significant progressive CAS improvement (mean CAS score reduction 5.89 [+/−1.4] points, p<0.00027). The improvement in the CAS was stable in all cases up to 27 months, with a minimum follow-up period of 9 months (Perez-Moreiras et al. 2014).

The disruption of the inflammatory cascade with the corresponding decrease in adipogenesis and extracellular matrix formation mediated by anti-IL-6R blockade with satralizumab will result in improvement of proptosis and CAS signs and symptoms. Unlike CS IV pulses and teprotumumab IV treatment options, which are given over a short-term, leading to a significant rate of TED relapse, continued satralizumab SC dosing is expected to provide an improved benefit-risk profile with improved control of inflammation over the longer term, potentially reducing the need for surgical interventions or secondary treatments.

Based on the heterogeneity of the study population that includes patients with active and chronic inactive TED, the Sponsor proposes PR as the appropriate primary efficacy endpoint in the Phase III study. While the CAS endpoint considers multiple clinical parameters including inflammation driven symptoms, PR is based solely on the change in proptosis. Because the degree of inflammation is decreased in chronic inactive TED, the use of CAS as opposed to PR would result in a more heterogeneous response, thus less representative of the treatment effect relevant to the need in this population.

Furthermore, PR has been consistently used as a primary efficacy endpoint in other clinical trials that investigated the efficacy and safety of treatments in patients with moderate-to-severe TED in active, chronic inactive, as well as mixed population studies.

Overall, the Sponsor considers PR as the currently best assessment to reliably assess efficacy in the target patient population composed of patients with active and chronic inactive TED.

### 3.2.4 Rationale for Biomarker Assessments

The study will assess whether biomarkers can aid in characterizing the mechanism of action of satralizumab in TED, provide evidence of pharmacodynamic activity of satralizumab in TED, or increase the knowledge and understanding of TED disease biology. Exploratory biomarker samples will be used for research purposes to identify pathway and/or disease biomarkers including but not limited to those reflective of inflammation. PD biomarker samples will be collected for the assessment of target engagement (e.g., IL-6 and soluble IL-6R [sIL-6R]) in response to satralizumab treatment.

### 3.3 Justification for Dose and Schedule

A weight-tiered dosing approach will be used in this study for the investigation of the safety and efficacy of satralizumab for the treatment of TED as shown in Table 4.

TABLE 4
Dosing Regimen for Investigation in the Phase III
Study of Satralizumab for the Treatment of TED

| Bodyweight | | |
|---|---|---|
| at Baseline | | |
| (Day 1) | | Dose, Route and Regimen |
| <40 | kg | 60 mg on Day 1 and at Weeks 2 and 4 of active |
| | | treatment initiation and then Q4W at Weeks 8, |
| | | 12, 16, and 20 administered as an SC injection |
| ≥40-≤100 | kg | 120 mg on Day 1 and at Weeks 2 and 4 of active |
| | | treatment initiation and then Q4W at Weeks 8, |
| | | 12, 16, and 20 administered as an SC injection |
| >100 | kg | 180 mg on Day 1 and at Weeks 2 and 4 of active |
| | | treatment initiation and then Q4W at Weeks 8, |
| | | 12, 16, and 20 administered as two SC injections |

Q4W = every 4 weeks; TED = thyroid eye disease.

This dosing regimen is based on a combination of sources of information, including pharmacokinetic, pharmacodynamic, efficacy, and safety data for satralizumab in the NMOSD indication. The totality of these data supports dosing regimens designed to target high levels of receptor occupancy (RO) in the population with TED. The body weight-tiered dosing proposal is intended to target exposures associated with a high RO (>90%) as demonstrated by target engagement PD biomarkers (sIL-6R) and IL-6 across the anticipated bodyweight range expected in the population with TED.

As expected for a monoclonal antibody, a clear negative correlation was seen in the Phase III studies in NMOSD between bodyweight and satralizumab exposure. Despite an approximate 3-fold range in exposure between the lightest and heaviest tertiles of participants enrolled, a high level of predicted RO (median RO_{tr,ss}>95%) was maintained throughout the dosing interval. Although it is therefore not possible to define the target RO_{rs,ss} value associated with efficacy, similar clinically meaningful efficacy was demonstrated in all bodyweight quartiles. The recommended dosing regimen for the population with NMOSD is therefore a fixed 120 mg dose for participants of all body weights.

The rationale for the weight-based tiered dosing regimen in this study is provided in the following sections.

### 3.3.1 Dosing Regimen and Target Engagement in Participants >100 kg

The few patients with NMOSD in whom predicted RO., values <80% were noted, generally had baseline (Day 1) bodyweights >100 kg.

While review of this group of participants did not indicate any apparent lack of efficacy in this group (data available on request), the number of participants was small (10 participants receiving satralizumab during the double-masked period, approximately 10% of the population enrolled globally, as compared with approximately 20% in the wider U.S. population [real-world data available on request]). Therefore, this study will investigate higher doses in this bodyweight range in order to maximize the blockade of IL-6R across the dosing interval, and therefore optimize the potential for efficacy in the population with TED.

Early masked review of PK data in the Phase III study in generalized myasthenia gravis has confirmed that the 120 mg<100 kg >180 mg regimen better achieves the target range of exposure and RO (defined as the predicted ranges for the 120 mg<100 kg>180 mg in NMOSD) than a higher dosing regimen (180 mg<100 kg>240 mg), indicating that the pharmacokinetics of satralizumab in these two patient populations are more similar to each other than to the healthy volunteer population. Building on this understanding, the pharmacokinetics of satralizumab in TED is therefore assumed to be similar to that in NMOSD, supporting the use of the same 120 mg<100 kg>180 mg regimen in TED. In addition, the evidence from a Roche analysis using U.S. electronic health record data (available on request) indicates that the median and range of bodyweights (known to correlate inversely with exposure for monoclonal antibodies) for the population with TED are similar to those for the population with NMOSD, including a similar percentage of participants with bodyweight >100 kg. The simulations that support the appropriateness of this tiered dosing regimen, performed using the existing population PK (pop-PK) model (based on data in healthy volunteers and NMOSD, Report No. 1094498) accounting for constant covariates known to impact the pharmacokinetics of satralizumab, are presented in the following sections.

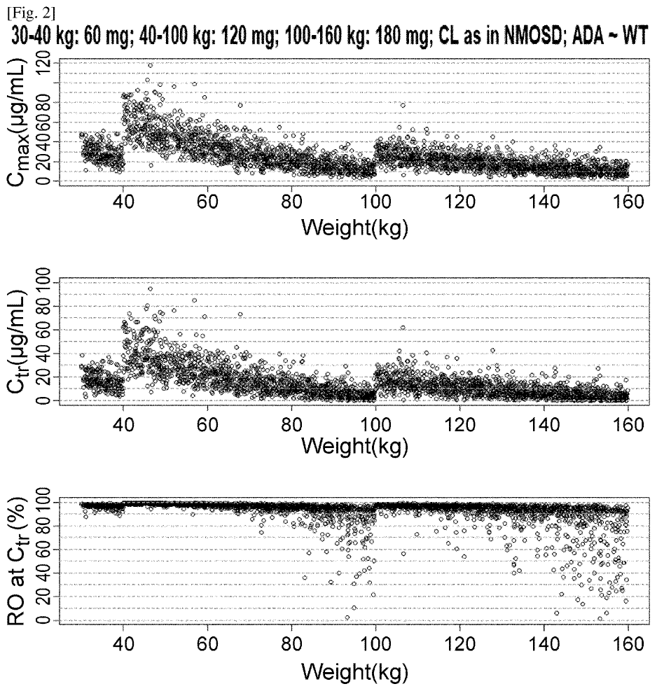

The predicted maximum concentration (Cₘₐₓ), steady-state concentration at the end of a dosing interval (C_{trough}), and RO values for participants of bodyweight range 30-160 kg based on the bodyweight-tiered dosing scheme are shown in FIG. 2. These suggest that for participants for bodyweight >100 kg, a regimen of 180 mg Q4W would be associated with median C_{max,ss} values and Ctr,ss values that are similar to those achieved in participants of between 40 and 100 kg, shown to be associated with near maximal RO throughout the dosing interval. Significantly, the range of predicted exposures in participants >100 kg receiving 180 mg remains lower than the maximum achieved using 120 mg Q4W in participants of low bodyweight (40-50 kg) in the Phase III trials in NMOSD, and therefore exposure-safety coverage is preserved.

### 3.3.2 Dosing Regimen and Target Engagement in Participants <40 kg

Conversely, the median exposures achieved in the lightest tertile of patients with NMOSD are in excess of those required to maintain near maximal target engagement over the dosing interval, although this was not associated with a worse safety profile than in heavier participants. This is consistent with the fact that once exposures leading to full inhibition of the IL-6 signaling pathway (i.e., full, sustained RO) have been reached, further impact on white blood cells and neutrophils (directly connected with the inhibition of the IL-6 signaling pathway and thus expected as a result of satralizumab treatment), is not expected. Thus, the fixed dosing regimen is associated with a positive benefit-risk profile in all bodyweight groups.

Despite the comparable safety profile for satralizumab across the bodyweight range, the Sponsor is mindful of targeting only those exposures needed to achieve high RO. Therefore, an additional (lower) dosing tier (60 mg, see FIG. 2) will be used for investigation in participants in this range, supported by simulation using the available pop-PK model.

The simulations presented indicate that exposures achieved using this lower dose will be sufficient to maintain near-maximal RO over the dosing interval in most participants. In addition, given that this lower regimen for participants <40 kg represents a 50% reduction from the recommended 120 mg dose, the predicted exposures are well within the observed exposures in NMOSD, providing adequate safety coverage.

### 3.4 End of Study Definition

A participant is considered to have completed the study if he or she has completed all parts of the study, including the Part I (Day 1-Week 24), Part II (Weeks 24-48) and follow-up period (Weeks 48-72).

The end of this study is defined as the date of the last visit of the last participant in the study or the date at which the last data point required for statistical analysis or safety follow-up (weeks 48-72) is received from the last participant, whichever occurs later. The end of the study is expected to occur approximately 72 weeks after the last participant is enrolled.

In addition, the Sponsor may decide to terminate the study at any time.

### 3.5 Duration of Participation

The total duration of study participation for each individual is expected to be approximately 72 weeks.

### 4. Study Population

Approximately 120 adult (18 years or older) male and female patients with moderate-to-severe active and chronic inactive TED will be enrolled in this study at up to 40 study sites globally.

Prospective approval of protocol deviations to recruitment and enrollment criteria, also known as protocol waivers or exemptions, is not permitted.

### 4.1 Inclusion Criteria

Inclusion Criteria for Individuals with Moderate-to-Severe Active TED
Clinical diagnosis of active TED with 3 or more of CAS (on the 7-item scale) at screening and baseline (Day 1) in the study eye.
Diagnosis of active moderate-to-severe TED; usually associated with proptosis (exophthalmos) 3 mm or more above normal for race and gender in the study eye.

Additionally, participants should have one or more of the following: 2 mm or more lid retraction, moderate or severe soft tissue involvement, and/or inconstant or constant diplopia.
Onset of active TED symptoms in the study eye (as determined by participant medical records) within 12 months prior to baseline (Day 1).
Euthyroid with the baseline disease under control, or have mild hypo- or hyperthyroidism (defined as free T3 and free T4<50% above/below normal limits at screening).
   Inclusion Criteria for Individuals with Chronic Inactive TED
Clinical diagnosis of stable, chronic (inactive) TED, as determined by participant medical records indicating CAS<3 (on a 7-item scale) in both eyes for at least 6 months prior to screening, or all of the following:
   No progression in proptosis for at least 6 months prior to screening
   If participant has a history of diplopia due to TED, no progression in diplopia for at least 6 months prior to screening
   No new inflammatory TED symptoms for at least 6 months prior to screening.
CAS <3 (on a 7-item scale) in both eyes at screening and baseline (Day 1) visits
Initial TED diagnosis >12 months but <10 years prior to screening
History and presence at screening and baseline of chronic TED as estimated by treating physician with proptosis (exophthalmos) 3 mm or more above normal for race and gender in the study eye. Additionally, participants should have one or more of the following: 2 mm or more lid retraction, moderate or severe soft tissue involvement, and/or inconstant or constant diplopia.
Euthyroid with the background disease under control, or have mild hypo- or hyperthyroidism (defined as free T3 and free T4<50% above/below normal limits at screening).

### 4.2 Exclusion Criteria

Potential participants are excluded from the study if any of the following criteria apply:
Decrease in CAS of 2 points or more in the study eye between Screening and Study Baseline (Day 1)
Decrease in proptosis of 2 mm or more in the study eye between Screening and Study Baseline (Day 1)
Decreased best corrected visual acuity (BCVA) due to dysthyroid optic neuropathy as defined by a decrease in vision of two lines on the Snellen chart, new visual field defect, or color defect secondary to optic nerve involvement within the last 6 months
Requiring immediate or urgent medical treatment with CSs for TED, in the judgment of the investigator
Identified pre-existing ophthalmic disease that, in the judgment of the investigator, would preclude study participation or complicate interpretation of study results, including corneal decompensation unresponsive to medical management and including ophthalmic diseases that will likely require prohibited therapy during the study (e.g., uveitis, idiopathic orbital inflammatory disease)
Requiring immediate surgical ophthalmological intervention or planning corrective surgery or irradiation during the course of the study, in the judgment of the investigator
Requiring radioiodine therapy for normalization of thyroid function in the course of the study
Sight-threatening TED characterized by dysthyroid optic neuropathy and/or corneal ulceration (defined as a deep corneal erosion which extends through epithelium and Bowman's membrane into corneal stroma)
Pregnancy or breastfeeding, or intention of becoming pregnant during the study or within 12 weeks after the final dose of satralizumab.

Female participants of childbearing potential must have a negative serum pregnancy test result at screening and negative urine dipstick pregnancy test prior to initiation of study treatment.
Any surgical procedure within 4 weeks prior to baseline (Day 1) Minor surgeries defined as procedures requiring only local anesthesia or conscious sedation and are done on an ambulatory or outpatient basis (e.g., toenail surgery, mole surgical excision, tooth extraction) are allowed.
Plans to have surgical procedure (except minor surgeries) during the study
Active or presence of recurrent bacterial, viral, fungal, mycobacterial infection, or other infection (excluding fungal infection of nail beds or dental caries) at baseline (Day 1)
Infection requiring treatment with IV anti-infective agents within 4 weeks prior to baseline (Day 1)
Evidence of progressive multifocal leukoencephalopathy
Congenital or acquired immunodeficiency, including HIV infection
Positive hepatitis B (HBV) test at screening (defined as either of the following):
Positive hepatitis B surface antigen (HBsAg)
Positive total hepatitis B core antibody (total HBcAb) confirmed by a positive viral DNA polymerase chain reaction test
Positive hepatitis C (HCV) test at screening (defined as positive HCV antibody and detectable HCV RNA) Participants with positive HCV antibody and undetectable HCV RNA 12 weeks after HCV treatment completion are eligible to participate in the study.
Evidence of latent or active tuberculosis (TB), excluding participants receiving chemoprophylaxis for latent TB infection
If a participant is positive for latent TB, then the participant must be treated with appropriate anti-mycobacterial therapy for at least 4 weeks prior to initiating study treatment administration
Receipt of live or live attenuated vaccine within 6 weeks prior to baseline (Day 1)
History of diverticulitis or concurrent severe gastrointestinal (GI) disorders (such as symptomatic diverticulosis) that, in the investigator's opinion, may lead to increased risk of complications such as GI perforation
History of blood donation (one unit or more), plasma donation or platelet donation within 90 days prior to screening
History of malignancy within the last 5 years prior to baseline (Day 1), including solid tumors, hematologic malignancies and in situ carcinoma (except basal cell and squamous cell carcinomas of the skin, or in situ carcinoma of the cervix uteri that have been completely excised and cured)
History of severe allergic reaction to a biologic agent (e.g., shock, anaphylactic reactions)
History of drug or alcohol abuse within 1 year prior to baseline (Day 1)
Any serious medical condition or abnormality in clinical laboratory tests that, in the investigator's judgment, precludes an individual's safe participation in and completion of the study
Presence of any of the following laboratory abnormalities at screening:
   WBC <3.0×10³/micro L
   ANC <2.0×10³/micro L
   Absolute lymphocyte count <0.5×10³/micro L
   Platelet count <10×10⁴/micro L
   AST or ALT >1.5× upper limit of normal (ULN)

If a laboratory retest is conducted, the last retest value before baseline (Day 1) must meet study eligibility criteria.

Exclusion Criteria for all Individuals with TED. Both Active and Inactive:
Previous orbital radiotherapy in the study eye
Previous unilateral or bilateral orbital decompression surgery
Previous exposure to biologic drugs directly targeting IL-6 or IL-6 receptor (including, but not limited to, tocilizumab)
Use of any investigational agent for any condition within 60 days prior to screening or anticipated use during the course of the trial.
Prior strabismus surgery in either eye
   Exclusion Criteria for Individuals with Active TED Only:
Any previous use of systemic, non-biologic, non-steroidal immunosuppressive agents for the treatment of TED (e.g., methotrexate, mycophenolate or cyclosporine)
Previous treatment with any other systemically administered immunomodulatory biological agents for TED, including but not limited to rituximab, teprotumumab, secukinumab, batoclimab at any time prior to screening
Any prior CS use (IV or oral) with a cumulative dose equivalent to 1 g or more of methylprednisolone or equivalent for the treatment of TED.
Any CS use (periocular, IVT [intravitreal] injections or implants, IV or oral) for conditions other than TED within 4 weeks of screening Previous CSs use (IV or oral) with a cumulative dose of <1 g methylprednisolone or equivalent for the treatment of TED, IVT injections or implants is allowed if discontinued at least 4 weeks prior to screening.

Previous CS eyedrop use is allowed if discontinued at least 7 days prior to screening. Topical CSs for dermatological conditions are allowed at any time prior to, or during the study

Exclusion Criteria for Individuals with Inactive TED Only:
Use of systemic, non-biologic, non-steroidal immunosuppressive agents for TED within 3 months prior to screening
Treatment with any systemically administered immunomodulatory biological agent for TED within 6 months prior to screening.
Use of any CSs (periocular, IV, oral, IVT injections or implants) for any indication within 3 weeks prior to screening.

Previous CS eyedrop use within 7 days prior to screening.

Topical CSs for dermatological conditions are allowed at any time prior to, or during the study.

### 5. Study Treatment, Other Treatments Relevant to the Study Design, and Concomitant Therapy

The investigational medicinal products (IMP) for this study are satralizumab (RO5333787) and placebo for satralizumab.

IMP will be supplied by the Sponsor as prefilled syringe (PFS) assembled with a plunger rod and needle safety device (NSD) filled with 1.0 mL of solution for SC injection, corresponding to 120 mg satralizumab. Satralizumab placebo PFS is identical in composition to satralizumab PFS, but does not contain the satralizumab active ingredient and will be identical in appearance and packaging to satralizumab.

For the 60 and 180 mg doses, a separate dose of 60 mg will be prepared using the ‘empty vial method’. In this method, the 120 mg PFS containing 1 mL of solution will be transferred into an empty vial, and half the volume (0.5 mL, corresponding to 60 mg dose) will be withdrawn into the application syringe. For more details, please refer to the pharmacy manual.

### 5.1 Study Treatments Administered

### 5.1.1 Satralizumab and Placebo

Satralizumab or placebo will be administered by SC injection in the abdominal or femoral region by the investigator or designated person after all other study-related procedures have been performed at a site visit.

In the Part I period, participants will receive satralizumab or placebo at Day 1 and Weeks 2 and 4 (loading doses) and then Q4W at Weeks 8, 12, 16 and 20 (maintenance doses) (seven doses in total). The dose of study treatment will be determined based on participant's body weight. Participants will receive one or two injection(s) of satralizumab according to body weight at 60 mg (<40 kg), 120 mg (between 40 and 100 kg), or 180 mg (>100 kg). The initial dose of study treatment will be determined as described for Part I of the study and will be based on the body weight at baseline (Day 1). The dose may be adjusted based on changes in body weight during the study.

The treatment which participants receive in Part II of the study is determined by their PR at the end of Part I (Week 24 primary endpoint):
Participants who are proptosis non-responders at Week 24 will receive SC satralizumab treatment during Part II (Weeks 24-44).
Participants who are proptosis responders at Week 24 will be re-randomized in a 1:1 ratio to receive SC satralizumab or matching placebo (Weeks 24-44), regardless of their original treatment assignment in Part I.

Due to the study design, participants who received placebo during Part I and receive satralizumab in Part II will receive a loading dose of satralizumab at Week 26; participants who received satralizumab in Part I and receive satralizumab in Part II do not require a loading dose and will receive placebo at Week 26.

Study drug will be administered within a +/−3-day window at Weeks 2 and 4 for Part I, and Weeks 26 and 28 for Part II; and within a +/−7-day window for all other dosing visits. The minimal dosing interval should be 8 days for loading doses and 14 days for maintenance doses.

For the first five doses of study drug in Part I and Part II of the study, all participants should be observed for the signs and symptoms of hypersensitivity reactions at the study site for at least 1 hour after administration of study treatment.

### 5.2 Concomitant Therapy

Any medication (including over-the-counter or prescription medicines, nutritional supplements, homeopathic and/or herbal remedies or vaccine) used by a participant in addition to protocol-mandated treatment from 7 days prior to initiation of study treatment to the final study visit (Week 72 visit, or early discontinuation visit) must be recorded on the Concomitant Medications eCRF(s) along with the following information:
Reason for use
Dates of administration, including start and end dates
Dosage information, including dose and frequency

The Medical Monitor may be consulted if there are any questions related to concomitant or prior therapy.

### 5.2.1 Permitted Therapy

In general, investigators may manage a participant's care (including preexisting conditions) through use of supportive therapies, as clinically indicated and per local standard practice.

Use of the following concomitant therapies is permitted as described below:
Anti-thyroid medications such as methimazole, carbimazole and propylthiouracil
Thyroid replacement therapy such as levothyroxine
Local supportive measures for TED including simple analgesics (e.g., acetaminophen, non-steroidal anti-inflammatory therapies), and ocular lubricants
Medications/supplements for conditions other than TED
Oral contraceptives (see Section 4.1)

### 6. Study Assessments and Procedures

### 6.1 Efficacy Assessments

### 6.1.1 Standard Ocular Assessments

Ocular assessments will be performed for both eyes, unless otherwise indicated. The following assessments must be performed, in the same order:
Refraction and BCVA assessed on Snellen visual acuity chart at a starting test distance of 6 meters/20 feet (perform prior to all predose assessments)
Eyelid measurement of both eyes including margin reflex distance (MRD) 1, MRD2, interpalpebral fissure distance, lagophthalmos, and superior and inferior scleral show (perform prior to all predose assessments)
Iris pigmentation assessment (light and dark grading)
Intraocular pressure measurement
Slit-lamp examination Undilated anterior segment evaluation will include corneal staining with fluorescein using the standardize Oxford grading system
Dilated binocular indirect high magnification ophthalmoscopy or dilated examination of posterior segment on a slit-lamp with a high power lens

This will include detailed posterior segment evaluation with focus on determining presence/absence of optic neuropathy and choroidal folds.

6.1.2 Ocular Assessments Specify to Thyroid Eye Disease

### 6.1.2.1 Proptosis (Exophthalmos)

Proptosis assessments will be performed on the “study eye” (defined as the most severely affected as compared with the fellow eye) first, followed by the other eye. All assessments will be performed using Sponsor provided Hertel exophthalmometer to maintain consistency in measurement. The same Hertel instrument and same observer should be used at each evaluation (except when strictly unavoidable) for the entire duration of the study. In addition, the same intercanthal distance or base setting must be used on each occasion.

Proptosis will be measured at screening and baseline (Day 1), and then at 4-week intervals till the final visit (48 weeks). Individuals who have a 2 mm or more decrease in proptosis from screening to baseline in the study eye are not eligible for randomization. Measurements will be recorded on the Clinical Measures of Severity eCRF under exophthalmos. The baseline value will also be recorded.

### 6.1.2.2 Diplopia

Diplopia grade will be assessed with the use of the Gorman subjective diplopia score (range: 0 to 3 points), which includes: no diplopia (absent, scored as 0), diplopia in the primary position of gaze when the patient is tired or awakening (intermittent, scored as 1), diplopia at extremes of gaze (inconstant, scored as 2), and continuous diplopia in the primary or reading position (constant, scored as 3). Measurements will be recorded on the Clinical Measures of Severity eCRF under diplopia (Bartalena et al. 2008).

### 6.1.2.3 Motility Restriction

Motility is examiner assessed by estimating the degrees of restriction in eye movements. It will be assessed at the same timepoints as the Clinical Measures of Severity.

Monocular excursions in horizontal and vertical directions of gaze are recorded using the light reflex test [Dolman et al, 2012].

The clinician will shine a pen light in line with the eye being examined in ambient room light and observe the participant's eye along the light's axis. The participant will be asked to look in the four cardinal directions and the position of the light reflex is viewed on the surface of the cornea. If the light touches the limbus, the eye is assessed to be turned 45 degrees; if half way between the limbus and pupil edge, the eye is assessed at 30 degrees; and if it is at the pupil edge, it is assessed at 15 degrees. Intermediate ductions are judged by estimating the light position between these points to the nearest 5 degrees.

The monocular ductions of each eye (degrees) will be recorded for adduction, abduction, supraduction and infraduction.

### 6.1.2.4 Clinical Activity Score

The CAS will be completed at screening, baseline (Day 1) and Weeks 4, 12, 20, and 24 during the Part 1 period, and at Weeks 28, 36, 44 and 48 during the Part II period using the 7 item EUGOGO amended CAS (Wiersinga et al., 2006).

The CAS must be 3 or more points for enrollment and randomization of patients with active TED and CAS must be <3 points for enrollment and randomization of patients with chronic inactive TED. Individuals whose CAS score decreases 2 or more points from screening to baseline are not eligible for randomization.

TABLE 5
Clinical Activity Score Assessment

| Item^{a} | Description |
|---|---|
| 1. | Spontaneous orbital pain. |
| 2. | Gaze evoked orbital pain. |
| 3. | Eyelid swelling that is considered to be due to TED. |
| 4. | Eyelid erythema. |
| 5. | Conjunctival redness that is considered to be due to |
| | TED (ignore “equivocal” redness). |
| 6. | Chemosis. |
| 7. | Swelling of caruncle or plica. |

TED = thyroid eye disease.
^{a}Each item is scored (1 = present; 0 = absent) and scores for each item are summed for total score.

To promote consistency in data collection across clinical trial sites, all investigators will be provided with training. Except when strictly unavoidable, the same observer should conduct each CAS evaluation for the full duration of the study.

### 6.1.2.5 Clinical Measures of Severity

The severity of the disease will be evaluated using the EUGOGO guidelines (Bartalena et al, 2008). The following items will be assessed at screening, baseline (Day 1) and Weeks 4, 12, 20, and 24 during the Part I period, and Weeks 28, 36, 44, and 48 during the Part II period. Except when strictly unavoidable, the same observer should conduct each evaluation of severity measure for the full duration of the study

EUGOGO (Bartalena, et al., 2021) recommends the following classification of patients with TED:
Mild TED: Patients usually present with one or more of the following characteristics: lid retraction <2 mm, mild soft tissue involvement, exophthalmos <3 mm over the normal for the race or gender, transient or no diplopia and corneal exposure responsive to lubricants.
Moderate-to-severe TED: Patients usually present two or more of the following characteristics: 2 mm or more eyelid retraction, moderate to severe soft tissue involvement, exophthalmos at least 3 mm more than normal for their race or gender, and intermittent or constant diplopia.
TED that is sight-threatening or severe: Patients usually present with optical neuropathy and/or cornea deterioration.

### 6.1.2.6 Corneal Staining

After instillation of 2% sodium fluorescein in the study eye, fluorescein staining of the cornea will be observed using the cobalt blue filter on the slit-lamp biomicroscope. Corneal staining will be graded using the Oxford Grading Scheme Chart (a 6-point scale that ranges from Grade 0 to V), with each investigator using the same set of image provided by the Sponsor as a guide at baseline (Day 1) and Weeks 4, 12, 20, and 24 during the Part I period, and Weeks 28, 36, 44 and 48 during the Part II period. Staining assessment will be based on the intensity of fluorescein staining, ranging from Grade 0 to V for each panel (Grade 0-I: normal; Grade II-III: mild to moderate; Grade IV-V: severe).

### 6.1.3 Clinical Outcome Assessments

PRO and clinician-reported outcome (ClinRO) instruments will be completed to assess the treatment benefit of satralizumab. In addition, PRO instruments will enable the capture of each participant's direct experience with satralizumab. PRO data will be collected through use of the GO-QoL. ClinRO data will be collected through the use of OSDI.

6.1.3.1 Grave's Ophthalmopathy Quality of life Questionnaire

The GO-QoL questionnaire (Terwee et al, 1998) will be completed at baseline, Weeks 12 and 24 during the Part I period, and at Weeks 36 and 48 during the Part II period.

The GO-QoL is a 16-item self-administered questionnaire divided into two subscales and used to assess the perceived effects of TED by the participants on their: 1) Visual Functioning; and 2) Appearance.

### 6.1.3.2 Ocular Surface Disease Index

The OSDI instrument is a validated dry eye questionnaire, designed to be administered by health professionals to participants. This instrument will assess cornea and ocular surface symptoms for each participant. The OSDI questionnaire (Schiffman et al, 2000) will be completed at baseline (Day 1), Weeks 12 and 24 during the Part I period, and at Weeks 36 and 48 during the Part II period. The OSDI questionnaire consists of three main sections concerning ocular symptoms, visual function, and environmental factors. The OSDI score is a composite measure built on 12 questions, with totals ranging from 0 to 100, and higher scores representing a worse disease index.

### 6.2 Additional Assessments and Procedures Requiring Separate Consent or Performed Only at Participating Sites

### 6.2.1 Optional Imaging

Collection and submission of optional imaging is contingent upon review and approval of the imaging portion of the Informed Consent Form by each site's IRB/EC and, if applicable, an appropriate regulatory body. If a site has not been granted approval for imaging, this section of the protocol will not be applicable at that site. The central reading center (CRC) will provide sites with the CRC manual and training materials for imaging. All images will be obtained by trained site personnel at the study sites and forwarded to the CRC for independent analysis and/or storage.

### 6.2.1.1 Optional Orbital Imaging

For participants who consent to have orbital magnetic resonance imaging, imaging will be taken by trained site personnel at the study sites and forwarded to the CRC for possible evaluation and/or storage, and will later be transferred to Roche. The imaging should be performed prior to satralizumab or placebo treatment at baseline (Day 1) and Weeks 24 and 48. Extraocular muscle and orbital fat volumes will be measured by means of three-dimensional volumetric quantification.

Note: After randomization, if a patient misses a study visit when orbital imaging are scheduled or the images are not taken at the scheduled visit (e.g., due to broken equipment), they should be obtained at the next scheduled visit the patient attends.

### 6.2.1.2 Optional Facial Photography

Optional facial photographs will be collected for participants who consented at selected study sites or at sites per investigator discretion that have this imaging capability. All images will be forwarded to the CRC for possible evaluation and/or storage, and will later be transferred to Roche. Photographs should be obtained prior to satralizumab or placebo treatment at baseline (Day 1) and Weeks 24 and 48.

Note: After randomization, if a patient misses a study visit when facial photographs are scheduled or the photos are not taken at the scheduled visit (e.g., due to broken equipment), they should be obtained at the next scheduled visit the patient attends.

Additional non-protocol clinical assessments or imaging procedures may be performed as part of standard of care by the investigator at the physician's discretion at the scheduled or unscheduled safety visits (e.g., visual field, color vision tests with color plates or red desaturation, computer tomography), but these do not need to be submitted to the CRC. The Sponsor will not reimburse the site for these non-protocol clinical assessments or imaging procedures beyond the reimbursable per protocol.

### 7. Statistical Considerations

The primary analysis will be performed when all participants have either completed the study through Week 24 or have discontinued from the study prior to Week 24, whichever comes later. An update analysis will be performed when all participants have either completed the study through Week 48 or have discontinued from the study prior to Week 48; whichever comes later.

The final analysis database lock will be performed when all participants have either completed the study through Week 72 (last participant last visit) or have discontinued early from the study, whichever comes later.

The efficacy populations are defined as follows:
Active full analysis set (FAS): All participants who are randomized in the study with active disease as defined by the inclusion and exclusion criteria. For analyses based on this patient population, participants will be grouped according to the treatment assigned at randomization.
Overall FAS: All participants who are randomized in the study. For analyses based on this patient population, participants will be grouped according to the treatment assigned at randomization.

### 7.1 Statistical Hypotheses

The primary efficacy endpoint is investigator-assessed PR, defined as the proportion of participants achieving a 2 mm or more reduction in proptosis from baseline (Day 1) to Week 24 in the study eye, provided there is no corresponding deterioration of proptosis (2 mm or more increase) in fellow eye in the active FAS. The following hypothesis will be tested:
Superiority of satralizumab Q4W versus placebo in PR in the active FAS

The null and alternative hypothesis for the superiority test are as follows:
The null hypothesis (He) is:

$${P}_{satralizumab} - {P}_{placebo} = 0$$

The alternative hypothesis (H₀) is:

$${P}_{satralizumab} - {P}_{placebo} \neq 0$$

where P_{satralizumab} and P_{placebo} are the expected PR for the Satralizumab arm and placebo arm, respectively.

If the observed rate is higher in the treatment arm and the two-sided p-value <0.05, the test is considered positive and satralizumab is considered superior to placebo.

A fixed-sequencing test approach will be used to control the overall type I error rate for the hypothesis testing of the primary and key secondary endpoints.

If the primary endpoint test is positive, superiority of satralizumab compared with placebo for the following key secondary endpoints will be tested at the two-sided 5% significance level in the order as shown below:
Proportion of participants achieving proptosis response from baseline (Day 1) at Week 24 in the overall FAS
Change in proptosis from baseline (Day 1) to Week 24 in the active FAS
Change in proptosis from baseline (Day 1) to Week 24 in the overall FAS
Proportion of participants with Grade 1 or more reduction/improvement in diplopia from baseline (Day 1) at Week 24 in the active FAS among participants with diplopia present at baseline
Proportion of participants with Grade 1 or more reduction/improvement in diplopia from baseline (Day 1) at Week 24 in the overall FAS among participants with diplopia present at baseline
Proportion of participants achieving absence of motility-induced pain from baseline (Day 1) at Week 24 in the active FAS among participants with motility-induced pain present at baseline
Proportion of participants achieving absence of spontaneous pain from baseline (Day 1) at Week 24 in the active FAS among participants with spontaneous pain present at baseline

For hypotheses that use binary endpoints, the corresponding estimand framework will follow the primary estimand framework.

The continuous key secondary endpoints will be tested using the mixed-effect model for repeated measures method. The respective estimand framework can be found in Table 1.

### 7.1.1 Sample Size Determination

A total of 120 participants including 25% chronic inactive participants will be enrolled in this study and randomized in a 1:1 ratio.

A total of 90 participants with active TED will provide >90% power to show superiority of satralizumab compared with placebo in PR at Week 24. The following assumptions are used:
Response ratio of 0.6 in the satralizumab arm
Response ratio of 0.2 in the placebo arm
Fisher exact test for two proportions
5% two-sided type I error rate
10% dropout rate

While the Cochran-Mantel-Haenszel (CMH) test will be used as the main analysis method, the Fisher exact test is expected to be less sensitive and a more conservative way to estimate the sample size.

A total of 120 active and chronic inactive participants (overall FAS) will provide >90% power to show superiority of satralizumab compared with placebo in PR at Week 24. The following assumptions are used:
Response ratio of 0.6 in the satralizumab arm
Response ratio of 0.275 in the placebo arm
Fisher exact Test for two proportions
5% two-sided type I error rate
10% dropout rate

### 7.2 Statistical Analyses

The Statistical Analysis Plan for Part I will be finalized prior to Sponsor unmasking, and it will include a more technical and detailed description of the statistical analyses described in this section. This section is a summary of the planned statistical analyses of selected endpoints, including primary and key secondary endpoints. For the analysis of Part II a separate statistical analysis plan will be written. The analyses specified in the Statistical Analysis Plan supersede those specified here.

### 7.2.1 Estimation Methods for the Primary Estimand

The primary estimand is defined in Table 1. The main analysis for the primary endpoint will be the CMH test stratified by smoking status (current smoker, including the use of e cigarettes vs. former smoker vs. never smoked), and the p-value will be provided. The proportion of participants in each treatment group and the overall difference in proportions between treatment groups will be estimated using the weighted average of the observed proportions and the differences in observed proportions over the strata defined by smoking status using the CMH weights. CIs of the proportion of participants in each treatment group will be calculated using the Wilson method, and the CI for the difference in proportions between treatment groups will be calculated using the Newcombe method. Missing values will be imputed using multiple imputation methods.

A supplementary analysis will be performed using the same intercurrent events, analysis methods and missing data handling approach as the main analysis of the primary endpoint, with the exception that participants with any intercurrent event will be treated as non-responders.

A sensitivity analysis will be performed using the same intercurrent events, handling of intercurrent events and analysis methods as the main analysis of the primary endpoint, with the exception that missing data will not be imputed and participants with missing data will be excluded from the analysis.

### 7.2.2 Analyses for the European Medicines Agency

To support registration in the European Union, a separate set of hypotheses will be tested in the active and overall population after the primary hypothesis. For the European Medicines Agency, the following hypotheses will be tested hierarchically at an overall significance level of alpha=0.05 to control the overall type I error rate:
Proportion of participants achieving overall response at Week 24 in the active FAS
Proportion of participants achieving PR at Week 24 in overall FAS
Proportion of participants achieving 2-point or more reduction in CAS from baseline (Day 1) at Week 24 in the active FAS
Change in proptosis from baseline (Day 1) to Week 24 in the active FAS
Change in proptosis from baseline (Day 1) to Week 24 in the overall FAS
Proportion of participants with a 6-point or more improvement in the Visual Functioning and Appearance sub-scale scores of the GO-QoL at Week 24 in the active FAS
Proportion of participants with a 6 point or more improvement in the Visual Functioning and Appearance sub-scale scores of the GO-QoL at Week 24 in the overall FAS
In subset of active TED participants with baseline diplopia, proportion of participants with Grade 1 or more reduction or improvement in diplopia at Week 24 in active FAS
In subset of overall (active and chronic inactive) population with baseline diplopia, proportion of participants with Grade 1 or more reduction or improvement in diplopia at Week 24 in overall FAS Intercurrent events will be defined as in the primary estimand definition. A treatment policy approach will be used for handling all intercurrent events.

### 7.2.3 Analyses for the Japan Pharmaceuticals and Medical Devices Agency

To support registration in Japan, a separate set of hypotheses will be tested in the active and overall population after the primary hypothesis. For the Pharmaceuticals and Medical Devices Agency, the following hypotheses will be tested hierarchically at an overall level of alpha=0.05 to control the overall type I error rate:
Proportion of participants achieving PR at Week 24 in the overall FAS
Proportion of participants achieving overall response at Week 24 in the active FAS
Change in proptosis from baseline (Day 1) to Week 24 in the active FAS
Proportion of participants achieving 2-point or more reduction in CAS from baseline (Day 1) at Week 24 in the active FAS
Change in proptosis from baseline (Day 1) to Week 24 in the overall FAS
Proportion of participants with a 6-point or more improvement in the Visual Functioning and Appearance sub-scale scores of the GO-QoL at Week 24 in the active FAS
Proportion of participants with a 6 point or more improvement in the Visual Functioning and Appearance sub-scale scores of the GO-QoL at Week 24 in the overall FAS
In subset of active TED participants with baseline diplopia, proportion of participants with Grade 1 or more reduction or improvement in diplopia at Week 24 in active FAS
In subset of overall (active and chronic inactive) population with baseline diplopia, proportion of participants with Grade 1 or more reduction or improvement in diplopia at Week 24 in overall FAS

### 7.2.4.3 Pharmacokinetic Analyses

The PK analysis population consists of all participants in the safety analysis set with at least one valid post-dose concentration result with a dosing record and sampling time. The trial will evaluate the PK characteristics of satralizumab treatment over 24 weeks in Part I of the study by summary statistics. PK samples will also be taken during Part II of the study, to further characterize the pharmacokinetics of satralizumab in TED over longer-term treatment. The serum concentration at each sampling timepoint will be described with means and standard deviation of C_{trought} irrespective of whether participants receive rescue therapy, miss a dose, or if study drug administration is delayed, or if they withdraw from treatment before data collection at Week 24. Individual and mean serum-concentration-versus-time curves will be plotted. Nonlinear mixed effects analysis will be performed to analyze the satralizumab concentration-time data collected in the trial. The model to be used was previously developed on the basis of PK data from adult healthy volunteers and adult and adolescent participants with NMOSD. Further model development may be undertaken if needed in order to achieve a satisfactory description of the data, and the data from this study may be pooled with data from other studies with satralizumab. Population and individual PK and exposure parameters will be generated based on the model. Covariate analysis, including demographic factors and ADA status, will also be performed. Both the satralizumab concentration data and the results of the pop PK analysis will be reported separately from the CSR.

### 7.2.4.4 Immunogenicity Analyses

The immunogenicity analysis population will consist of all participants with at least one ADA assessment. Participants will be grouped according to treatment received or, if no treatment is received prior to study discontinuation, according to treatment assigned.

The number and proportion of ADA-positive participants and ADA-negative participants at baseline (baseline prevalence) and after drug administration (postbaseline incidence) will be summarized by treatment group. When determining postbaseline incidence, participants are considered to be ADA positive if they are ADA negative or have missing data at baseline but develop an ADA response following study drug exposure (treatment-induced ADA response), or if they are ADA positive at baseline and the titer of one or more postbaseline samples is at least 0.60-titer unit greater than the titer of the baseline sample (treatment-enhanced ADA response). Participants are considered to be ADA negative if they are ADA negative or have missing data at baseline and all postbaseline samples are negative, or if they are ADA positive at baseline but do not have any postbaseline samples with a titer that is at least 0.60 titer unit greater than the titer of the baseline sample (treatment unaffected).

The percentage of participants who have positive or negative ADA results for satralizumab will be tabulated. PK, PD, efficacy parameters, and safety will be summarized by anti-satralizumab antibody (i.e., satralizumab ADA) status.

### Pharmacodynamic Analyses

Serum IL-6 and sIL-6R levels will be summarized by treatment group and timepoint graphically and descriptively, as appropriate.

ABBREVIATIONS

| Abbreviation | |
|---|---|
| or Term | Definition |
| ADA | anti-drug antibody |
| AUC | area under the concentration-time curve |
| BCVA | best corrected visual acuity |
| Cₘₐₓ | maximum concentration |
| C_{trough} | steady-state concentration at the end of the dosing interval |
| CAS | Clinical Activity Score |
| CMH | Cochran-Mantel-Haenszel (test) |
| CRC | central reading center |
| CS | corticosteroids |
| CSR | Clinical Study Report |
| EC | Ethics Committee |
| eCRF | electronic Case Report Form |
| EUGOGO | European Group on Grave's Orbitopathy |
| FAS | full analysis set |
| GO | Grave's ophthalmopathy/orbitopathy |
| GO-QoL | Grave's orbitopathy- Quality of Life |
| HBV | hepatitis B |
| HBsAg | hepatitis B surface antigen |
| ICH | International Council for Harmonisation |
| iDCC | independent Data Coordinating Center |
| iDMC | independent Data Monitoring Committee |
| IL | interleukin |
| IL-6R | interleukin-6 receptor |
| IMP | investigational medicinal product |
| IRB | Institutional Review Board |
| IST | immunosuppressant |
| IVT | intravitreal |
| IxRS | interactive voice or web-based response system |
| MRD | margin reflex distance |
| NCI CTCAE | National Cancer Institute Common Terminology Criteria |
| | for Adverse Events |
| NMOSD | neuromyelitis optica spectrum disorder |
| NSD | needle safety device |
| OSDI | Ocular Surface Disease Index |
| PD | pharmacodynamic |
| pop-PK | population-pharmacokinetic |
| PK | pharmacokinetic |
| PRO | patient-reported outcome |
| Q4W | every 4 weeks |
| RO | receptor occupancy |
| sIL-6R | soluble interleukin-6 receptor |
| TED | thyroid eye disease |
| TSHR | thyroid stimulating hormone receptor |
| ULN | upper limit of normal |

### REFERENCES

Allen R C, Bradley E A, Fante, R G, et al. A Perspective on the Current Role of Teprotumumab in Treatment of Thyroid Eye Disease. Ophthalmology. 2021; 128(8):1125-8.
Anvari M, Khalilzadeh O, Esteghamati A, et al. Genetic susceptibility to Graves' ophthalmopathy: the role of polymorphisms in proinflammatory cytokine genes. Eye (Lond). 2010; 24(6):1058-63.
Bahn R S. Graves' ophthalmopathy. N Engl J Med. 2010; 362(8):726-38.
Bahn R S. Autoimmunity and Graves' disease. Clin Pharmacol Ther. 2012; 91(4):577-9.
Bartalena L, Krassas G E, Wiersinga W, et al. Efficacy and safety of three different cumulative doses of intravenous methylprednisolone for moderate to severe and active Graves' orbitopathy. J Clin Endocrinol Metab. 2012; 97(12):4454-63.
Bartalena L. Graves' orbitopathy: imperfect treatments for a rare disease. Eur Thyroid J. 2013b; 2(4):259-69.
Bartalena L, Kahaly G J, Baldeschi L, et al. The 2021 European Group on Graves' orbitopathy (EUGOGO) clinical practice guidelines for the medical management of Graves' orbitopathy. Eur J Endocrinol. 2021; 185(4):G43-G67. Published 2021 Aug. 27. doi:10.1530/EJE-21-0479
Bartalena L, Baldeschi L, Boboridis K, et al. The 2016 European Thyroid Association/European Group on Graves' Orbitopathy Guidelines for the Management of Graves' Orbitopathy. Eur Thyroid J. 2016; 5(1):9-26.
Bartalena L, Baldeschi L, Dickinson A, et al. Consensus statement of the European Group on Graves' orbitopathy (EUGOGO) on management of GO. Eur J Endocrinol. 2008; 158(3):273-285.
Bartley G B. The epidemiologic characteristics and clinical course of ophthalmopathy associated with autoimmune thyroid disease in Olmsted County, Minnesota. Trans Am Ophthalmol Soc. 1994; 92:477-588.
Barrio-Barrio J, Sabater A L, Bonet-Farriol E, et al. Graves' Ophthalmopathy: VISA versus EUGOGO Classification, Assessment, and Management. J Ophthalmol. 2015; 2015:249125.
Belinsky I, Creighton F X Jr, Mahoney N, et al. Teprotumumab and Hearing Loss: Case Series and Proposal for Audiologic Monitoring. Ophthalmic Plast Reconstr Surg. 2022; 38(1):73-78.
Chow A, Silkiss R Z. Teprotumumab-associated chronic hearing loss screening and proposed treatments. BMJ Case Rep. 2022; 15(4):e248335. Published 2022 Apr. 13.
Dik W A, Virakul S, van Steensel L. Current perspectives on the role of orbital fibroblasts in the pathogenesis of Graves' ophthalmopathy. Exp Eye Res. 2016; 142:83-91.
Ding A S, Mahoney N R, Campbell A A, Creighton F X. Sensorineural Hearing Loss After Teprotumumab Therapy for Thyroid Eye Disease: A Case Report. Otol Neurotol. 2022; 43(2):e148-e152.
Dolman P J, Cahill K, Czyz C N, et al. Reliability of estimating ductions in thyroid eye disease: an International Thyroid Eye Disease Society multicenter study. Ophthalmology. 2012; 119(2):382-389.
Douglas R S, Kossler A L, Abrams J, et al. Expert Consensus on the Use of Teprotumumab for the Management of Thyroid Eye Disease Using a Modified-Delphi Approach. J Neuroophthalmol. 2022a; 42(3):334-9.
Douglas R S, Dailey R, Subramanian P S, et al. Proptosis and Diplopia Response With Teprotumumab and Placebo vs the Recommended Treatment Regimen With Intravenous Methylprednisolone in Moderate to Severe Thyroid Eye Disease: A Metaanalysis and Matching-Adjusted Indirect Comparison. JAMA Ophthalmol. 2022b; 140(4):328-35.
Draman M S, Ludgate M. Thyroid eye disease—an update. Expert Rev Ophthalmol. 2016:1-12.
Ezra D G, Krell J, Rose G E, et al. Transcriptome-level microarray expression profiling implicates IGF-1 and Wnt signalling dysregulation in the pathogenesis of thyroid-associated orbitopathy. J Clin Pathol. 2012; 65(7):608-13.
European Group on Graves' Orbitopathy (EUGOGO), Wiersinga W M, Perros P, et al. Clinical assessment of patients with Graves' orbitopathy: the European Group on Graves' Orbitopathy recommendations to generalists, specialists and clinical researchers. Eur J Endocrinol. 2006; 155(3):387-389.
Gerding M N, Terwee C B, Dekker F W, et al. Quality of life in patients with Graves' ophthalmopathy is markedly decreased: measurement by the medical outcomes study instrument. Thyroid. 1997; 7(6):885-9.
Gillespie E F, Smith T J, Douglas R S. Thyroid eye disease: towards an evidence base for treatment in the 21st century. Curr Neurol Neurosci Rep. 2012; 12(3):318-24.
Gupta S, Douglas R S. The pathophysiology of thyroid eye disease: implications for immunotherapy. Curr Opin Ophthalmol 2011; 22:385-90.
Highland J, Gordon S, Reddy D, Patel N. Ototoxicity and Teprotumumab. Ann Otol Rhinol Laryngol. 2022; 131(8):910-913.
Hiromatsu Y, Yang D, Bednarczuk T, et al. Cytokine profiles in eye muscle tissue and orbital fat tissue from patients with thyroid-associated ophthalmopathy. J Clin Endocrinol Metab. 2000; 85(3):1194-9.
ICH International Council for Harmonisation. Addendum on estimands and sensitivity analysis in clinical trials to the guideline on statistical principles for clinical trials E9(R1) [resource on the Internet]. November 201]. Available at: https-J/database.ich.org/sites/default/files/E9-R1 Step4_Guideline_2019_1203.pdf
Kahaly G J, Hardt J, Petrak F, et al. Psychosocial factors in subjects with thyroid-associated ophthalmopathy. Thyroid. 2002; 12(3):237-9.
Kahaly G J, Pitz S, Hommel G, et al. Randomized, single blind trial of intravenous versus oral steroid monotherapy in Graves' orbitopathy. J Clin Endocrinol Metab. 2005; 90(9):5234-40.
Khong J J, McNab A A, Ebeling P R, et al. Pathogenesis of thyroid eye disease: review and update on molecular mechanisms. Br J Ophthalmol. 2016; 100(1):142-50.
Kumar S, Schiefer R, Coenen M J, et al. A stimulatory thyrotropin receptor antibody (M22) and thyrotropin increase interleukin-6 expression and secretion in Graves' orbital preadipocyte fibroblasts. Thyroid. 2010; 20(1):59-65
Perez-Moreiras J V, Alvarez-Lopez A, Gomez E C. Treatment of active corticosteroid-resistant graves' orbitopathy. Ophthalmic Plast Reconstr Surg. 2014; 30(2):162-167.
Perez-Moreiras J V, Gomez-Reino J J, Maneiro J R, et al. Efficacy of Tocilizumab in Patients With Moderate-to-Severe Corticosteroid-Resistant Graves Orbitopathy: A Randomized Clinical Trial. Am J Ophthalmol. 2018; 195:181-90.
Ponto K A, Merkesdal S, Hommel G, et al. Public health relevance of Graves' orbitopathy. J Clin Endocrinol Metab. 2013; 98(1):145-52.
Sanchez-Bilbao L, Martinez-Lopez D, Revenga M, et al. Anti-IL-6 Receptor Tocilizumab in Refractory Graves' Orbitopathy: National Multicenter Observational Study of 48 Patients. J Clin Med. 2020; 9(9):2816.
Schiffman R M, Christianson M D, Jacobsen G, et al. Reliability and validity of the ocular surface disease index. Arch Ophthalmol. 2000; 118:615-621.
Sears C M, Azad A D, Amarikwa L, et al. Hearing Dysfunction After Treatment With Teprotumumab for Thyroid Eye Disease. Am J Ophthalmol. 2022; 240:1-13.
Slowik M, Urbaniak-Kujda D, Bohdanowicz-Pawlak A, et al. CD8+CD28-lymphocytes in peripheral blood and serum concentrations of soluble interleukin 6 receptor are increased in patients with Graves' orbitopathy and correlate with disease activity. Endocr Res. 2012; 37(2):89-95.
Weetman A P, Cohen S, Gatter K C, et al. Immunohistochemical analysis of the retrobulbar tissues in Graves' ophthalmopathy. Clin Exp Immunol. 1989; 75(2):222-7.
Wiersinga W M. Quality of life in Graves' ophthalmopathy. Best Pract Res Clin Endocrinol Metab. 2012; 26:359-70.
Wiersinga W M, Bartalena L. Epidemiology and prevention of Graves' ophthalmopathy. Thyroid. 2002; 12(10):855-60.
Terwee C B, Prummel M F, Gerding M N, et al. Measuring disease activity to predict therapeutic outcome in Graves' ophthalmopathy. Clin Endocrinol (Oxf). 2005; 62(2):145-55.
UpToDate, Inc. Treatment of Graves' orbitopathy (ophthalmopathy) [resource available on the internet]. 2020 [updated 14 May 2020; cited 13 Sep. 2022].
Available from: https://www.uptodate.com/contents/treatment-of-graves-orbitopathy-ophthalmopathy Yu C Y, Correa T, Simmons B A, Hansen M R, Shriver E M. Audiology findings in patients with teprotumumab associated otologic symptoms. Am J Ophthalmol Case Rep. 2021; 24:101202. Published 2021 Sep. 16.
Zang S, Ponto K A, Kahaly G J. Clinical review: Intravenous glucocorticoids for Graves' orbitopathy: efficacy and morbidity. J Clin Endocrinol Metab. 2011; 96(2):320-32.
Zhang-Nunes S X, Dang S, Garneau H C, et al. Characterization and outcomes of repeat orbital decompression for thyroid-associated orbitopathy. Orbit. 2015; 34(2):57-65.

1. A method of treating or preventing thyroid eye disease (TED) in a subject, the method comprising administering to the subject an effective amount of satralizumab.

2. The method of claim 1, wherein the subject has moderate-to-severe active TED.

3. The method of claim 1, wherein the subject has chronic inactive TED.

4. The method of claim 1, wherein the subject is a current smoker.

5. The method of claim 1, wherein the subject is a former smoker.

6. The method of claim 1, wherein the subject has never smoked.

7. The method of any one of claims 1-6, wherein satralizumab is administered to the subject subcutaneously.

8. The method of any one of claims 1-6, wherein a 60 mg, 120 mg, 180 mg, or 240 mg dosage unit of satralizumab is administered to the subject.

9. The method of any one of claims 1-6, wherein satralizumab is administered to the subject every two weeks (Q2W) for three times, and thereafter every four weeks (Q4W).

10. The method of any one of claims 1-6, wherein satralizumab is administered to the subject at least seven times.
